(19) United States
(12) Patent Application Publication
(10) Pub. No.: US 2014/0147013 A1
(43) Pub. Date: May 29, 2014

(54) DIRECT ECHO PARTICLE IMAGE VELOCIMETRY FLOW VECTOR MAPPING ON ULTRASOUND DICOM IMAGES

(71) Applicants: Robin Shandas, Boulder, CO (US); Fuxing Zhang, Ann Arbor, MI (US); Jiusheng Chen, Aurora, CO (US); Luciano A. Mazzaro, Boulder, CO (US)

(72) Inventors: Robin Shandas, Boulder, CO (US); Fuxing Zhang, Ann Arbor, MI (US); Jiusheng Chen, Aurora, CO (US); Luciano A. Mazzaro, Boulder, CO (US)

(73) Assignee: The Regents of the University of Colorado, A Body Corporate, Denver, CO (US)

(21) Appl. No.: 13/879,006

(22) PCT Filed: Oct. 11, 2011
(86) PCT No.: PCT/US11/55842

§ 371 Date: Jun. 25, 2013

Related U.S. Application Data
(60) Provisional application No. 61/392,032, filed on Oct. 11, 2010.

(51) Int. Cl. A61B 8/14; G06T 7/00

(52) U.S. Cl. CPC A61B 8/14; G06T 7/0016
USPC 382/107

(57) ABSTRACT

An Echo PIV analysis process, apparatus and algorithm are developed to reduce noise and analyze DICOM images representing a fluid flow of a plurality of particles. A plurality of DICOM images representing sequential image pairs of a plurality of particles is received. The plurality of DICOM sequential image pairs are grouped. The sequential image pairs are correlated to create N cross correlation maps. An average cross-correlation transformation is applied to each cross correlation map to create an image pair vector map for each image pair. A maximizing operation is applied to one or more of the N adjacent image pair vector maps to create a modified image pair vector map for the one or more of the N image pairs. The maps are combined to create a corresponding temporary vector map that are averaged to obtain a mean velocity vector field of the sequential image pairs.

### CROSS REFERENCE TO RELATED APPLICATIONS

This application claims the benefit of priority pursuant to 35 U.S.C. §119(e) of U.S. provisional application No. 61/392,032 filed 10 Oct. 2010 entitled “Direct echo particle image volocimetry flow vector mapping on ultrasound DICOM images.”

### STATEMENT REGARDING FEDERALLY SPONSORED RESEARCH OR DEVELOPMENT

This technology was developed with sponsorship by the National Science Foundation (NSF) Grant No. CTS-0421461 and National Institute of Health (NIH-HLBI) Grant No. R21 HLO79868 and the U.S. federal government has certain rights to this technology.

### TECHNICAL FIELD

### Background

A majority of all cardiovascular diseases and disorders is related to hemodynamic dysfunction. Developments of many cardiovascular problems such as athermoma, intimal hyperplasia, thrombus and hemolysis have been shown to have a close relationship with arterial flow conditions. In particular, fluid shear stress is considered an important mediator in the development of the above problems. For example, in congenital heart disease and subsequent surgical palliations, fluid shear stresses at the endothelial surface play a critical role in progression of diseases such as pulmonary hypertension. The focal distribution of atherosclerotic plaques in regions of vessel curvature, bifurcation, and branching also suggests that fluid dynamics and vessel geometry play a localizing role in the cause of plaque formation. Thus, accurate measurement of fluid shear stress in arteries is essential both as a prognostic aid and for following disease and treatment progression.

Non-invasive methods with good temporal and spatial resolution that can measure multiple velocity vectors (and thereby local shear stress) in real time are useful for hemodynamic diagnostics. Accurate measurement of fluid shear stress at the endothelial border in arteries and veins is desirable in cardiovascular diagnostics both as a prognostic aid and for following disease and treatment progression. Furthermore, mapping time-resolved velocity fields at arterial bifurcations and other complex geometries should allow precise evaluation of the presence and extent of recirculation regions, flow separations, and secondary flows within the cardiovascular system, which may be especially important in following disease progression, examining the status of implanted prosthetics such as stents, vascular grafts, and prosthetic valves, or quantifying the level of flow adaptation after bypass or other type of shunt surgery.

However, currently there is no direct means, with sufficient accuracy, of obtaining such information. A variety of methods have been examined for measurement of blood velocity components in vivo. Magnetic resonance imaging (MRI) velocimetry provides multiple components of velocity with good spatial resolution; however, the method is cumbersome to use since it requires breath-holds of the patient, collection of data over multiple cycles for ensemble averaging, and possesses relatively poor temporal resolution. Ultrasound Doppler measurement of local velocity has also been examined. Although this method provides greater temporal resolution, conventional Doppler has the problem of angulation error. It is dependent on the angle between the ultrasound beam and the local velocity vector, only provides velocity along the ultrasound beam (1-D velocity), and has difficulty in measuring flow near the blood-wall interface.

The frequencies from the ‘top end’ of the AM band to the ‘bottom end’ of the VHF television band are part of the general range referred to as “radio frequencies” or RF. The term “ultrasonic” applied generically refers to that which is transmitted above the frequencies of audible sound, and nominally includes anything over 20,000 Hz. Frequencies generally used for medical diagnostic ultrasound extend in the RF range about.1-20 MHz, having been produced by ultrasonic transducers. A wide variety of medical diagnostic applications use one or both the echo time and the Doppler shift of the reflected emissions to measure the distance to internal organs and structures and the speed of movement of those structures. Ultrasound imaging near the surface of the body has better resolution than that within the body, as resolution decreases with the depth of penetration. The use of longer wavelengths implies lower resolution since the maximum resolution of any imaging process is proportional to the wavelength of the imaging wave. Ultrasonic medical imaging is conventionally produced by applying the output of an electronic oscillator to a thin wafer of piezoelectric material, such as lead zirconate titanate.

The more-recent development of microbubbles to enhance ultrasound backscatter provides a potential ultrasound-based imaging solution for velocimetry of vascular and other opaque flows. This solution is based on the synthesis of two existing technologies: particle image velocimetry (PIV); and brightness-mode (B-mode) contrast ultrasound echo imaging. Particle image velocimetry (PIV) is a non-intrusive, full field optical measuring method for obtaining multi-component velocity vectors. It is a mature flow diagnostic useful in many areas from aerodynamics to biology. However, current PIV methods are limited to the measurement of flows in transparent media due to the requirement for optical transparency.

The synthesis of PIV and B-mode technologies has been termed, as identified in earlier work of the applicants Echo PIV. A very early stage application of PIV was first reported by Crapper et al., 2000; this publication describes use of a medical ultrasound scanner to image kaolin particles in a study of sediment-laden flows of mud in salt water. PIV was applied to B-mode video images, and speeds of up to 6 cm/s were obtained. Others have, since CRAPPER et al., 2000, used 2D ultrasound speckle velocimetry (USV), a combination of classical ultrasonic Doppler velocimetry and 2D elastography methods, for flow imaging. The USV method can provide velocity vectors by analyzing the acoustic speckle pattern of the flow field, which is seeded with a high concentration of scattering particles. However, this method is limited by the requirement for extremely fast acquisition systems, heterogeneous signals caused by polydispersed particles, and high noise induced by high concentration of scattering particles. The inherent necessity of very high scatterer particle concentrations in particular, seriously limits the application of USV in hemodynamics measurement in living creatures.

Early-stage Echo PIV has been implemented on image data obtained using a commercial/conventional clinical ultrasound apparatus to produce velocity vectors for a rotating flow field created within a beaker driven by a stirring device using 0.01 ml Optison® microbubbles introduced into the flow. The maximum achievable frame rate of the conventional system was 500 image frames per second (fps) at reduced imaging window size. Using such frame rates, Kim et al., 2004 improved the measurable maximum velocity from 6 cm/sec, as reported by Crapper et al., 2000 to 50 cm/sec. In the early-stage studies reported by Kim et al., 2004, two phased array transducers were used. The first transducer had a center frequency of 3.5 MHz and average spatial resolution of 2.5 mm in the axial direction and 5 mm in the lateral direction and the second transducer had an axial resolution of 1.2 mm and lateral resolution of 1.7 mm. These resolutions allowed capture of 2D velocity vectors in steady and pulsating flows. These early-stage studies demonstrated that, for both the velocity range and spatial resolution (thus limiting the dynamic range and maximum value of measurable velocities, the ability to capture transient flow phenomena, and the density of the resulting PIV vector field), early-stage Echo PIV was insufficient for full range vascular blood flow imaging.

Ultrasound speckle velocimetry (USV) mentioned above, was originally suggested as a means to obtain multi-component vectors non-invasively. USV is a combination of classical ultrasonic imaging and 2D elastography methods and was proposed some 15 years ago. The USV method measures velocity indirectly by analyzing variations in the acoustic backscatter speckle pattern within the flow field. Several issues, including poor signal-to-noise ratio when using blood cells for backscatter or requirement of excessive contrast particle seeding density when using contrast agents for backscatter, and loss of correlation in regions of high flow shear, have precluded this method from clinical use.

Doppler has only gained acceptance as a marginally quantitative method for assessing vascular hemodynamics. Estimating shear stress using Doppler measurement of peak or mean velocity can significantly underestimate or overestimate shear values. Since vascular anatomy is frequently oriented parallel to the superficial skin surface, the vascular ultrasound Doppler imaging is inherently limited by the less-than-ideal imaging window where the ultrasound beam is directed almost perpendicularly to the flow direction, making flow measurements highly inaccurate. Even if one were able to gather accurate angle corrections using Doppler, it cannot resolve multi-component velocity vectors.

Determining multi-dimensional velocity fields within opaque fluid flows has posed challenges in many areas of fluids research, ranging from the imaging of flows in complex shapes that are difficult to render in transparent media, to the demanding constraints of flow in the aerated surf zone. In the context of blood flow measurement in human body, the added requirements of measurements made in living creatures has traditionally limited the options and capabilities of flow field instrumentation, even further.

The information included in this Background section of the specification, including any references cited herein and any description or discussion thereof, is included for technical reference purposes only and is not to be regarded subject matter by which the scope of the invention is to be bound.

### SUMMARY

Thus, in one form, the invention comprises a method for processing Digital Imaging and Communications in Medicine (DICOM) encoded ultrasound B-mode images representing a fluid flow of a plurality of particles. A plurality of DICOM encoded ultrasound B-mode sequential images representing sequential image pairs of a plurality of particles is received, such as by a computer. The plurality of DICOM sequential image pairs are grouped into a plurality of M groups of images, wherein each M group comprises a plurality of N sequential image pairs. Within each group, the sequential image pairs are correlated to create N cross correlation maps. Within each group, an average cross-correlation transformation is applied to each cross correlation map to create an image pair vector map for each image pair. A maximizing operation is applied to one or more of the N adjacent image pair vector maps to create a modified image pair vector map for the one or more of the N image pairs. For each group, the image pair vector maps and the modified image pair vector maps are combined to create a corresponding temporary vector map. The temporary vector maps are averaged to obtain a mean velocity vector field of the sequential image pairs representing a fluid flow of a plurality of particles.

Thus, in one form, the invention comprises an apparatus for processing Digital Imaging and Communications in Medicine (DICOM) encoded ultrasound B-mode images representing a fluid flow of a plurality of particles. A tangible computer readable storage medium stores DICOM encoded ultrasound B-mode images, said medium storing processor executable instructions comprising:
instructions for receiving a plurality of DICOM encoded ultrasound B-mode sequential images representing sequential image pairs of a plurality of particles;
instructions for grouping the plurality of DICOM sequential image pairs into a plurality of M groups of images, wherein each M group comprises a plurality of N sequential image pairs;
instructions for correlating, within each group, the sequential image pairs to create N cross correlation maps;
instructions for applying, within each group, an average cross-correlation transformation to each cross correlation map to create an image pair vector map for each image pair;
instructions for performing a maximizing operation to at least one or more of the N adjacent image pair vector maps to create a modified image pair vector map for each N image pair;
instructions for combining, for each group, the image pair vector maps and the at least one or more modified image pair vector maps to create a corresponding temporary vector map for each group; and
instructions for averaging the temporary vector maps to obtain a mean velocity vector field of the sequential image pairs representing a fluid flow of a plurality of particles; and

a processor for accessing the DICOM encoded ultrasound B-mode images stored on the tangible computer readable storage medium and for executing the executable instructions stored on the tangible computer readable storage medium to process the accessed DICOM images.

Detailed hemodynamics may be critical for the early diagnosis of many cardiovascular diseases, given their close association with the initiation and progression of thrombus, intimal hyperplasia, plaque rupture, atherosclerosis and left ventricular dysfunction. Echo PIV technique may provide both qualitative and quantitative local flow information such as multi-component velocity vectors, mechanical wall shear stress, recirculation and local vortex patterns in arteries and opaque flows, and may be especially useful for vascular profiling in human carotid arteries. Direct EchoPIV of DICOM images is an integrative method combing the existing technologies of US brightness mode (B-mode) contrast imaging and digital PIV and is validated both in vivo and in vitro through post-processing of radio-frequency (RF) raw backscatter data.

Echo Particle Image Velocimetry (Echo PIV) results generated through the analysis of radio frequency (RF) data have shown to be accurate under in vitro and in vivo test settings for hemodynamic vascular profiling. However, most ultrasound (US) imaging systems do not provide RF data but provide image output using the DICOM standard, which introduces noise during post-processing.

An Echo PIV analysis process and algorithm that allows for the generation of multi-component velocity information through the analysis of DICOM-coded ultrasound B-mode images is disclosed herein. This method is a modified version of the analysis method used to analyze RF ultrasound data, but it improves the vector analysis specifically for US DICOM-coded B-mode images. The specific modifications to the method are: (1) the optimization of the parameter adjustment in system control; (2) noise reduction in the cross-correlation map and (3) the employment of an average correlation method. In order to assess the accuracy of the modified Echo PIV method for the analysis of DICOM images, the DICOM results may be compared against the validated Echo PIV method for analysis of RF ultrasound data.

Vascular and cardiac investigators may find such a tool useful, as will those investigating non-biomedical environments, such as industrial flow, flow of complex polymers, flow near free surfaces and interfaces, and other applications where the opaque nature of the flow field limits the ability to measure multi-component velocity vectors.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention is provided in the following written description of various embodiments of the invention, illustrated in the accompanying drawings, and defined in the appended claims.

### BRIEF DESCRIPTION OF THE DRAWINGS

FIG. 1 is a schematic block diagram of one embodiment of an Echo PIV system according to the invention.

FIGS. 2A, 2B and 2C depict Echo PIV imaging of tube flow according to the invention, with FIG. 2A showing two consecutive grayscale particle images of the microbubbles in the fully developed laminar flow, FIG. 2B showing calculated velocity vectors for the fully developed laminar flow, and FIG. 2C showing a comparison between Echo PIV and the analytic velocity profile.

FIGS. 3A and 3B depict rotating flow measurement obtained using the Echo PIV of the invention, with FIG. 3A showing grayscale particle imaging of particles in the flow and FIG. 3B showing velocity vectors and map.

FIGS. 4A, 4B and 4C depict results from an in vitro Echo PIV study of carotid artery stenosis (artery diameter 8 mm, 70% stenosis): 2D velocity vectors and magnitude map of pre-stenosis (upstream 2-16 mm) in FIG. 4A and post-stenosis section (downstream 16-30 mm) in FIG. 4B, with FIG. 4C showing the flow pattern of the recirculation zone of the post stenosis section.

FIGS. 5A and 5B depict a portion of a B-mode image constructed from digital backscattered RF data of microbubbles within a rotating flow field and the Echo PIV velocity field.

FIG. 6A is a reconstructed B-mode imaging of a jet flow example; the vortex ring at the head of the jet is visible and FIG. 6B depicts the velocity field obtained from Echo PIV analysis of the B-mode image of FIG. 6A.

FIGS. 7A, 7B and 7C illustrates maximums, with FIG. 7A showing the maximum achievable frame rates, FIG. 7B showing the maximum achievable lateral velocities and FIG. 7C showing the maximum achievable velocities with an interrogation window width Ws=3.6 mm and BLD=1.

FIG. 8 is a schematic of the rotating flow setup.

FIG. 9A is one B-mode frame of microbubbles for a transducer focus of 16 mm depth and FIG. 9B is its corresponding Echo PIV velocity vector map.

FIG. 10 illustrates B-mode frame, the corresponding cross-correlation index (CCI) graph, and Echo PIV vectors for bubble concentrations of 6.+−.2.times.10.sup.3/ml (FIGS. 10A, 10B and 10C); 2.+−.0.5.times.10.sup.3/ml (FIGS. 10D, 10E and 10F); 0.2.+−.0.1.times.10.sup.3/ml (FIGS. 10G, 10H and 101).

FIG. 11 illustrates one embodiment of a flow diagram of a main program according to the invention.

FIG. 12 illustrates one embodiment of a flow diagram of a filter selection and parameter setup according to the invention.

FIG. 13 illustrates one embodiment of a flow diagram of a hybrid EPTV/PIV analysis according to the invention.

FIG. 14 illustrates one embodiment of a flow diagram of an adaptive EPIV analysis according to the invention.

FIG. 15 illustrates one embodiment of a flow diagram of a selection of region of interest according to the invention.

FIG. 16 illustrates one embodiment of a flow diagram of an EPIV vector field filtering according to the invention.

FIG. 17 illustrates one embodiment of a flow diagram of a vector field quality report according to the invention.

FIG. 18 illustrates digital B-mode images (left), Echo PTV results (middle) and Echo PIV results (right) for comparison of echo PTV and PIV results in rotating flow with variable bubble concentrations of: 18A shows 150; 18B shows 500; 18C shows 800. Bubble particle images were found within in an area of 20.times.25 mm, according to embodiments of the invention.

FIG. 19 illustrates a schematic of the aneurysm model shown in 19A; B-mode particle image shown in 19B; Echo PIV-measured velocity vectors shown in 19C; and velocity vectors measured using the hybrid Echo PIV/PTV method shown in 19D, according to embodiments of the invention.

FIG. 20 including FIGS. 20A1-20C1 illustrates a comparison of processed and unprocessed B-mode particle images: 20A1 shows original image; 20B1 shows 15.times.15 low pass filtering on 20A1; 20C1 shows 15.times.15 high pass filtering on 20B1, according to embodiments of the invention. FIGS. 20A2-20C2 illustrates velocity vector maps from cross-correlation on processed and unprocessed images: 20A2 shows Original; 20B2 shows after low pass filtering on 20A2; 20C2 shows after high pass filtering on 20B2, according to embodiments of the invention.

FIG. 21 illustrates segments of RF echo signal in B-mode image, according to embodiments of the invention.

FIG. 22 illustrates a comparison of echo signal spectrums: 23A illustrates no filter, 23B illustrates band-pass filtering and 23C illustrates wiener filtering, according to embodiments of the invention.

FIG. 23 illustrates a comparison of echo particle pulses: 23A shows original; 23B shows band-pass filtering; 23C shows wiener filtering.

FIG. 24 illustrates a comparison of particle images: 24A shows original; 24B shows band-pass filtering; 24C shows wiener filtering, according to embodiments of the invention.

FIG. 25 illustrates a comparison of vector maps from cross-correlation based on: 25A shows original images and 25B shows images after wiener filtering, according to embodiments of the invention.

FIG. 26 illustrates a comparison of 26A showing original and 26B showing processed image using wiener filtering method, and the velocity validation results: 26C from unprocessed images, and 26D from processed images.

FIG. 27 illustrates B-mode images and SNR maps before and after correction of intensity non-uniformity of the invention: 27A, 27D shows original; 27B, 27E show after max-min filter; and 27C, 27F shows after high-pass filter.

FIG. 28 illustrates the mean image intensity along columns relative to global mean intensity of the invention: 28A shows original; 28B shows after max-min filtering; 28C shows after max-min filtering followed by high pass filtering.

FIG. 29 illustrates one embodiment of correlation-based template matching (CBTM) for improving RF signal according to the invention.

FIG. 30 illustrates a comparison of echo particle images by using traditional a B-mode construction method and the correlation-based template matching (CBTM) method according to the invention.

FIG. 31 illustrates particle images from rotating flow: 31A with and 31B without the correlation-based template matching (CBTM) method of the invention.

FIG. 32 illustrates velocity field measurement based on echo particle images: 32A with CBTM of the invention and 32B without CBTM.

FIG. 33 illustrates a correlation-SNR map based on echo particle images: 33A with CBTM of the invention and 33B without CBTM.

FIG. 34 illustrates a Standard Gaussian-weighted pulse for use in a matched filter of the invention: 34A shows time history and 34B shows frequency response.

FIG. 35 illustrates a Simulated bubble-scattered pulse for use in a matched filter of the invention: 35A shows time history and 35B shows frequency response.

FIG. 36 illustrates a Measured bubble-scattered pulse for use in a matched filter of the invention: 36A shows time history and 36B shows frequency response.

FIG. 37 illustrates images before and after processing according to one embodiment of the invention: 37A shows no filter; 37B shows template matching by convolution; 37C shows template matching by cross-correlation.

FIG. 38 illustrates vector maps from cross-correlation on B-mode image according to the invention: 38A shows rotating flow; 38B shows the aneurysm model; 38C shows the outlier identified by local filter; and 38D shows the outlier identified by global filter.

FIG. 39 illustrates vector map from rotating flow according to the invention: 39A shows outliers deleted by SNR filter; 39B shows SNR map from cross-correlation; and 39C shows interpolated vector map from 39A.

FIG. 40 illustrates a vector field maps of a rotating flow for different window sizes according to the invention, from an average of 10B-mode images: 40A shows 56.times.56, 40B shows 40.times.40, 40C shows 32.times.32, 40D shows 24.times.24, 40E shows 16.times.16, and 40F shows 8.times.8.

FIG. 41 illustrates a comparison of optical PIV and echo PIV of the invention on vertical central line velocities of a rotating flow field for different interrogation window sizes.

FIG. 42 illustrates the standard derivation of velocity data for averaging under different window sizes according to the invention.

FIG. 43 illustrates a comparison of vector maps by using discrete window offset and conventional cross-correlation with small window sizes, according to the invention: 43A shows 16.times.16, DWO; 43B shows 16.times.16, SCC; 43C shows 8.times.8, DWO; and 43D shows 8.times.8, SCC.

FIG. 44 illustrates a comparison of streamline and velocity contour of an aneurysm model experiment according to the invention: 44A shows dimensions of aneurysm model; 44B shows before; and 44C shows after sub-pixel interpolation.

FIG. 45 illustrates a mesh grid of 3D computational AAA model, according to embodiments of the invention.

FIG. 46 illustrates PIV results for the AAA model, according to embodiments of the invention under steady flow conditions: 46A shows computational fluid dynamics (CFD) simulated velocity vectors and velocity magnitude; 46B shows Echo PIV measured velocity vectors and velocity magnitude; 46C shows streamlines derived from CFD simulation; 46D shows streamlines derived from Echo PIV measurement

FIG. 47 illustrates a comparison of the CFD simulation and Echo PIV results, according to embodiments of the invention: 47A shows CFD grid mesh of AAA model and the placement of line A-A; 47B shows velocity profiles from CFD simulation and Echo PIV measurement.

FIG. 48 illustrates a 3-cycle 5 MHz triangular wave in time and frequency domains, according to embodiments of the invention.

FIG. 49 illustrates a simulated pressure wave form at the focal point, according to embodiments of the invention.

FIG. 50 illustrates a Rayleigh-Plesset (RP) equation simulated bubble backscatter by using the pressure at the focal point for excitation, according to embodiments of the invention

FIG. 51 illustrates a 4 parallel focused beams scanning through a large FOV, according to embodiments of the invention.

FIG. 52 illustrates an estimation of PSF from B-mode microbubble image: 52A shows microbubble images; 52B shows echo pulse from circled bubble in 52A; and 52C shows spectrum of echo pulse in 52B, according to embodiments of the invention.

FIG. 53 illustrates a flow setup for a steady laminar pipe flow in a straight acrylic tube.

FIG. 54A illustrates histograms for velocity vector numbers along a line in an axial direction from 200 single pairs of DICOM-coded images, the vector maps without filtering.

FIG. 54B illustrates histograms for velocity vector numbers along a line in an axial direction from 200 single pairs of RF-reconstructed images, the vector maps after filtering.

FIG. 55 illustrates a method to overcome a low signal-to-noise ratio (SNR) in DICOM B-mode images.

FIG. 56A illustrates histograms of velocity vector distributions computed from 200 single RF image pairs at discrete increments across a diameter of the laminar-flow tube model of FIG. 53.

FIG. 56B illustrates histograms of velocity vector distributions computed from 200 single DICOM image pairs at discrete increments across a diameter of the laminar-flow tube model of FIG. 53 before applying filters.

FIG. 56C illustrates histograms of velocity vector distributions computed from 200 single DICOM image pairs at the same radia locations of FIG. 56B but after filtering.

FIG. 57 illustrates a modified ensemble average method for Echo PIV analysis on DICOM B-mode images.

FIG. 58A illustrates time-averaged velocity vector maps for fully-developed laminar pipe flow from RF data for 100 image pairs.

FIG. 58B illustrates time-averaged velocity vector maps for fully-developed laminar pipe flow from DICOM data, with m=10, n=10.

FIG. 58C illustrates time-averaged velocity vector maps for fully-developed laminar pipe flow from DICOM data, with m=2, n=50.

FIG. 58D illustrates time-averaged velocity vector maps for fully-developed laminar pipe flow from DICOM data, with m=1, n=100.

FIG. 59 illustrates velocity profiles obtained from PIV analysis on 100 pairs of DICOM images with different group numbers and sizes, and compared with theory and RF-based echo PIV results.

FIG. 60A illustrates velocity vector maps for B-mode image of a pulsatile flow model with PIV interrogation area circled by red lines.

FIG. 60B illustrates velocity vector maps for Echo PIV results from RF-data of a pulsatile flow model using original algorithms.

FIG. 60C illustrates velocity vector maps for Echo PIV results from DICOM-data of a pulsatile flow model using original algorithms.

FIG. 60D illustrates velocity vector maps for Echo PIV results from DICOM-data of a pulsatile flow model using new algorithms.

Corresponding reference characters indicate corresponding parts throughout the drawings.

### DETAILED DESCRIPTION

In general, the present invention relates to systems employing methods that couple ultrasound imaging using contrast agents with particle image velocimetry (PIV) methods developed specifically to address issues particular to ultrasound contrast imaging in an effort to characterize the flow of an opaque fluid, such as mammalian blood. More-particularly, the invention is directed to an improved hybrid ultrasound velocimetry method and system that couples PIV and ultrasound contrast imaging, Echo PIV, in a fashion to take advantage of the harmonic radio frequency (RF) backscatter content of contrast agents, such as microbubbles/spheres and other such hollow particles conventionally used—more, of late—as contrast agents (flow tracers) for ultrasound PIV. Components and combinations of features, both hardware and software/processing methods, are disclosed as contemplated herein, such that the system and associated method, not only provide clinicians with additional less-invasive diagnostic and treatment tools, but are also useful in non-clinical imaging applications where velocity fields within opaque structures are sought to be determined non-intrusively. Such applications include but are not limited to pipe flows of fluids, flow of complex fluids such as multi-phase fluids, polymers, etc., flows near free and bounded surfaces, and flows within micro-fabricated devices such as microelectro mechanical systems (MEMS).

Thus, in addition to use within a wide variety of applications related to medical/veterinary diagnostics and treatment of patients—such as for characterization of portions of the cardiovascular system (as further explained, below)—the method is useful for a broader range of industrial opaque flow imaging applications, such as: in the processing of petroleum, the manufacture and processing of beverages (e.g., carbonated liquids including beer and champagne, wine, juice, milk, soybean milk, soft drinks etc.), perfume, ink, water supply, dye, paste, glue, and certain plastics; chemical solution monitoring, for coastal engineering research and analysis; for environmental management of estuaries and coastlines; and so on. Furthermore, by employing high frequency ultrasound, the method can be used for opaque microfluidic imaging measurement, for use in the developing technical area of microfluidic bio-systems.

In one embodiment, the instant invention is directed to an improved method for multi-component blood flow velocimetry for peripheral vascular imaging using Echo PIV. The Echo PIV system developed provides opportunity for increased spatial resolution and dynamic range of measurable velocities. Echo PIV performance is quantified and characterized herein, in a way that optimizes the Echo PIV method to cover a broad range of blood flow, and other, applications. Whereas conventional PIV is centered-around mathematical manipulation of optical images, the combination of system components described and contemplated herein, utilize transducer devices (by way of example, transmit a narrow band ultrasound signal to energize the microbubbles/contrast agent within the flow undergoing examination, and receive the RF emission/backscatter using a broadband receiver transducer, that is to say, the transducer is specifically designed to transmit a narrow band signal and receive a wideband signal) with associated firing sequences and associated PIV analysis, etc. of the harmonic RF ultrasound backscatter content of the contrast agent (e.g., microbubbles by way of example).

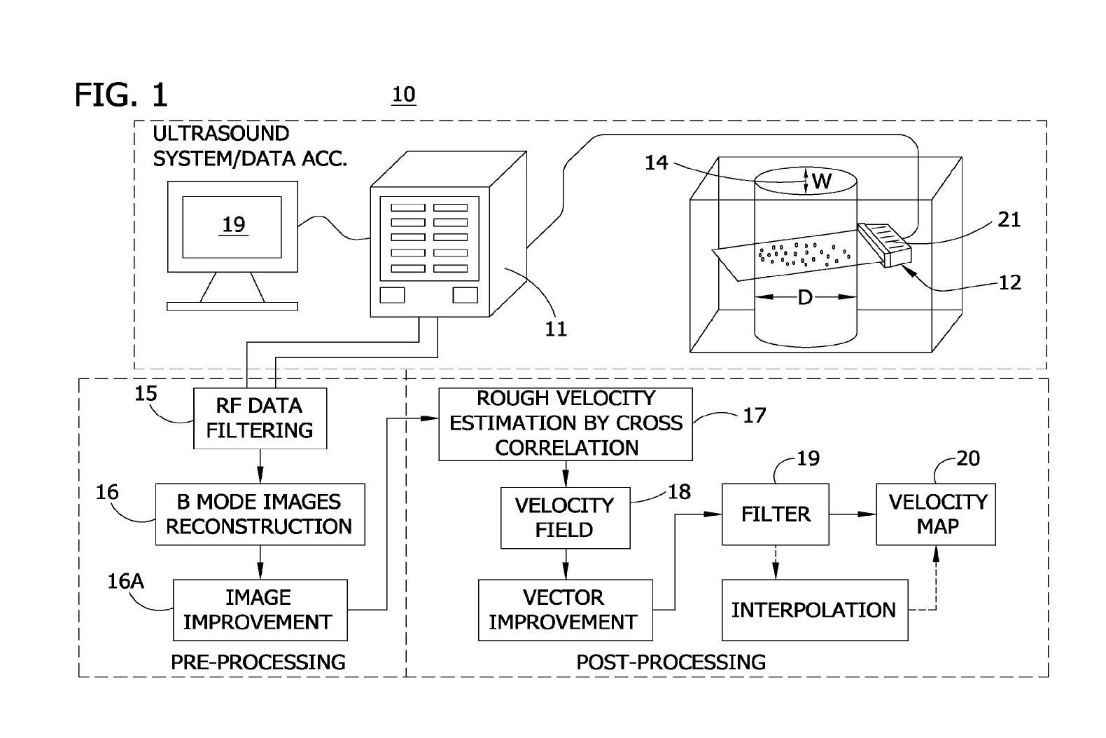

In one embodiment, a system and associated method of the invention generates multi-component blood flow velocimetry for peripheral vascular imaging using Echo PIV. FIG. 1 illustrates overall components of the Echo PIV system 10 and method. B-mode image frames 16 are first obtained by sweeping a focused ultrasound beam, such as produced via ultrasound system 11, linear array transducer 12, and computer 19A through the desired field of view. In one embodiment, the computer 19A comprises a processor and a tangible computer readable storage medium (memory) for storing processor executable instructions for processing images. Transducer 12 are broadband receiver/transmitter devices which transmit a narrow band ultrasound signal to energize the microbubbles/contrast agent within the flow undergoing examination, and receive the RF emission/backscatter as a broadband receiver transducer. The transducer is specifically designed to transmit a narrow band signal and receive a wideband signal with associated firing sequences and associated PIV analysis of the harmonic RF ultrasound backscatter content of the contrast agent (e.g., microbubbles, by way of example).

The ultrasound beam is scattered by contrast microbubbles, which due to their buoyancy characteristics are excellent tracers of the flow field 14. Due to the large difference in impedance between the microbubble and surrounding fluid (collectively referred to by reference character 14), and pressure-induced non-linear resonance, the bubbles scatter strongly. This results in RF DATA 15 which is filtered and represents reconstructed B-mode image frames 16 of the particle positions. Two sequential image frames 16 are improved at 16A then subjected to PIV analysis: the images are divided into interrogation windows (sub-windows); a rough velocity estimation by cross-correlation 17 is performed on the sub-window images to provide the local displacement of the particles; extension of the cross-correlation to all sub-windows over the entire frame allows the velocity vector field 18 to be determined since the time between images (.DELTA.t) is known.

In one embodiment, the method includes identifying and tracking a flow tracer (e.g., ultrasound contrast microbubbles) within a flow field, and computing local velocity vectors using a cross-correlation algorithm. The particle image is obtained by sweeping a focused ultrasonic beam through the desired field of view of the fluid. The processing of RF backscatter data is preferably accomplished using: the RF data integrated to produce the signal intensity at that point in space and time; the RF data is filtered by analyzing and processing to extract only the fundamental harmonic component, which is then used to create the B-mode image so that other harmonic components, including but not limited to the sub-harmonic, the ultra-harmonic, or the second harmonic are eliminated or minimized.

In another embodiment, the method and associated system of obtaining multi-component vectors velocity within opaque flow utilizing ultrasound emissions, includes: identifying and tracking a flow tracer (ultrasound contrast microbubbles or other particulate contrast agent) within an opaque flow field, constructing particle images using digital RF data and computing particle displacements to obtain local velocity vectors over an area under investigation, and using a two dimensional (2D) domain cross-correlation function (such as an FFT) applied to interrogated particle images. This method produces an integrated anatomic and functional examination by providing multi-component velocity data that can be matched to produce an anatomic diagram of the area under investigation. Further, particle tracking can be used instead of particle velocimetry to follow individual traces when used with ultrasound systems imaging at high frequencies.

As noted above, the ultrasound beam is scattered by the ‘contrast agent’ which, by way of example, can include microbubbles of gas seeded into the fluid. Due to the large acoustic impedance mismatch between the bubble and fluid, the bubbles scatter strongly and ‘shine’ acoustically in the ultrasound field, resulting in a clear digital radio-frequency (RF) backscatter of the particle positions with excellent signal to noise ratio (SNR). These RF data are processed to yield the imaging frame for that particular scanning time sequence.

The processing of RF backscatter data is preferably accomplished using certain of the following: the RF data is integrated to produce the signal intensity at that point in space and time; the RF data is analyzed to extract only the fundamental harmonic component, which is then used to create the B-mode image; the RF data is processed to extract any of the harmonic components, including but not limited to the sub-harmonic, the ultra-harmonic, or the second harmonic. The use of contrast microbubbles produces harmonic signatures in the RF signal, which serve to delineate backscatter from bubbles separately from backscatter from tissue. The term Harmonic Echo PIV is used to refer to applications employing the harmonic content of RF.

Two such sequential particle images are, next, subjected to velocimetry analysis: the images are divided into interrogation sub-windows, and the corresponding interrogation windows within each of the two images are then cross-correlated in 2D Fourier space. The cross-correlation between the two images gives the displacement of the particles, allowing a velocity vector field to be determined based on the time between images. The ultrasound velocimetry system is designed to handle high frame rates (up to 2000 frames per second) and can measure real time multi-component flow velocity vectors with large dynamic velocity range up to 2 m/s. The Echo PIV method also provides information on anatomic structures, and thereby allows both structure and functional imaging by showing multi-component velocity data overlain on amplitude echo images of anatomy.

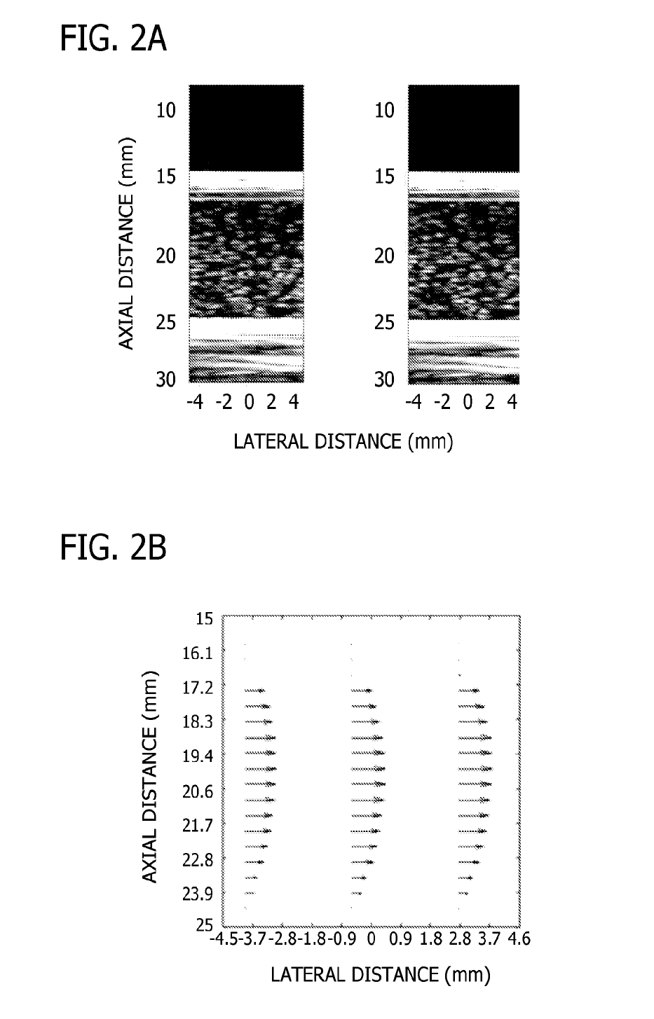

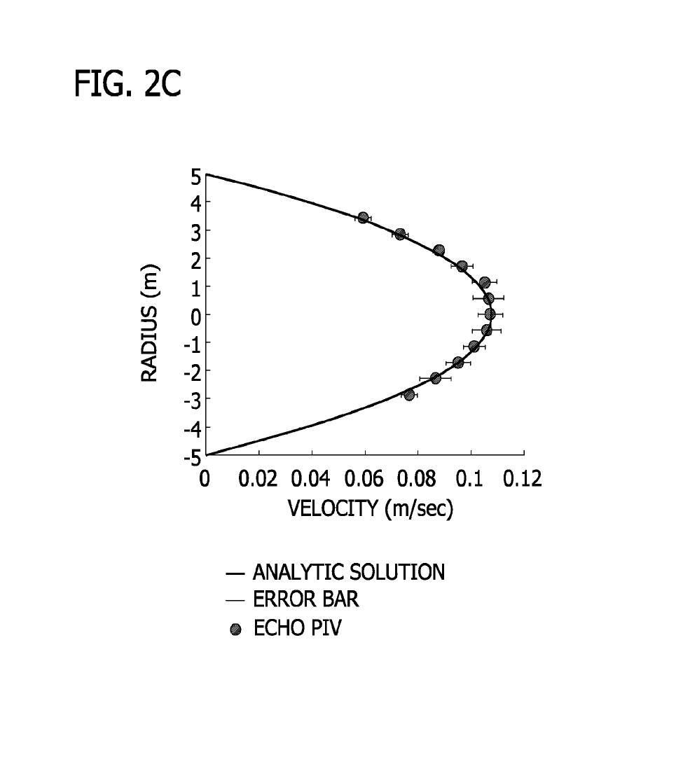

FIGS. 2A, 2B, and 2C illustrate echo PIV imaging of a tube flow. FIG. 2A shows two consecutive grayscale particle images of the microbubbles in the fully developed laminar flow. FIG. 2B shows the calculated velocity vectors for the fully developed laminar flow. FIG. 2C shows a comparison between Echo PIV and the analytic velocity profile.

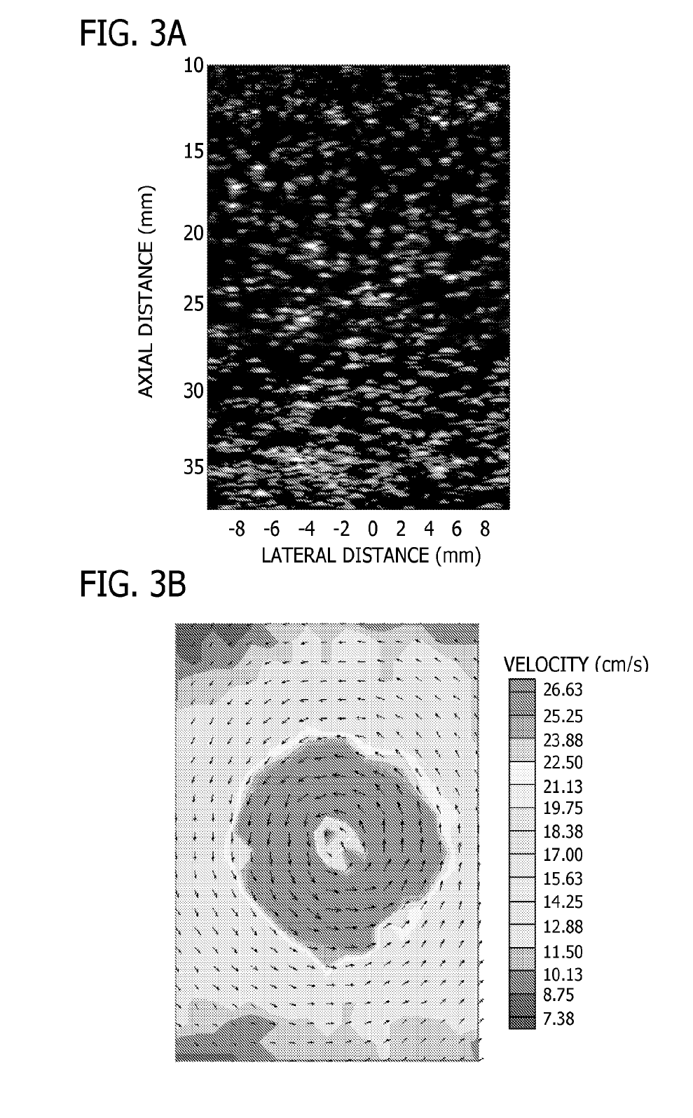

FIGS. 3A and 3B illustrate rotating flow measurement using Echo PIV. FIG. 3A shows grayscale particle imaging of particles in the flow and FIG. 3B shows velocity vectors and a map of their location. The vectors vary from 7.38 cm/s along the perimeter to 26.63 cm/s near the center.

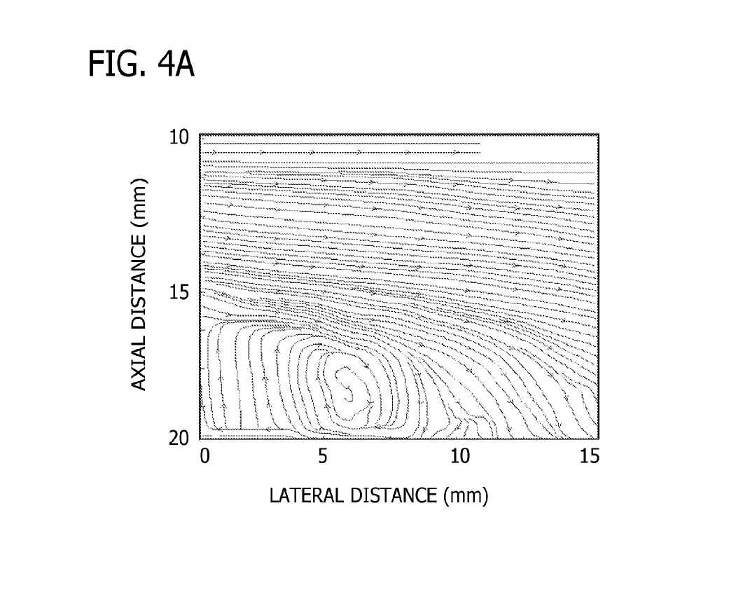

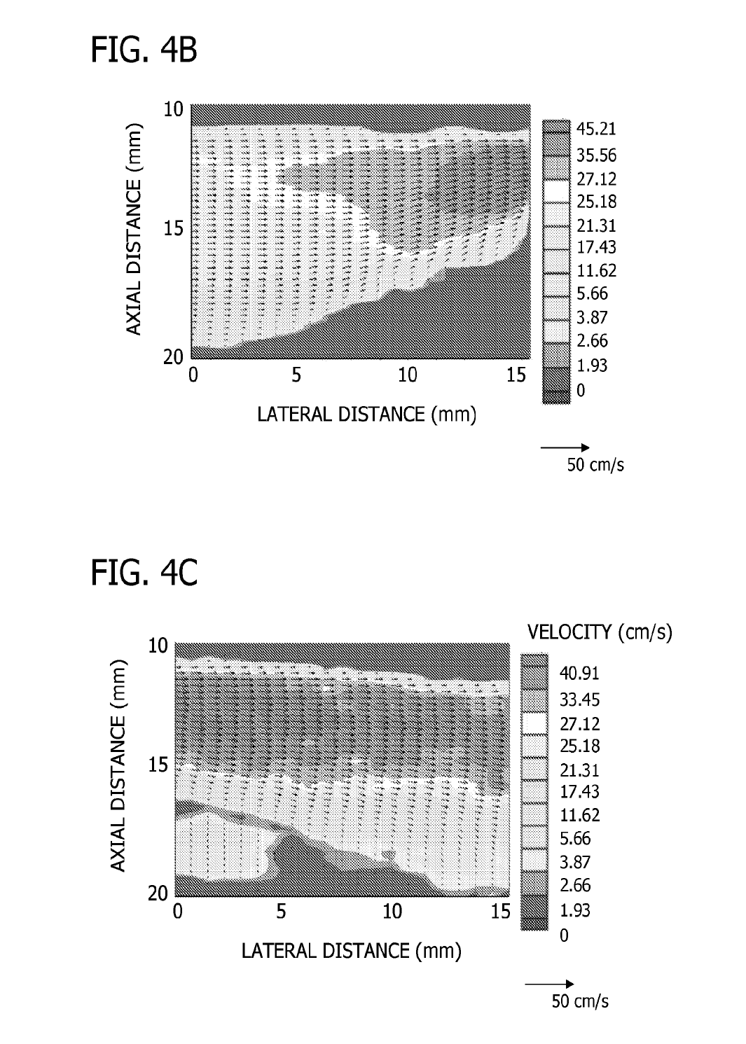

FIGS. 4A, 4B, and 4C illustrate in vitro echo PIV study results of carotid artery stenosis (artery diameter 8 mm, 70% stenosis) showing 2D velocity vectors and magnitude map in FIG. 4A of pre-stenosis (upstream 2-16 mm) and in FIG. 4B of post-stenosis section (downstream 16-30 mm). FIG. 4C shows the flow pattern of the recirculation zone of the post stenosis section.

The cross-correlation system and method of the invention is different from the two dimensional ultrasound velocimetry which uses cross-correlation on received RF signals from directional beam forming previously proposed in U.S. Pat. No. 6,725,076. This previous method applies cross-correlation directly to backscattered RF data (not to the reconstructed images) to obtain velocity vectors. This previous method also does not note or take advantage of acoustically optimized tracer particles such as contrast bubbles. The previous method also does not show the type of firing and receiving protocols needed to control the transducer specifically for optimal particle image velocimetry measurements to be performed. It does not indicate use of harmonic content in the RF backscatter data. It does not use velocimetry algorithms using 2D cross-correlation of interrogated particle images in Fourier Space and velocity data that can be precisely matched with anatomic pictures. It also does not utilize any hybrid velocimetry methods such as combinations of particle tracking and particle image velocimetry. It also does not utilize various pre- and post-processing methods specifically developed to optimize the quality of the velocity vector data. Further, the previous method is not a 2D ultrasound multi-component velocimetry method where ultrasound contrast agents (microbubbles) are used as flow tracers for multi-component velocimetry imaging. Lastly, the previous method is not an ultrasound multi-component velocimetry system which has high frame rates (up to 2000 frame per second) and can measure real time multi-component flow velocity vectors with large dynamic velocity range up to 2 m/s.

Applications to Cardiovascular Blood Flow Imaging

Cardiovascular radiologists, interventionalists, surgeons, and diagnostic imaging experts serving both the adult and pediatric populations can use the invention as a tool: 1) the method and system of the invention provides real time noninvasive measurement of multi-component blood velocity vectors and mapping which is essential both as a prognostic aid and for many cardiovascular disease and treatment progression; 2) the method of the invention is suitable for incorporation into an imaging system having a compact/small footprint to facilitate clinical imaging on and off-site; 3) The method of the invention is adaptable for providing quantitative hemodynamics parameters such as shear stress, vorticity and flow pattern streamlines etc, which are useful in following disease progression to evaluate vulnerable plaques in carotid arteries, anastamotic hyperplasia in vascular grafts, predicting risk of rupture for vascular aneurysms, and so on.

General Opaque Flow Imaging Area

The system and associated method of the invention provides clinicians with additional less-invasive diagnostic and treatment tools, useful in non-clinical imaging applications where velocity fields within opaque structures are sought to be determined non-intrusively. Such applications of the invention include but are not limited to conduit (e.g., pipe) flows of fluids, flow of complex fluids such as multi-phase fluids, polymers, etc., flows near free and bounded surfaces, and flows within micro-fabricated devices such as MEMS.

The following non-limiting examples are provided to further illustrate embodiments of the present invention.

### Example 1

Imaging limits of conventional commercial systems revolve around spatial accuracy and resolution, as well as inherently low frame rates, in turn, limiting the range of measurable velocities, the ability to capture transient flow phenomena, and the density of the resulting PIV vector field. An overall schematic of the Echo PIV system is shown in FIG. 1, and includes host of aspects: automatic-control (via computerized device, such as a PC, for example) of firing sequences; a 7.8 MHz 128-element linear array transducer with element pitch of 0.3 mm and bandwidth of 73%; RF data acquisition; B-mode image generation; and velocimetry algorithms for analyzing the RF-derived B-mode data. The Echo PIV system provides freedom in selecting a much higher range of frame rates (up to 2000 fps) than that of conventional medical ultrasound systems, as well as more freedom in selecting field of view (FOV) (30.about.90 mm), number of transducer elements used to create ultrasound beams (6.about.48 elements), imaging window width (4.about.38 mm), range of multi-focal zones (5.about.75 mm) and power levels (MI: 0.2.about.1.2). Ultrasound contrast microbubbles (Optison® brand contrast agent was used for the illustrated studies) were used as flow tracers and seeded into the flow. Optison® contrast agent has been used clinically for echocardiography: this agent consists of human serum albumin coated microbubbles filled with octafluoropropane and is has a concentration of 5.0.about.8.0.times.108 bubbles/ml; these microbubbles have mean diameters ranging 2.0.about.4.5.mu.m. Other ultrasound contrast agents such as that sold under the Definity® brand may be used.

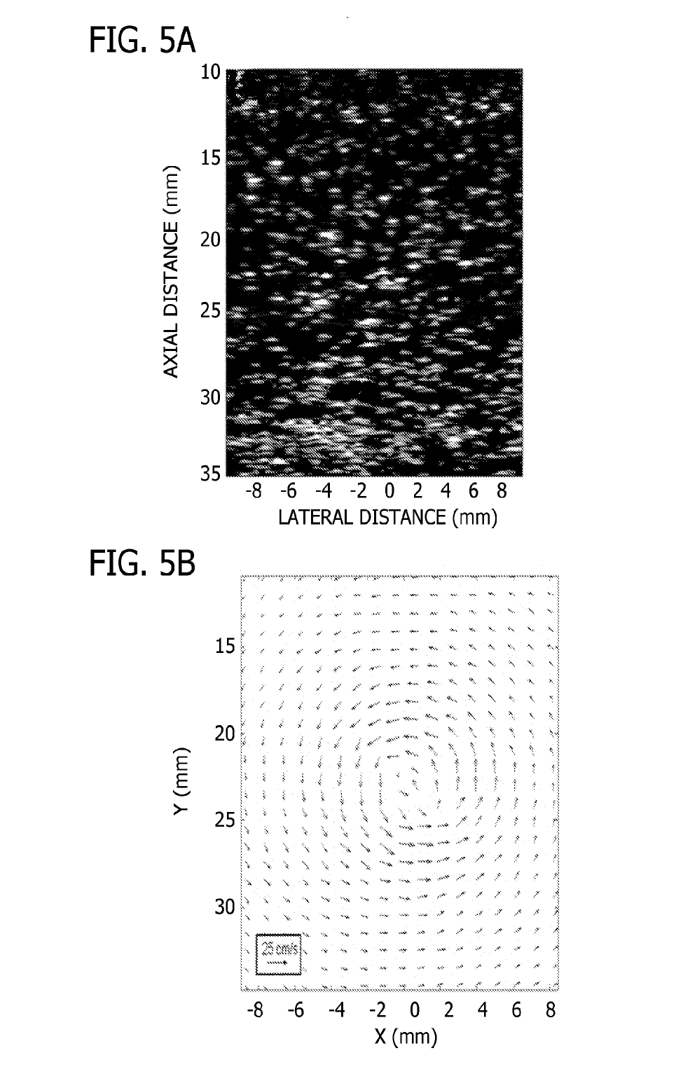

The Echo PIV system and method of the invention uses a much lower concentration of microbubbles than that used by conventional ultrasound contrast imaging: Microbubble concentration for the method can be 12.times.10.sup.3 bubble/ml, roughly 10.sup.5 times less than conventionally used in commercial concentrations. FIG. 5A is a portion of a B-mode image constructed from digital backscattered RF data of microbubbles within a rotating flow field utilizing the Echo PIV system. The flow field for the B-mode image FIG. 5B was generated using a magnet bar placed in a fluid-filled cylindrical plastic tank (42 mm diameter) and driven by a magnetic stirring device. The particle images were obtained at a frame rate of 192 fps using 68 ultrasound beams with a focal depth of 16 mm and FOV of 40 mm. A series of RF datasets corresponding to several frames were acquired. These RF data were reconstructed into image frames and two sequential images were then subjected to PIV analysis. The images were divided into interrogation windows (16.times.16 pixels) and a cross-correlation was applied to the two interrogation windows over the entire imaging frame to determine the displacement of the particles, resulting in the velocity field depicted in FIG. 5B. Other flow configurations, including vortex rings in a jet flow, laminar flow and high velocity pipe flow, were likewise quantified with this method.

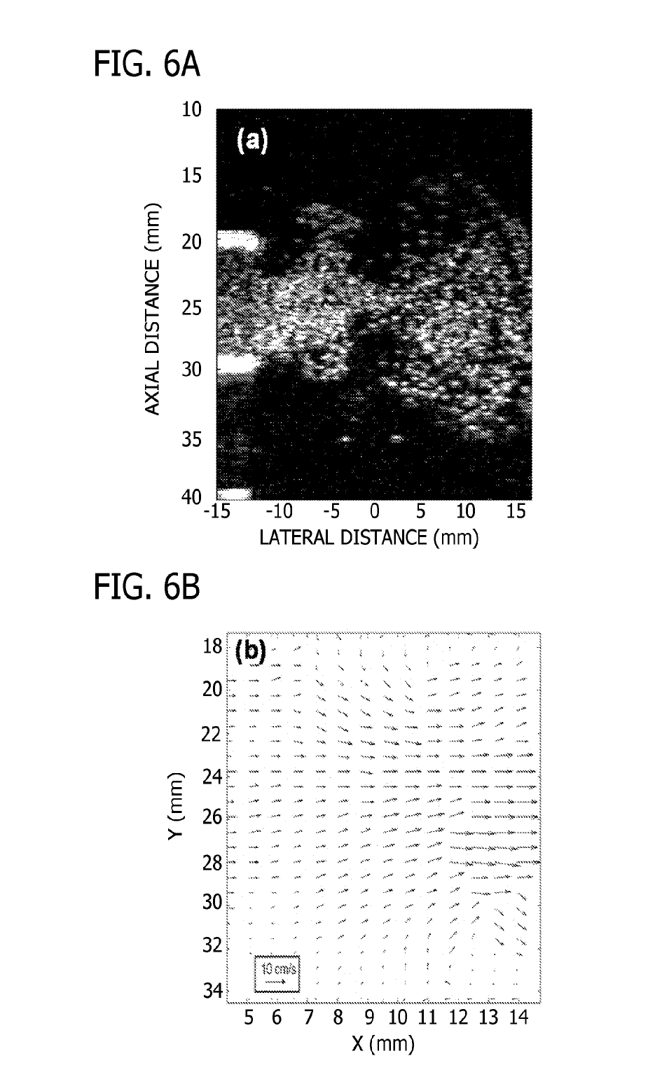

A transient, suddenly started, vortex ring was also imaged using the Echo PIV system and method of the invention. Such a transient flow is difficult to capture using conventional opaque flow velocimetry methods, such as ultrasound Doppler or MRI velocimetry due to the inherently transient nature of the flow and the existence of multi-component velocity vectors. FIG. 6A shows the reconstructed B-mode imaging of the jet flow; the vortex ring at the head of the jet is clearly visible. RF datasets from this flow field were obtained at a frame rate of 90 fps using 104 ultrasound beams with focal depth of 24 mm and FOV of 40 mm. The sub-window size was 20.times.20 pixels. As can be seen from FIG. 6B, Echo PIV provided clear delineation of the velocities in the two vortex rings.

### Example 2

The system of FIG. 1 focuses on two components of Echo PIV of interest: uniformly fine spatial resolution over the entire field of view (FOV) and wide dynamic velocity range. Good spatial resolution prevents bubble images from appearing smeared in the B-mode image 16 (FIG. 1), and maximizes the quality of individual bubble images. This improves the quality of cross-correlation during PIV analysis and increases the accuracy of velocity vector calculation to produce the map 18. However, the exact value of spatial resolution that will be optimal for a particular imaging application will depend on specifications such as the range of velocities to be measured, the diameter of the vessel, and the purpose of the measurement, such as whether shear will be derived. Based on these values, certain transducer operating parameters can be set, such as imaging frequency, pulse length, depth of focus, number of elements used for transmit and receive, order of element firing, etc.

The dynamic velocity range of an Echo PIV system is defined as the difference between the maximum and minimum blood velocities the system can measure. Since blood velocity varies dramatically both around the circulatory system and within a single blood vessel, it is preferable to have ‘wide’ dynamic velocity range for more-optimal performance in different vascular velocimetry applications. Dynamic velocity range is related to both frame rate and spatial resolution. Increasing frame rates allows higher velocities to be measured, while increasing spatial resolution allows lower velocities and higher spatial velocity gradients to be discerned, as is more-fully discussed below, in connection with an example peripheral vascular imaging application.

Specifications for Peripheral Vascular Echo PIV

Peripheral vascular imaging include blood velocity measurements in vessels such as the carotid, brachial, femoral, popliteal, iliac, aortic, and renal arteries, as well as central and peripheral veins. Peripheral vascular imaging using Echo PIV may be accomplished by the system depicted in FIG. 1. The transducer 12 is placed approximately perpendicular to the vessel of interest, and the blood vessel targeted is presumed located at a depth of 5 cm within the tissue sample (e.g., at 14). A rectangular field of view is desired, thus, a linear array transducer 12 is employed. From these general characteristics, certain specific imaging parameters are adopted for the target blood vessel based on: vessel diameter, total FOV, and peak velocities found in the vessel. Vessel diameter dictates the axial resolution required for good Echo PIV data quality. To measure velocity profiles and shear stress, preferably between .about.10-15 velocity vectors will be measured across the vessel lumen (KIM et al., 2004). Thus, for a 1.0 cm vessel, the minimum axial resolution needed would be 1 mm and for a 0.5 cm vessel, the minimum axial resolution needed would be 0.5 mm. In addition, the presence of high shear values within a single interrogation window tends to decrease the quality of cross-correlation, preventing accurate measurements. Transducer firing characteristics—such as pulse length, bandwidth, etc.—are linked with spatial resolution requirements. Good lateral resolution leads to higher quality images; likewise, poor lateral resolution will limit discernment of low velocities. Total FOV is set, based on how much of the vessel geometry is of particular interest. For tortuous or branching vessels, a greater FOV may be desired in order to account for changes in velocity vectors due to variations in local geometry. Use of larger FOVs will impact frame rate, which is also tied to measurable dynamic range of velocities.

Spatial Resolution

Both axial and lateral resolution impact Echo PIV data quality. Axial resolution is heavily dependent on system bandwidth, including that of the transducer. Lateral resolution is determined by the beam width, which in turn is determined by ultrasound frequency, the size of the transducer aperture, the degree of focusing and the imaging depth. As mentioned earlier, the minimum axial resolution preferred is approximately 1/10 of vessel diameter; however, it is advantageous to maximize axial resolution as this will increase Echo PIV vector density within the vessel and increase the accuracy of derivative calculations such as shear stress. For the general vascular Echo PIV imaging characteristics, an axial resolution of .about.200 microns is targeted to provide both good Echo PIV data density and application to a wide range of blood vessel sizes. This level of resolution is obtained by a high frequency (>5 MHz), high bandwidth (>50%) transducer.

Dynamic Velocity Range

The dynamic velocity range, as used herein in connection with the Echo PIV system and method of the invention, is defined as the achievable velocity range between the maximum and minimum resolvable velocity measurement for a fixed set of instrument parameters. As identified in connection with the first-generation Echo PIV system of the applicants, a dynamic velocity range of 1 to 60 cm/sec was reported; this is too low for general vascular imaging use. As identified above, dynamic velocity range is determined by frame rate and spatial resolution of the Echo PIV system. The frame rate is manipulated through flexible control of system parameters, as discussed subsequently. Given the frame rate and spatial resolution, a good estimate for dynamic velocity range is derived employing a velocity calculation algorithm of Echo PIV. Like optical PIV analysis, Echo PIV analysis is based on a cross-correlation method using the FFT algorithm. Certain criteria that apply to optical PIV, has been followed in Echo PIV. Others have carried out Monte-Carlo simulations to determine the requirements for experimental parameters needed to yield optimal optical PIV performance. One recommendation was that in-plane displacement of the particle image should be less than or equal to a quarter of the diameter of the interrogation window (one-quarter rule). The one-quarter rule sets the upper bound of particle displacements in two sequential image frames subject to PIV analysis, and thus determines the maximum velocity the system can measure given a certain frame rate. Since then, other algorithms that move the second window to capture the positions of particles at the later time have evolved. These ‘window offsetting’ methods are limited by the correlation length of the flow itself. The instant Echo PIV system and associated method, do not use such methods.

In one embodiment, the Echo PIV system as set forth in FIG. 1 includes the following features: 128-element 1D linear ultrasound array transducer, control and receiver system, signal processing, Echo PIV processing, and display. The employment of 1D linear ultrasound array transducer provides more uniform axial and lateral resolution over the whole FOV, especially when compared with conventionally used, phased array transducers. This system uses a linear array transducer having a 7.8 MHz center frequency and a 73% fractional bandwidth (6 dB), permitting efficient transmission and receipt of ultrasound pulses in a selected frequency range from .about.5-10 MHz. The width of a single element is 0.3 mm. A computerized processing unit allows flexible control of system parameters, such as the size of imaging widow, focal depth, imaging frequency, beam line density (BLD, detailed later), power level, and so on, permitting ready manipulation of frame rate to achieve quality Echo PIV data. In addition to enabling display of real-time B-mode images, the system enables separate acquisition of the summed RF signal into a high-resolution (16 bit) data acquisition (DAQ) card (such as is commercially available from Gage Applied Technologies, Inc., Canada). B-mode images can be generated selectively for Echo PIV analysis.

FIG. 1 depicts, in block diagram format, signal collecting and processing procedures for the Echo PIV system. In RF data filtering, the linear array transducer scans through the field of view by transmitting and receiving ultrasound pulses sequentially. Backscattered ultrasound is then received by transducer elements and turned into voltage signals which undergo amplification, time gain compensation (TGC) and digitization (analog-to-digital conversion). Then the echo voltages (RF data) pass through digital delay lines for focusing functions and are then summed to produce the resulting scan line. The data acquisition (DAQ) card saves selected summed RF data, which are then used to generate B-mode images for PIV analysis.

Spatial Resolution

Spatial Resolution

Although dynamic focusing has been adopted in ultrasound imaging for purposes of improving image clarity in a large field of view, for vascular blood velocity measurements using Echo PIV, dynamic focusing has not been used. Dynamic focusing decreases the maximum frame rate. Since the diameters of blood vessels are relatively small when compared with imaging depth, and the lateral resolution is quite uniform with depth at the designated focal point where the blood vessel is located, further exploration of dynamic focusing is necessary to determine its usefulness for Echo PIV. An approximate measure of the lateral resolution at the focal point is the Rayleigh resolution criterion which gives the distance from the beam peak to the first zero and is equal to

r=f^{#}λ (Eqn. 1)

where f^{#} is defined as the focal depth divided by the aperture width (OAKLEY, 1991). From equation (1) with f^{#}≈2.5 to 5 and λ=0.2 mm, the lateral resolution of the linear array transducer at different focal depths can be calculated, which ranges approximately from 0.5 mm to 1 mm. The axial resolution Δ is determined generally by the wavelength λ of the incident ultrasound beam and the number N of cycles in the excitation pulse: Δ=λN/2. For our Echo PIV system, with N=2, the axial resolution is about 0.23 mm when the transducer operates at its center frequency of 7.8 MHz.

Temporal Resolution

Frame rate of the Echo PIV system is determined by FOV and several other system parameters, including the beam line density (BLD) and system hardware response time Tₕ BLD is the number of scan lines generated within one transducer element width (Wₑₗₑ) in the B-mode image. The larger the BLD, the larger the number of scan lines composing one image, and the better the lateral spatial resolution. However, there is a tradeoff: higher BLDs require longer times to generate one image and result in decreased frame rates.

In one preferred embodiment, BLD options for the Echo PIV system are 0.5, 1, 2 and 4. The hardware response time Tₕ is the time period between receipt of the most distant echo and transmission of the next beam. For example, Echo PIV system has Tₕ=3 μs. The FOV of the linear array transducer is rectangular-shaped. The width (W_{FOV}) of the FOV is determined by Wₑₗₑ and the number of activated transducer elements (Nₑₗₑ), which ranges from 16 to 128 creating a narrow or wide image. The length of the FOV is determined by the imaging depth (D) required, ranging from 30 mm to 90 mm. Frame rate is directly affected by FOV, since it is inversely proportional to the product of the number of scan lines and the scan time Tₜ for each ultrasound beam, which ranges from 70 μs to 120 μs as the depth increases. The time for generating one imaging frame is:

T_{f}=BLD×T₁×Nₑₗₑ (Eqn. 2)

and the frame rate (FR) is:

$$\begin{matrix}FR = \frac{1}{{T}_{f}} \\ = \frac{1}{BLD \times {T}_{t} \times {N}_{ele}}\end{matrix} \qquad ( Eqn . 3 )$$

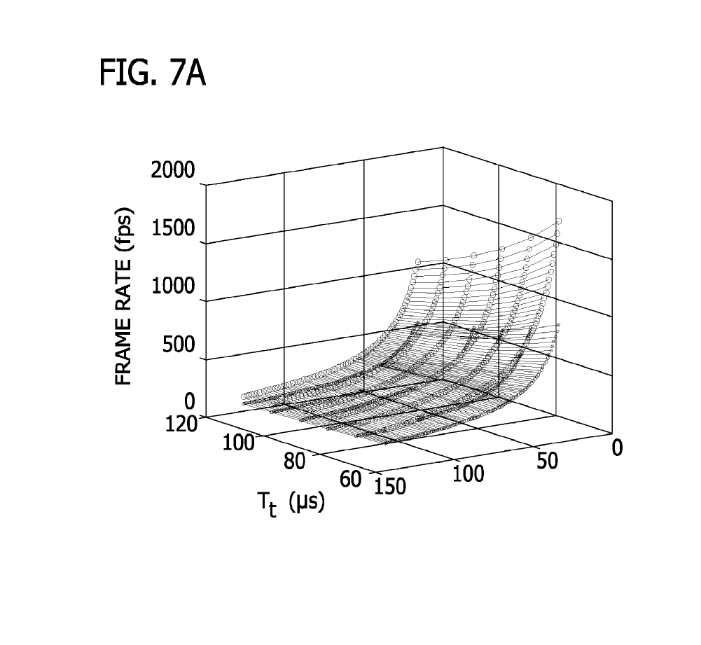

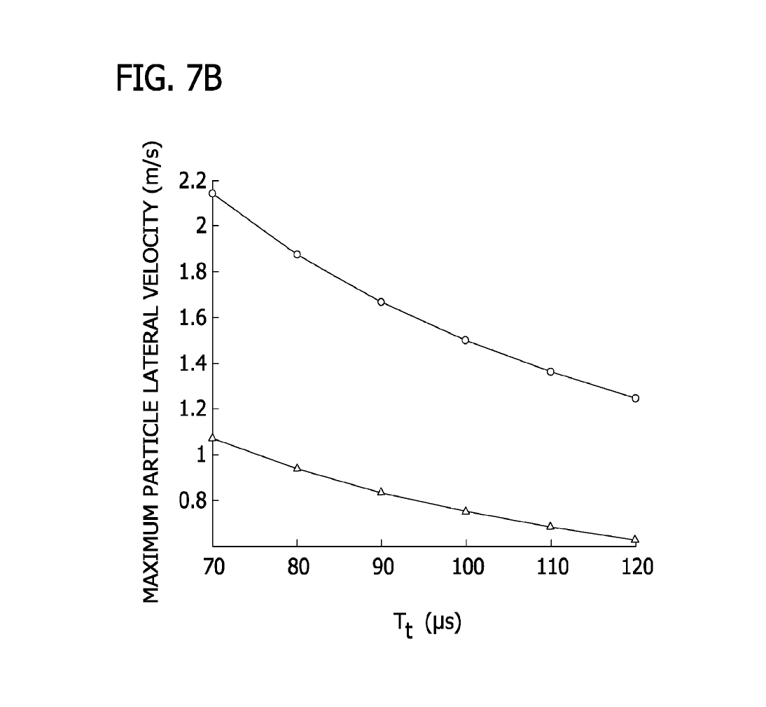

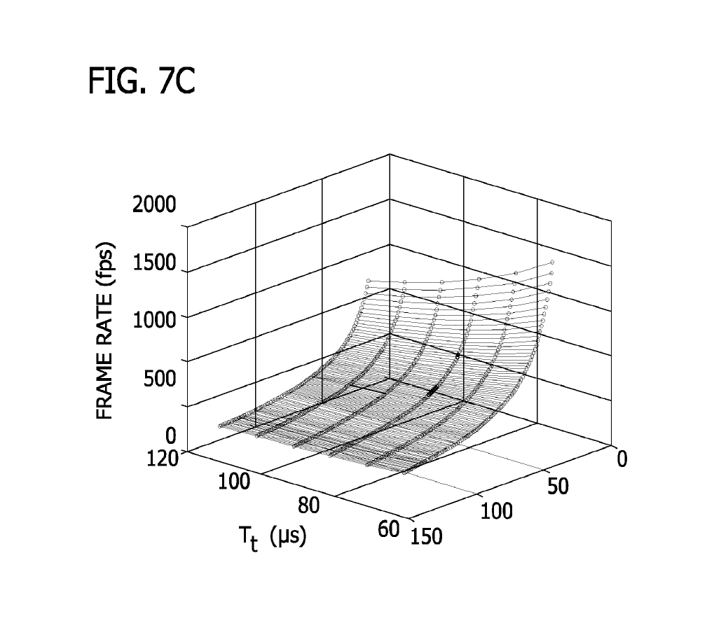

From Eqn. (3), note that frame rate is directly related to the width and length of FOV by Nₑₗₑ and Tₜ, illustrated by FIG. 7A. Thus, for this embodiment of Echo PIV system, if FOV=30 mm by 4.8 mm, the maximum FR is calculated to be 1786 fps when BLD=0.5, and 893 fps when BLD=1.

### Dynamic Velocity Range

In 2D flow field model, a velocity vector may have components in the axial direction and in the lateral direction. For blood velocity measurements, the accuracy of the lateral velocity component is important because the blood vessels will be usually roughly parallel to the transducer scan direction, that is, the primary blood velocity component will usually be in the lateral direction. A derivation of the lateral dynamic velocity range follows below; the same principle applies in the axial direction. The following analysis uses the entire width of the image for a single interrogation window. Applying the one-quarter rule, the maximum lateral displacement of particles in two successive frames is W_{FOV}/4. Given frame rate, maximum lateral velocity that can be measured by for this embodiment of the Echo PIV system is:

$$\begin{matrix}{V}_{tmax} = \frac{{W}_{FOV} / 4}{{T}_{f}} \\ = \frac{FR \times {W}_{FOV}}{4} \\ = \frac{{W}_{ele} \times {N}_{ele}}{4 {T}_{f}} \\ = \frac{{W}_{ele}}{4 \times BLD \times {T}_{t}} \end{matrix} \qquad ( Eqn . 4 )$$

FIG. 7B is a graphical representation of maximum measurable lateral velocities for D=30 mm to 90 mm, using different BLDs, based on an algorithm without window offsetting methods. The highest V_{Imax} is 2.14 m/s when BLD=0.5, and equals 1.07 m/s when BLD=1. Note that this value is independent of the actual width chosen, as long as the distance traveled between images does not exceed the maximum correlation distance for the flow.

Given the frame rate, a conservative estimate for the minimum detectable velocity in the lateral direction V_{Imin} is likewise derived. Thus, a particle image must appear in different beam lines in the second frame for a displacement to be detected: V_{Imin} is actually determined by the spacing of two adjacent beam lines:

$${V}_{lmin} = \frac{{W}_{ele}}{BLD \times {T}_{f}} \qquad ( Eqn . 5 )$$

The minimum detectable velocity in the axial direction is smaller than V_{Imin} since the axial particle displacement is not discretized by beam lines.

The above derivation addresses the case where the whole FOV is employed as an interrogation window for cross-correlation analysis, and only one velocity vector will be generated in the interrogation window, which represents the average particle velocity in this area. It is desirable to know local velocities over the entire field so that a detailed velocity vector map of the flow field can be generated. Echo PIV can generate such maps by dividing the FOV into many sub-windows. The size of the sub-window is determined by the flow characteristics and the level of resolution needed in the vector map. These issues are offset by the requirement for sufficient number of particles within each sub-window. The typical interrogation window sizes we use are 1.8 mm×1.8 mm, 2.7 mm×2.7 mm, 3.6 mm×3.6 mm, 3.6 mm×1.8 mm etc. 5-10 particle images are preferably needed in each interrogation window. Assuming the interrogation window has a width Wₛ, the maximum velocity in the lateral direction that can be measured is:

$$\begin{matrix}{V}_{slmax} = \frac{{W}_{s} / 4}{{T}_{f}} \\ = \frac{{W}_{s}}{4 \times BLD \times {T}_{t} \times {N}_{ele}} \\ = \frac{{W}_{s} \times FR}{4}\end{matrix} \qquad ( Eqn . 6 )$$

Thus, V_{sImin} is reduced from the single window estimate by a factor equal to the number of subwindows used. FIG. 7C plots the maximum measurable velocities with an interrogation window width Wₛ=3.6 mm and BLD=1. Theoretically, a maximum velocity of 0.8 m/s can be measured if the imaging depth D is set to 30 mm (with time per beam Tₜ=70 μm) and 16 transducer elements are activated for imaging (with imaging window width W_{FOV}=4.8 mm).

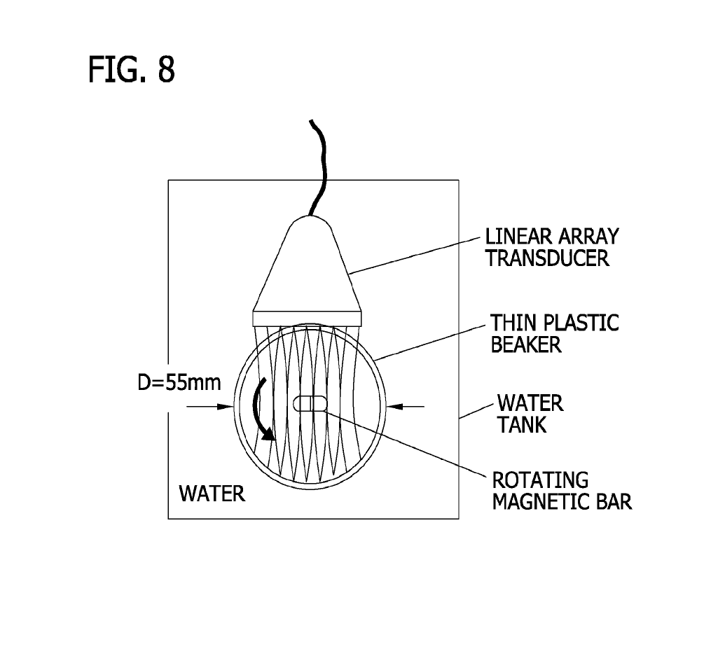

FIG. 8 is a schematic diagram depicting a rotating flow apparatus used for collecting measurements, as follows: The rotating flow was generated in a thin plastic beaker by stirring a magnetic bar with a magnetic stirrer (HI 190M, HANNA Instruments). The 90 mm-high beaker had a diameter of 55 mm and was placed in a rectangular plastic water tank. The linear array transducer was immersed in a water tank for imaging, protected by a waterproof plastic membrane. A 0.012 ml volume of commercially available ultrasound contrast microbubbles, Optison® (Amersham, UK), was injected in the beaker for each measurement. Optison® is a clinically-used contrast agent for echocardiography, and consists of human serum albumin coated microbubbles filled with octafluoropropane with a concentration of 5.0˜8.0×10⁸ bubbles/ml. The microbubbles have mean diameters in the range of 2.0˜4.5 cc m and satisfy the primary requirements for velocimetry: they follow flow trajectories well, and they produce sufficient backscatter for analysis. The microbubbles are stable enough to last for several cardiac cycles, but are eventually destroyed. As explained next, the effects of focal depth and bubble concentration on Echo PIV vector quality were examined.

Focal Depth

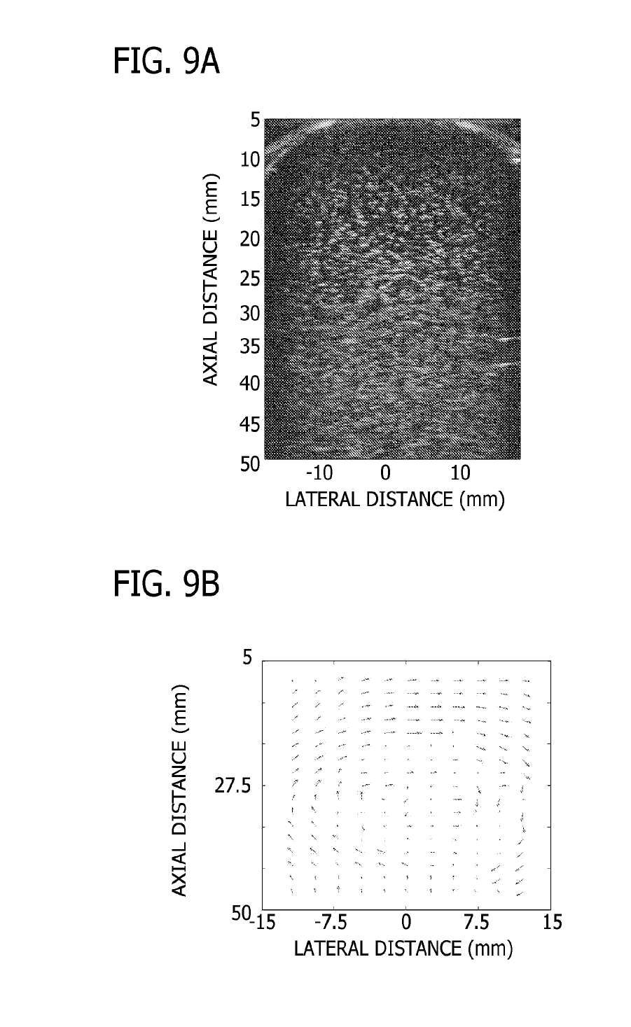

FIG. 9A is one B-mode frame of the microbubbles with a 16 mm focal depth. FIG. 9B shows the resultant Echo PIV velocity vector map using an interrogation window size of 3 mm×3 mm with a 60% overlap. FIGS. 9A and 9B show that focal depth affects Echo PIV results: The bubbles proximal to and within the focal regions are clearly imaged, and velocity vectors of good quality are generated successfully in these regions. On the other hand, bubbles in the far field distal to the focal zone were not resolved with sufficient clarity, resulting in spurious Echo PIV velocity vectors. To generate accurate velocity vectors in a relatively large FOV, the focal depth should be set at the center of or slightly distal to the imaging area, so that the most bubbles can be resolved sufficiently for Echo Ply.

Bubble Concentration

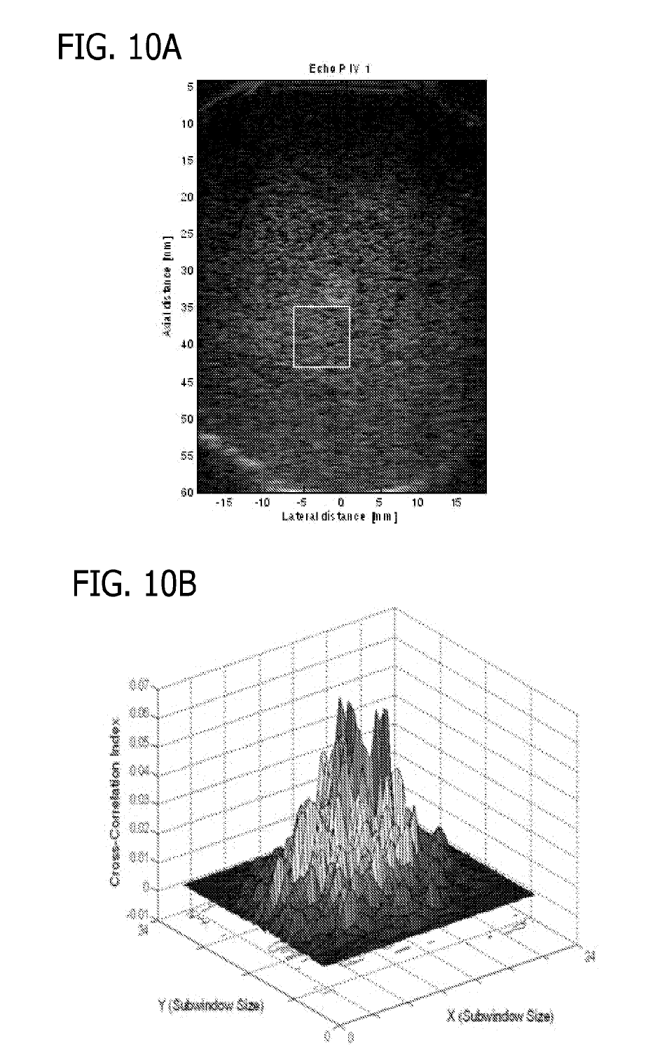

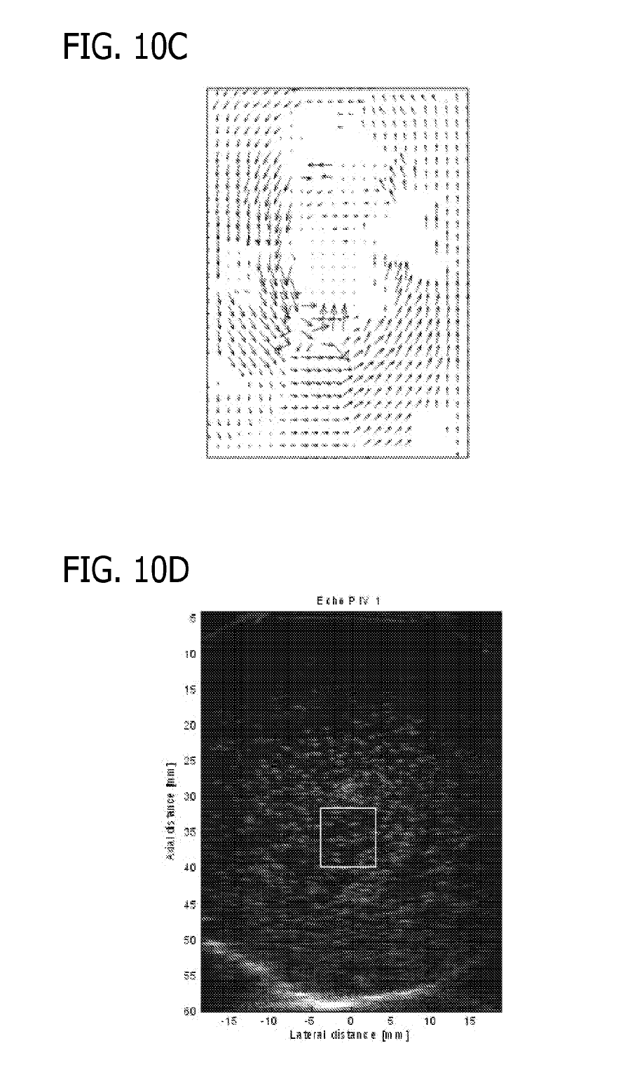

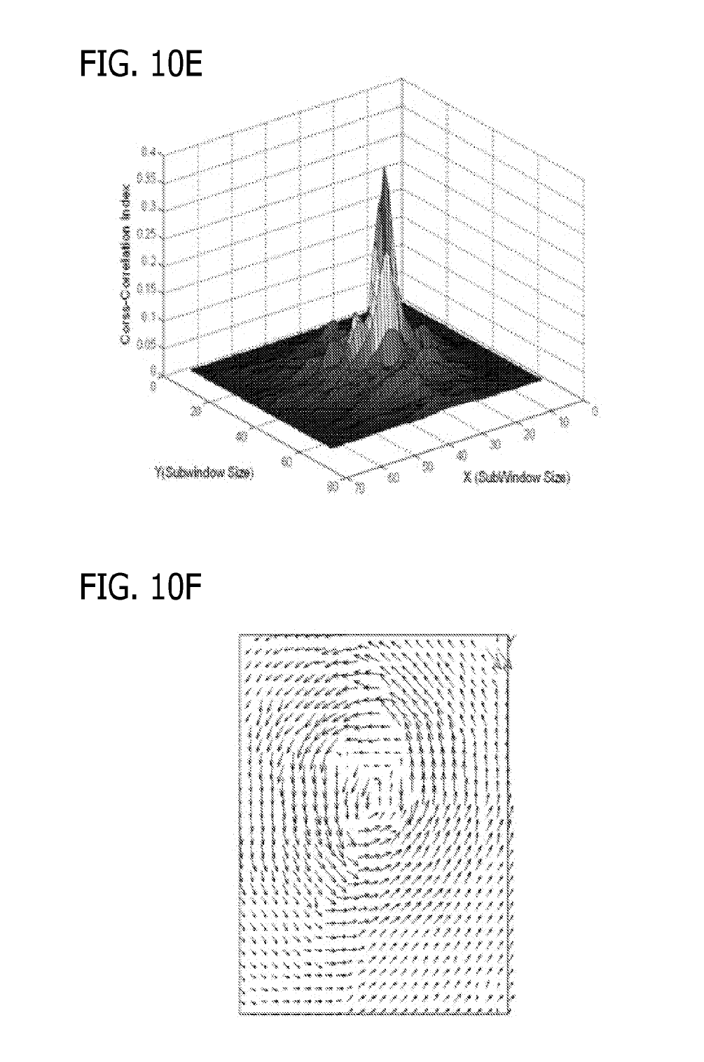

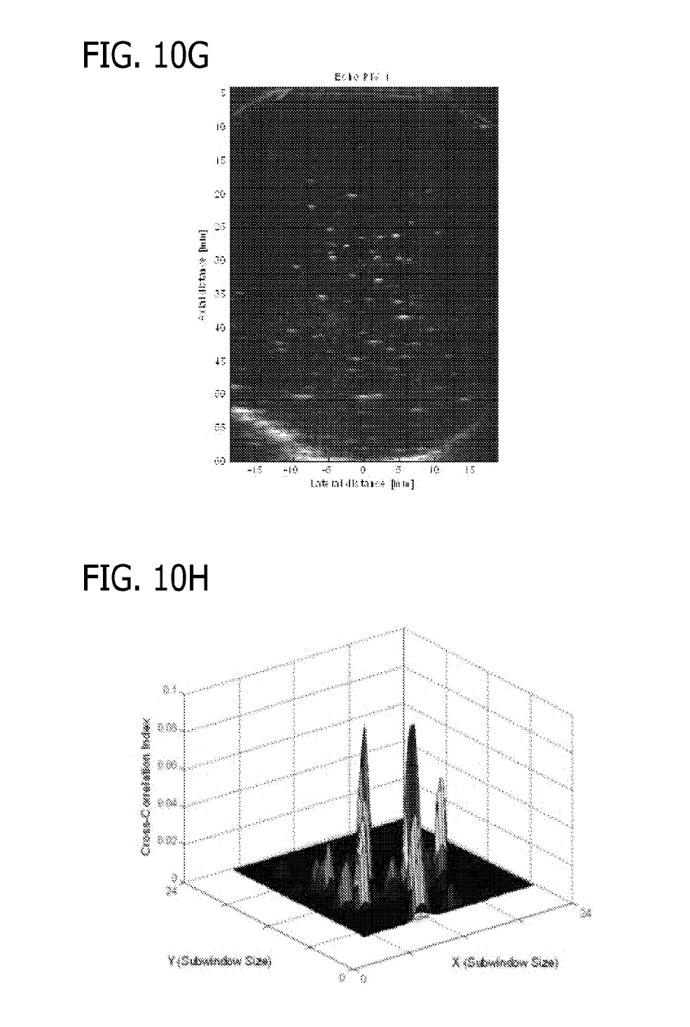

According to one embodiment of the invention, a cross-correlation index (CCI) is used, having been produced by the cross-correlation function (to indicate effectiveness of the pattern-matching between the two sub-windows). Normal CCI values for quality PIV data are within the range of 0.2˜0.8. FIG. 10 show RF constructed B-mode images and the sub-windows analyzed, the corresponding CCI graph and resulting velocity vectors. Initial data in FIGS. 10A, 10B and 100 were obtained at a bubble concentration of (6±2×10³/ml). The CCI graph is flat with peak values around 0.07; the quality of Echo PIV velocity vectors for this image is poor. When bubble concentration was decreased to 2±0.5×10³/ml as shown in FIGS. 10D, 10E and 10F, the CCI graph is more peaked with values around 0.4; the quality of the Echo PIV vectors has improved significantly. Lastly, bubble concentration was further decreased to 0.2±0.1×10³/ml as shown in FIGS. 10G, 10H and 101. The CCI graph again becomes flat, with peak values around 0.1, and the quality of resulting vectors is poor. Results from a series of such studies suggest a “sweet spot” for optimal local bubble concentration of around 1˜2×10³/ml. This concentration is about 100 times lower than suggested clinical upper limits for conventional contrast imaging. The CCI can also be used as a real time indicator of optimal bubble concentration during in vivo imaging; when the CCI plot indicates that an optimum concentration has been reached, Echo PIV data acquisition can begin.

As a result, the system according to the invention, depending on the embodiment, provides one or more of the following:
a. Low signal to noise ratio (SNR) for B-mode images. Although the speckle noise contributes to the noise level in both optical PIV and echo Ply, it appears as a more important reason for echo PIV, especially in tissue imaging, in which the speckle artifacts originate from the interferences of ultrasound backscatter from microbubbles and tissue. Moreover, the other artifacts, particular for Echo PIV, such as the ring-down artifact, section-thickness artifact, grating lobes, mirror range, and range ambiguity, also reduce the quality of B-mode bubble images.
b. Low number of bubble image pairs in each interrogation window. In optical Ply, the number of particle images inside an interrogation window is a stochastic variable with a Poisson probability distribution. Typically an average of 10 particle images per interrogation window can yield a 95% probability to find at least four particle-image pairs. For echo PIV, this does not usually happen due to the limitation of bubble concentration. In fact, the bubble concentration is closely related to the cross-correlation quality between two consecutive B-mode images. The optimal bubble concentration value is typically around 2.+−.0.5×10³/ml, for our employed Optison® (Amersham, UK) microbubbles with diameter range of 2˜5 μm (This concentration is applicable since it is about 100 times lower than suggested clinical upper limits for conventional contrast imaging). For this optimal concentration, generally 4˜6 bubble images are found in each interrogation window with a size from 24×24 to 36×36 pixels. Although the number of bubble images in each interrogation window could be increased by enlarging the window size, this will cause a reduction in the resolution of the velocity vector map and consequently the accuracy of estimated velocity, which is discussed below in further detail.
c. Inherent limitations in spatial resolution caused by the transducer working frequency and other parameters. The 7.5 MHz linear array transducer in the echo PIV system has an axial resolution of about 0.23 mm, which is mainly determined by the operating frequency and driving pulse length. The lateral resolution depends on many factors, including the operating frequency, the size of the transducer aperture, degree of focusing and imaging depth. For peripheral vascular application, the lateral resolution could be optimized to 0.5 mm in our system. By comparing the bubble diameters (generally in the range of 2˜5 μm) and the image resolutions, we know that it is not possible to image individual bubbles. The bubble images seen in the B-mode images are likely a cluster of several microbubbles, and the number of bubbles within the cluster keeps changing due to the fluid flow, making it difficult if not highly improbable that the cluster seen in one B-mode frame is seen exactly the same as that seen in another sequential B-mode image. In subsequent generations of this system, higher operating frequencies (>10 Mhz) transducer arrays will be employed, which can enable better resolution, thereby improving the B-mode images.
d. Non-uniform intensity of B-mode images. The most important reason for the non-uniformity of B-mode images is the non-uniformity in the focused beam lines. In the longitudinal direction, the beam line is well focused at the focal region, but not at the near and far fields. Along the lateral axis, the magnitudes of ultrasound wave appear as a Gaussian curve, with the maximum value at the center point. Since a B-mode image comprises many beam lines, the non-uniform magnitudes in each beam line lead to non-uniformity of B-mode image intensity. Furthermore, non-uniform bubble distribution within the flow due to the effects of eddies and shearing forces within the liquid also contributes to the non-uniform character of B-mode images.

Due to some or all of the factors mentioned above, the coefficients of cross-correlation of B-mode microbubble images may be much lower than those of optical Ply, which may cause erroneous velocity vectors when standard cross-correlation is directly applied. In one embodiment of the invention, the pre-processing and post-processing and improved algorithms provide accuracy improvement in echo PIV method.

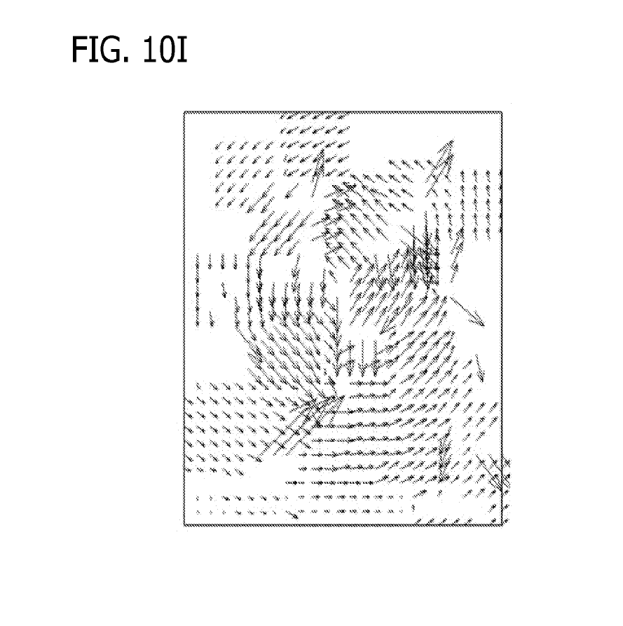

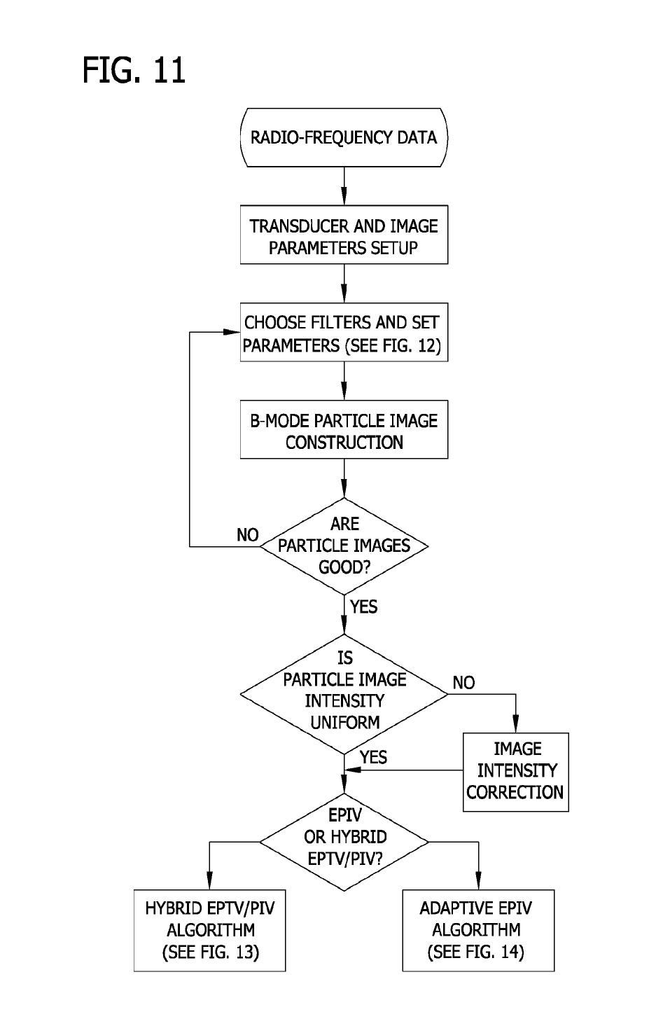

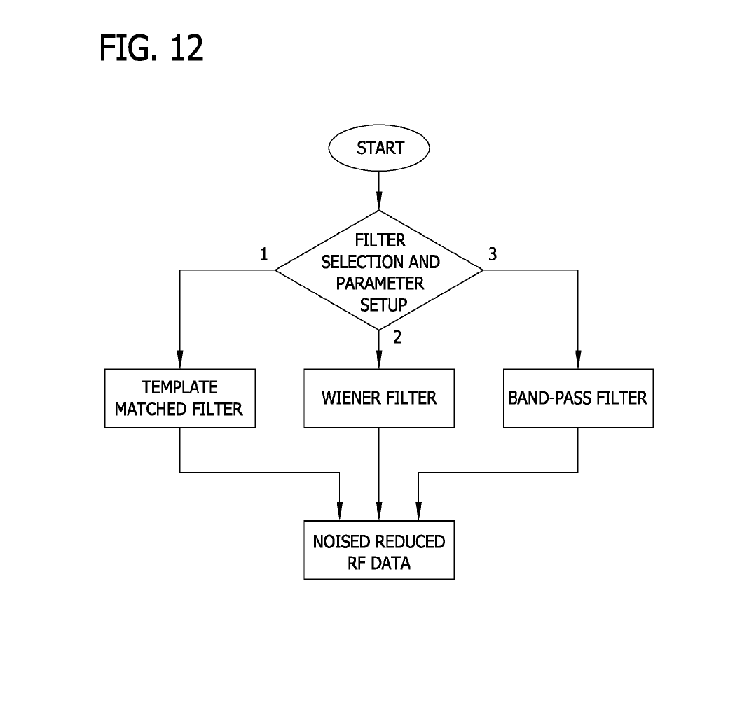

In one embodiment, the ECHO PIV analysis includes the primary analysis (e.g., FIG. 11) and two secondary analysis options (e.g., FIGS. 12, 13 and 14).

The primary analysis of FIG. 11 functions to reconstruct the B-mode particle images from the Radio-Frequency data, and to implement the adaptive EPIV (echo particle image velocity) or hybrid EPTV/EPIV (hybrid echo particle tracking velocimetry/echo particle image velocity analysis). Before the image construction, the RF data is filtered to reduce noise (e.g., FIG. 12). The non-uniform intensity of particle images is corrected, if necessary and the hybrid EPTV/EPIV or adaptive EPIV analysis is carried out based on the selection of user.

As shown in FIG. 12, any one or more of three filters could be used for RF data filtering 15 (FIG. 1) to reduce the noise of RF data. First is the template matched filter. This filter employs the cross correlation between a template signal and the backscattered signal from microbubbles to improve the SNR of RF signals. The parameters to be set include the correlation threshold and the template signals. Second is the wiener filter, where only frequency is required. Third is the bandpass filter, the parameters of which include filter type (IIR or FIR), filter order and frequency band.

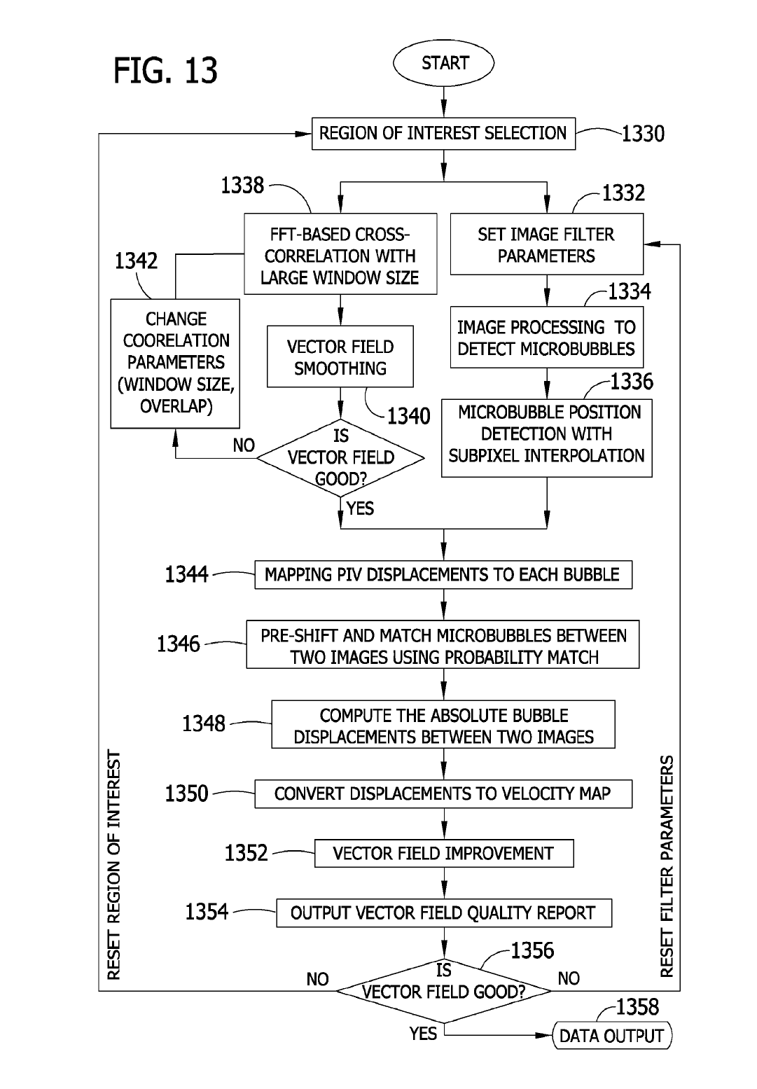

In one embodiment, a hybrid EPTV/EPIV analysis may be employed for post processing as illustrated in FIG. 13:
a. The region of interest (ROI) is first selected at 1330;
b. The parameters are set at 1332 and image processing (e.g., min/max filtering or high pass filtering) is implemented to detect the particles in ROI at 1334 and to detect position at 1336 and create a first image;
c. Simultaneously, in order to estimate the bubble displacements, the cross-correlation between two images is carried out at 1338 with a relative large window size, which keeps the number of outliers at a low level. After smoothing at 1340, if the vector field contains vector dropouts, spurious vectors, and/or is generally of poor quality, the correlation parameters are adjusted at 1342 and correlation performed again until a good vector map (a second image) is obtained;
d. With the vector map from cross-correlation, each particle image velocity (PIV) displacement can be estimated at 1344; this estimate of particle displacements can be used to pre-shift particle positions in the second image, and results compared to the first image at 1346;
e. The accurate particle displacements are calculated at 1348 by using the probability match method, and then the particle tracking velocimetry (PTV) vector map is obtained at 1350;
f. The vector map is improved at 1352 by a vector smoothing algorithm (e.g., min/max filtering or high pass filtering), if necessary;
g. Vector field quality report is output at 1354;
h. If it is determined at 1356 the vector map is not sufficiently high quality in certain region, the user can go back to 1330 to reset the ROI and correlation parameters, or go back to 1332 to reset the filter parameters to reprocess the image; and
i. If a good vector map is obtained at 1356, the data is outputted at 1358 and program is finished.

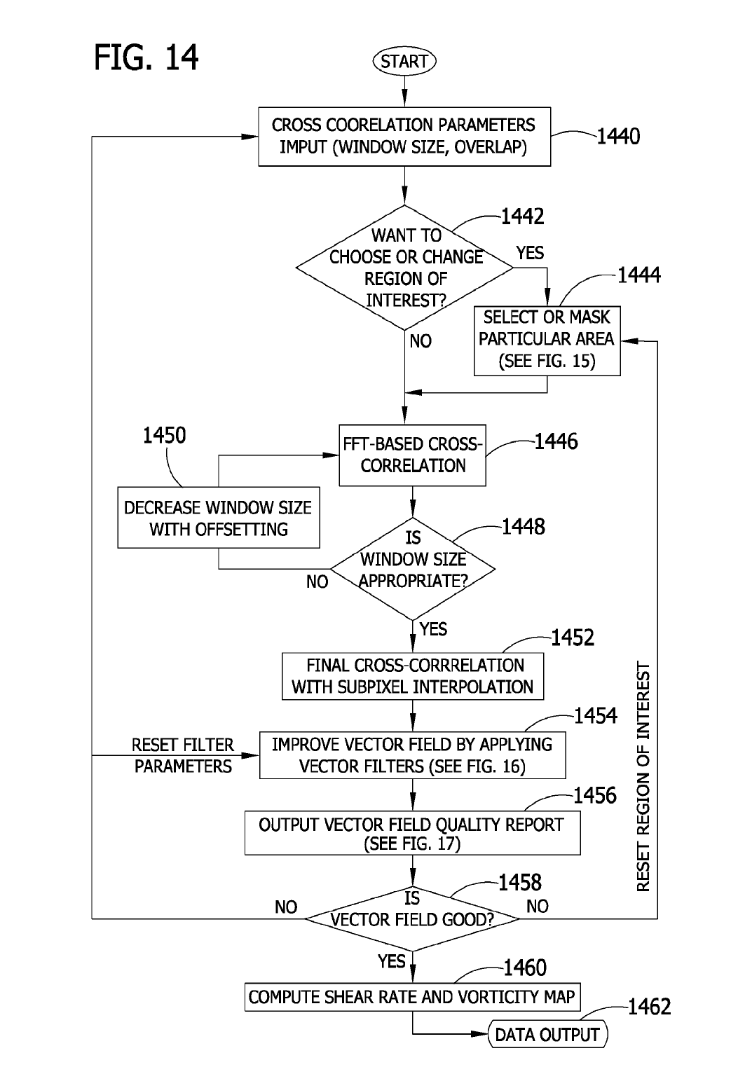

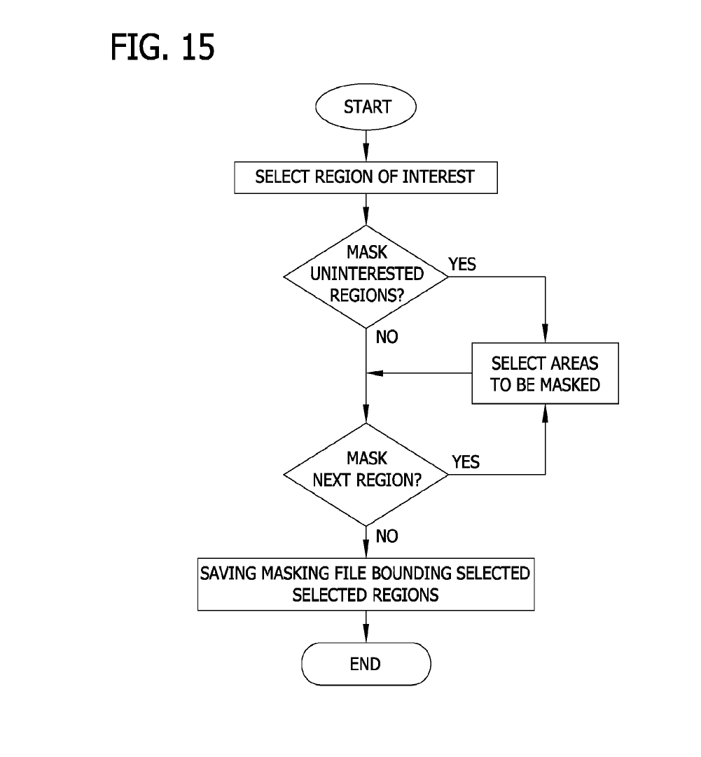

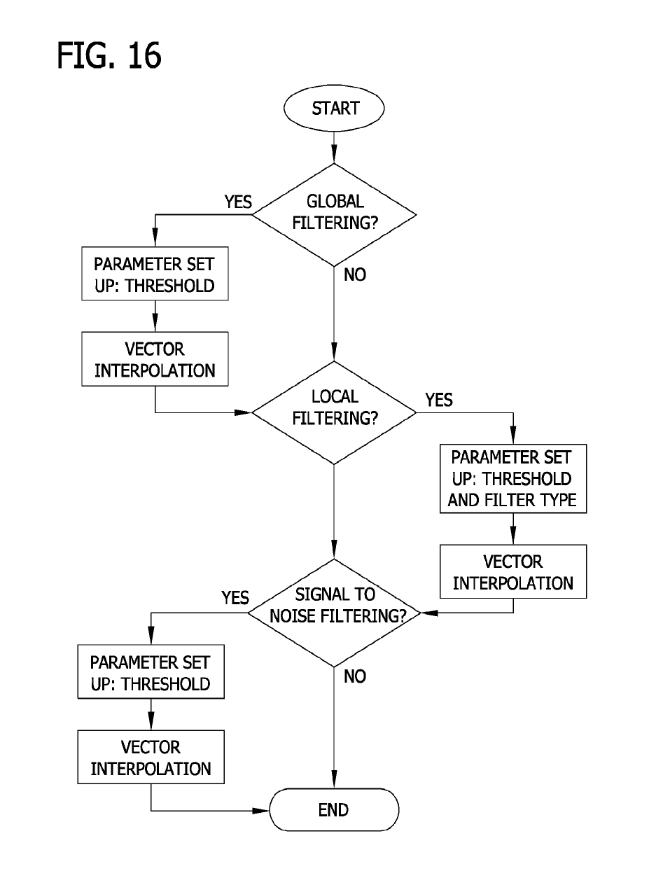

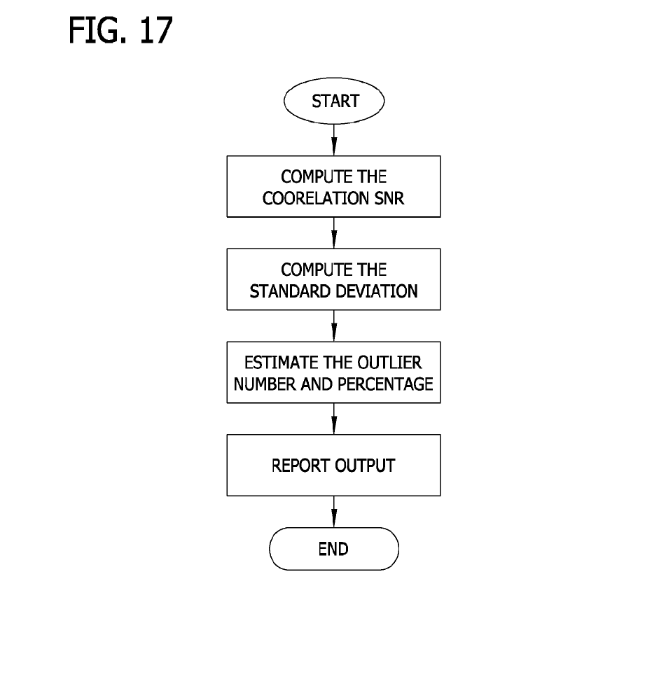

In another embodiment, an adaptive EPIV analysis may be employed as illustrated in FIG. 14:
a. Set the cross-correlation parameters at 1440, including window size, overlap, options for window offset, sub-pixel interpolation;
b. Choose the accurate ROI at 1442, such as by an image masking method at 1444 such as illustrated in FIG. 15 (In order to detect the particles in images, the max-min filter and high pass filter are employed. This can improve the particle image quality and increase the accuracy of probability matching between the two images), if necessary;
c. Fast Fourier transform cross correlation is implemented at 1446;
d. If window size is not appropriate at 1448, reset the window size and overlap, and apply the window offset algorithm at 1450;
e. Apply the final cross-correlation with sub-pixel interpolation to improve the dynamic range of velocity measurement at 1452;
f. Improve the vector field by applying vector filters at 1454, including local filter, global filter and SNR filter as shown in FIG. 16:
   i. Create a vector field with regular grids;
   ii. Map the PTV vectors onto adjacent grids in regular vector field;
   iii. Apply the vector filter on the regular vector field; and
   iv. Map smoothed vectors back to the original PTV grids;
g. Output the vector field quality report at 1456 to evaluate the vector map as shown in FIG. 17;
h. If vector field is not high quality at 1458, three options are available. First, reset the correlation parameters at 1440; Second, reset the ROI at 1444; Third, reset the filter parameters at 1454 and reapply the vector filter;
i. When a good vector map is obtained, the shear rate and vorticity map are computed at 1460; and
j. Output the data at 1462.

In one embodiment, the selecting or marking of an area may be accomplished as illustrated in FIG. 15:
a. Select the region of interest;
b. For a particular field region, such as an aneurysm or stenosis, areas that are needed within ROI can be masked out, if necessary; and
c. Save the boundary file for further use.

In one embodiment, vector filters may be applied as indicated in FIG. 16:
a. Set the global filter threshold, if the global filter is to be used. The vector field is interpolated after filtering;
b. Choose the local filter type (Median or Mean) and set the threshold, if local filter is necessary. Filter is followed by vector interpolation; and
c. Threshold the SNR filter if required, and vector interpolation is applied.

In one embodiment, a vector field quality report may be generated as illustrated in FIG. 17:
a. Compute the correlation SNR (obtained from correlation map);
b. Compute the standard deviation of the vector field;
c. Estimate the outlier number and percentage in vector field; and
d. Output quality report.

In some embodiments, several areas of improvement are seen. First, since Echo PIV is a correlation-based method, it is sensitive to local microbubble concentration; the number of microbubbles within the interrogation window can affect cross-correlation quality and subsequently the accuracy of the derived velocity. Second, conventional PIV has relatively low dynamic range. Although this can be improved using advanced methods such as adaptive window offset and use of sub-pixel or ultra-resolution methods, these algorithms are time-consuming.

Echo PTV was evaluated in vitro using rotating flow and in a model of a vascular aneurysm. Results show that the proposed echo PTV method is less influenced by bubble concentration compared to echo PIV and has a larger dynamic range.

One embodiment of a detailed procedure, from RF data to velocity map, includes: (1) Pre-processing the RF data to minimize noise; (2) reconstructing the B-mode particle image from processed RF data; (3) image improvement and initial velocity estimation via cross-correlation using a larger window size, and vector outlier correction; (4) applying a variety of filters to refine local bubble images; (5) estimating, with sub-pixel resolution, bubble positions in two consecutive images; (6) pre-shifting microbubbles in the second image using previously estimated information, and obtaining the final bubble displacement by matching bubble positions between the two images.

Effect of Bubble Concentration on EPIV and EPTV

Variable bubble concentrations affect both Echo PIV and Echo PTV results. The following relates to a rotating flow model controlled in rotating flow in order to compare the performances of PTV and PIV.

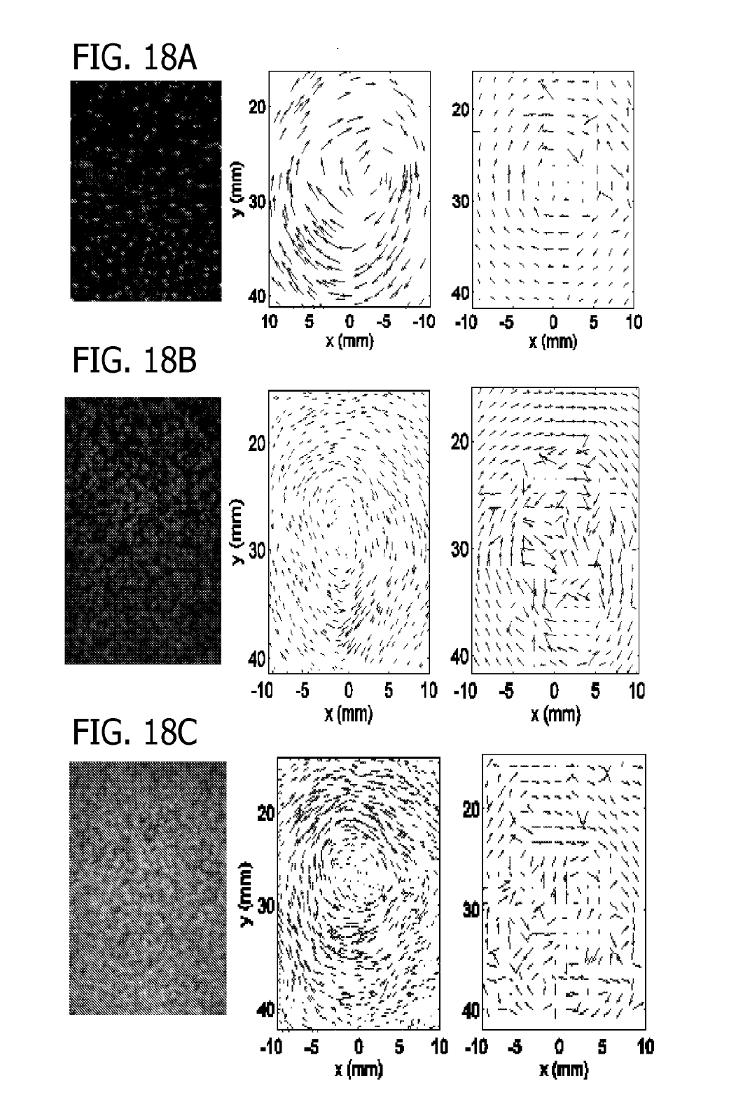

As shown in FIG. 18, from digital B-mode images shown on left, the echo PIV results (right) is much more sensitive to bubble concentration than the Echo PTV results (center). In FIG. 18A, with low bubble concentration, several spurious vectors appear in the center and right corner of the velocity vector map due to the deficiency in matching bubble image pairs. By contrast, very few spurious vectors are found in the lower regions of the Echo PIV map, where bubble concentrations appear adequate for the PIV analysis. As seen in FIG. 18A, the corresponding Echo PTV map shows far fewer spurious vectors, indicating better performance even in the presence of sub-standard microbubble concentrations. As bubble concentration is increased, Echo PIV continues to produce spurious velocity vectors (FIGS. 18B AND 18C), while Echo PTV results maintain consistent quality. Table 1 shows that Echo PTV is capable of producing many more robust velocity vectors within the same flow field than Echo PIV

TABLE 1
Numbers of vectors from echo PIV and PTV maps

| | FIG. 18A | FIG. 18B | FIG. 18C |
|---|---|---|---|
| Echo PTV | 154 | 300 | 221 |
| Echo PTV | 158 | 553 | 864 |

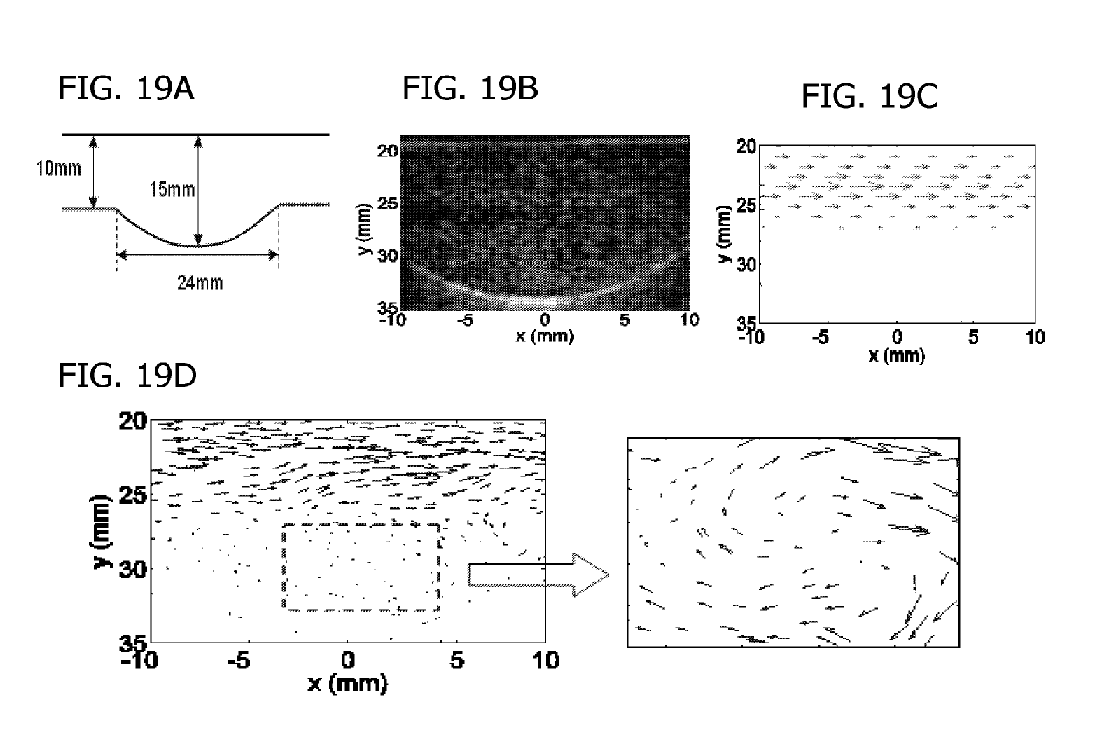

Conventional particle tracking methods possess smaller dynamic range in velocity measurement. This may be overcome by combining PIV and PTV approaches of the invention to create a hybrid Echo PIV/PTV method that maintains the robustness in velocity vector measurement seen in Echo PTV with the larger dynamic range found in Echo Ply. This hybrid method was used to measure velocity vectors within a vascular aneurysm model, which contained a wide range of velocity magnitudes (0.1-10 cm/sec) within the flow field. FIG. 19A illustrates a schematic of the aneurysm model. FIG. 19B shows the B-mode particle image results from the Echo PIV method only; the larger velocities are clearly discerned but the smaller velocities within the aneurysm are not obtained. By contrast, the hybrid Echo PIV/PTV method shown in FIG. 19D (velocity vectors on left and an enlarged, demarcated section on the right) allows clear measurement of the smaller vectors within the aneurysm and the larger velocity vectors in the main flow field.

RF Data Filtering Methods

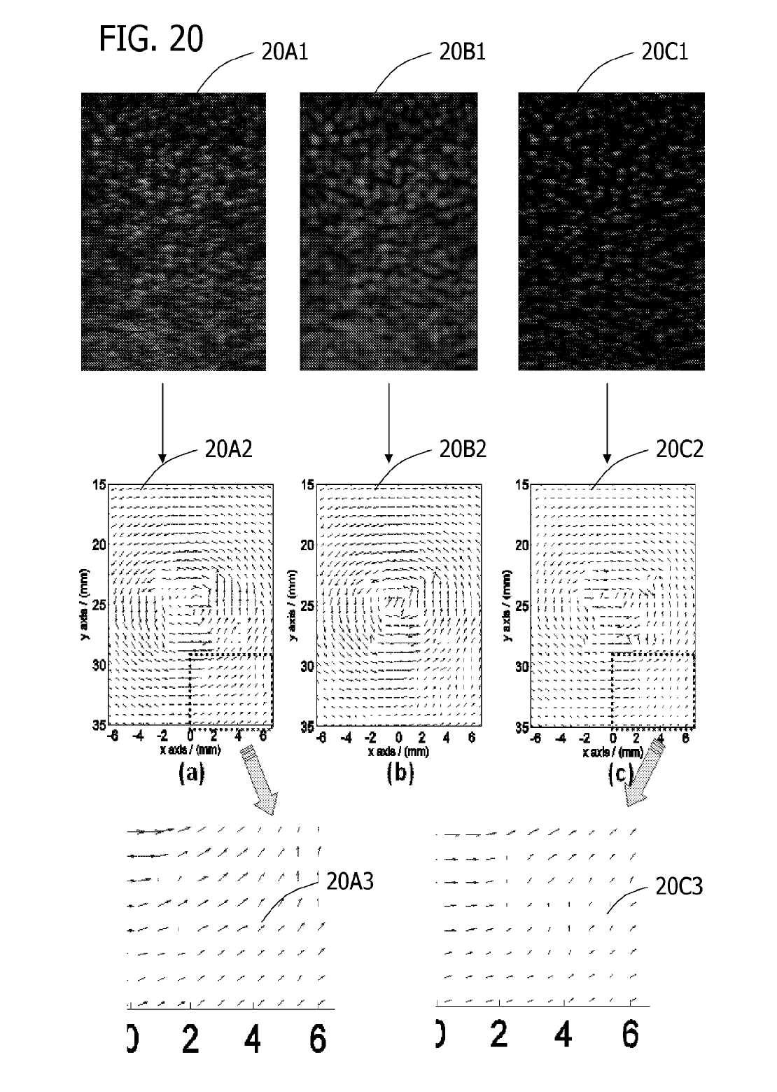

Referring to FIG. 20, FIG. 20A1 shows one embodiment of a one frame of a series of B-mode microbubble images from the rotating flow experiment, which was obtained at a frame rate of 192 fps using 68 ultrasound beams with a focal depth of 24 mm and a field of view (FOV) of 50 mm. By examining this image, we find that: first, both the sizes and shapes of bubble images are not uniform, with some round, some oval and some quite irregular; second, the bubbles images are unrecognizably clustered together at the lower region of the B-mode image due to the high noise level, the low power of the ultrasound waves and low spatial resolutions of transducers at far focal zone. The application of cross-correlation on such images generally produces a velocity vector map with many wrong vectors. One goal of our pre-processing according to one embodiment of the invention is to improve the particle images by reducing or eliminating the noise effects, thereby to improve the vector maps of cross-correlation.

Some conventional image filters can be employed to enhance the quality of particle images, such as low pass filter (median, moving average or wiener filters) to reduce the noise by smoothing the images, and high pass filter to enhance the particle edge. However, we found that such conventional image filters can not work effectively in the processing of echo PIV particle images due to the high noise levels inherent in ultrasound-based imaging.

FIG. 20 including FIGS. 20A1-20C1 illustrates a comparison of processed and unprocessed B-mode particle images: 20A1 is the original image; 20B1 shows 15×15 low pass filtering on 20A1); 20C1 shows 15×15 high pass filtering on 20B1, according to embodiments of the invention. FIGS. 20A2-20C2 illustrates velocity vector maps from cross-correlation on processed and unprocessed images: 20A2 is the original image; 20B2 after low pass filtering on 20A2; 20C2 after high pass filtering on (20B2, according to embodiments of the invention. Based on the microbubble image size, a 15th order (15×15 pixels) low pass filter is employed to reduce the noise. By visual examination, the image appears smoothed. However, the noise level is still high. Increasing filter order causes the image to become more blurred. In the next step, a 15×15 high pass filter is used to enhance the blurred image in FIG. 20B1 and find the particle edges. The background noise in FIG. 20C1 is greatly reduced and the particles are clearly shown.

It is noted that our purpose of image processing is to improve the velocity vector map, not the image itself. Many image processing methods have been utilized for ultrasound imaging to improve ultrasound image quality; however, these do not necessarily increase the quality of the velocity vectors using Echo PIV. By applying the cross-correlation algorithm, we found, in fact, that the vector map is not improved either by the low pass filter or high pass filter. FIGS. 20A2, 20B2 and 20C2 show the velocity vector maps from FIGS. 20A1, 20B1 and 20C1, respectively. FIGS. 20A3 and 20C3 are exploded portions of FIGS. 20A2 and 20C2, respectively. Contrarily, the vector map is even worse in some regions after high-pass filtering. This is because the edge enhancement by high-pass filter produced some incorrect information in the high-noise-level part in FIG. 20B1. In another words, although the signal to noise level is high in FIG. 20C1, some noise information is wrongly recognized as particle information, especially in the lower-right part of the image.

Since conventional image processing methods did not perform well on echo particle images, it is necessary to find better methods to improve the B-mode particle images for Echo PIV processing. As is known, the images for conventional velocimetry methods such as digital PIV (DPIV) or OPIV come directly from digital video cameras; however, the images for echo PIV are reconstructed from ultrasound RF data, in which significant amount of additional information is included. This characteristic provides more flexible methods to improve velocity vector quality through optimized image processing.

The commonly used filter in B-mode RF signals processing is the band-pass filter. Since the spectrum of echo pulse from microbubbles contains a range of frequencies around the exciting signal frequency for the transducer, the noise outside this band could be eliminated by applying a band-pass filter. However, the drawback of this type of filter is that the noise in the band is unaffected. Therefore, we present a wiener filter for pre-processing of our echo PIV images (see Appendix A for design details). To appreciate the performance of this filter, we compared the performances of the wiener filter and the band-pass filter. Again, it must be understood that the particular characteristics of this wiener filter are to optimize Echo PIV velocity vectors, not the ultrasound image per se.

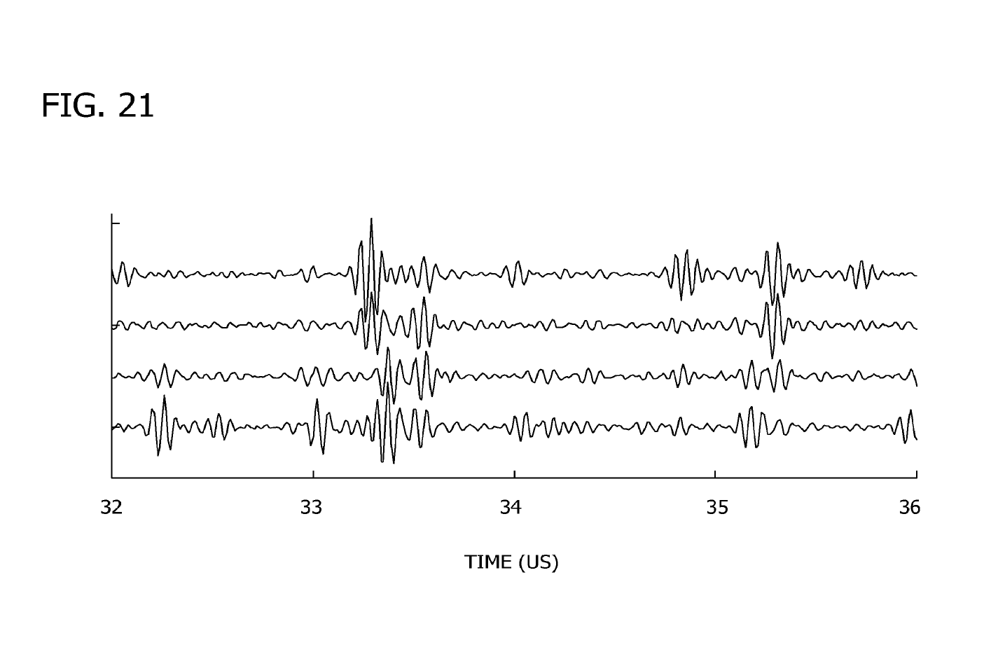

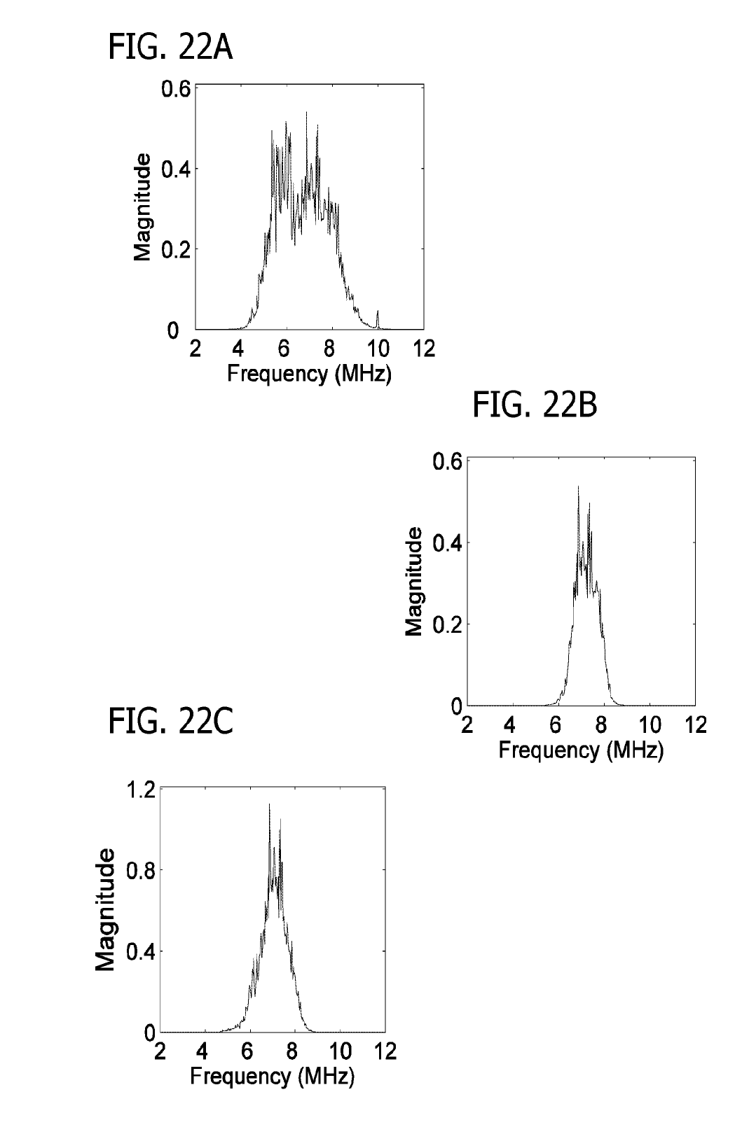

FIG. 21 shows segments of RF echo data obtained from adjacent scan lines (beam lines), which were positioned within the demarcated, enlarged region in FIG. 19. It can be seen that each scan line is composed of many ultrasound pulses each of which represents the echoed response of ultrasound wave when encountering a microbubble on the scan line. By examining the frequency spectrum (average of 68 beam lines in one B-mode image) of those echo signals, shown in FIG. 22A (no filter), we found there are two strong peaks at 6 MHz and 7.5 MHz, respectively. The 7.5 MHz peak came from the reflection of ultrasound by microbubbles, while the 6 MHz peak was probably caused by the introduction of noise or the interference between microbubbles.

A 3^{rd} order Butterworth band pass filter (6.5˜8 MHz) and the wiener filter were applied on the RF signals, respectively. The spectra of the RF signals after applying band-pass filtering is shown in FIG. 22B and after wiener filtering is shown in FIG. 22C. We found that both the band-pass filter and wiener filter can erase the effect of 6 MHz peak, however the resulted spectra are different: band-pass filtering simply deleted the signals with frequencies outside the defined band, but in the band, the spectrum remains unchanged; the wiener filter, however, tended to cancel the noise effect in the complete spectrum band, to recover the damaged signals to the greatest extent.

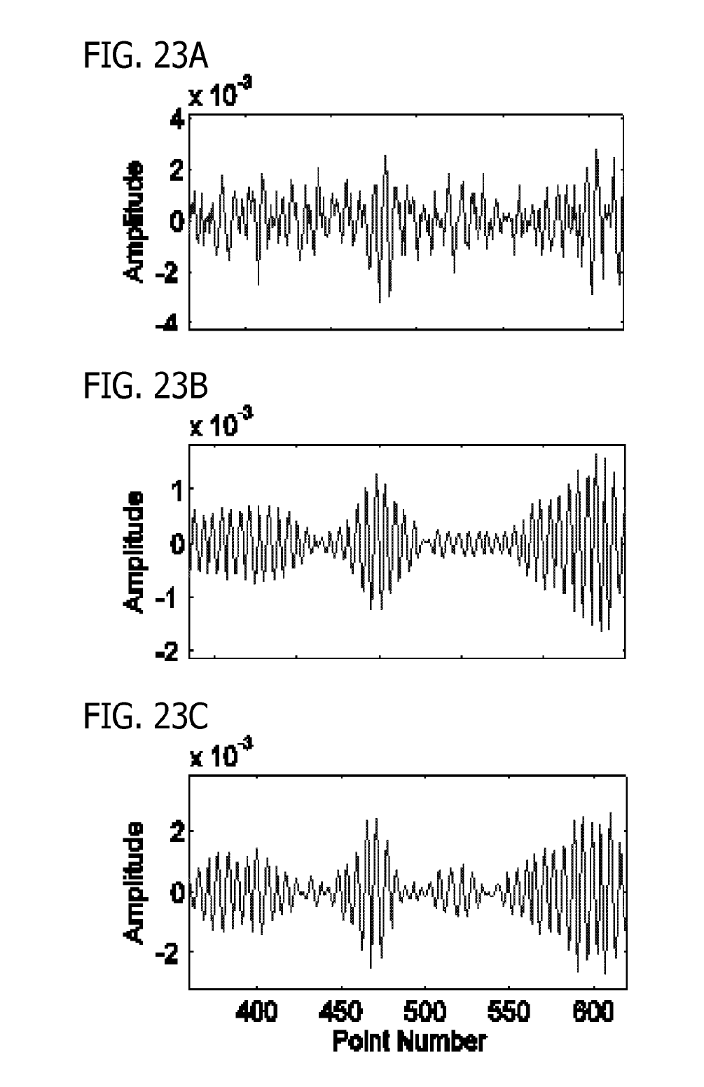

The differences can also be seen from the particle echo signals, as shown in FIG. 23. FIG. 23A shows a segment of RF data from one beam line in a B-mode image. It is noted that the noise is intermingled with the echoed pulses from bubbles; therefore it is impossible to reconstruct the bubble images on B-mode image. However, after the filtering, some bubble-reflected signals could be detected, as shown after band-pass filtering in FIG. 23B and after wiener filtering in FIG. 23C. By comparing 23B and 23C, we can also find the advantages of wiener filtering compared to band-pass filtering. First, the wiener filter can extract more bubble-reflected signals from original noisy signals. Second, for each bubble reflected signals, the recovered pulse by wiener filter has smaller temporal pulse length, which brings a better axial resolution of bubble images.

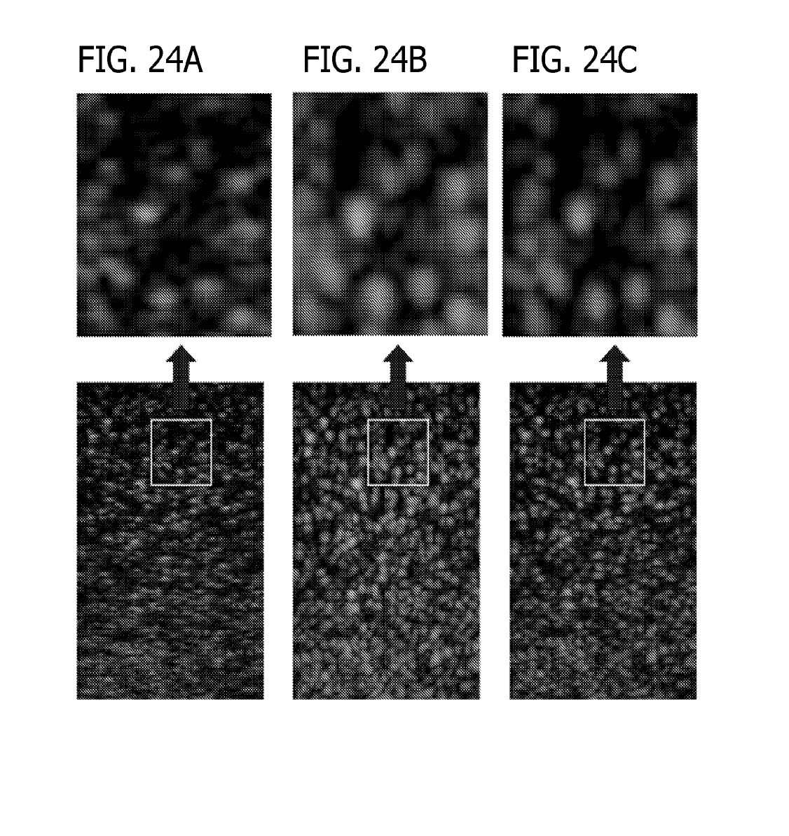

These could be further proved by comparing the particle images in FIGS. 24A, 24B and 24C, corresponding to FIGS. 23A, 23B and 23C, respectively. The bubble images from wiener filtering in 24C appear clearer and the background noise is more reduced. The sizes of bubble images are smaller and the blurring effect showing in images from band-pass filtering is not quite observed. After the filtering, the improvement on the low part of images in FIG. 24 is more obvious, where the microbubbles can be hardly recognized in original images in 24A.

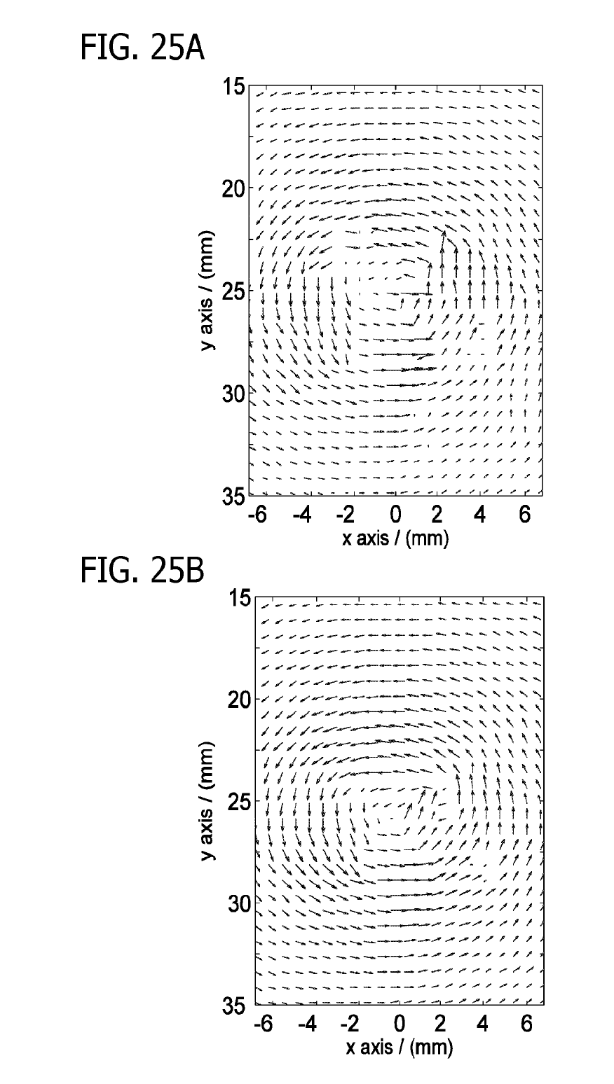

The improvement of the bubble images leads to the accuracy improvement of vector map obtained from cross-correlation, as shown in FIG. 25A, which are original image vector maps and 25B, which are maps after wiener filtering. It is noted that there are still some obvious outliers in the improved vector field, especially in the central region. Those outliers resulted from strong velocity gradients, which caused certain bubbles to move out of the imaging plane and other new bubbles to enter. Addressing such sources of vector error cannot be done in the pre-processing step, but can be performed by customized post-processing on the vector field, which will be discussed in the following section.

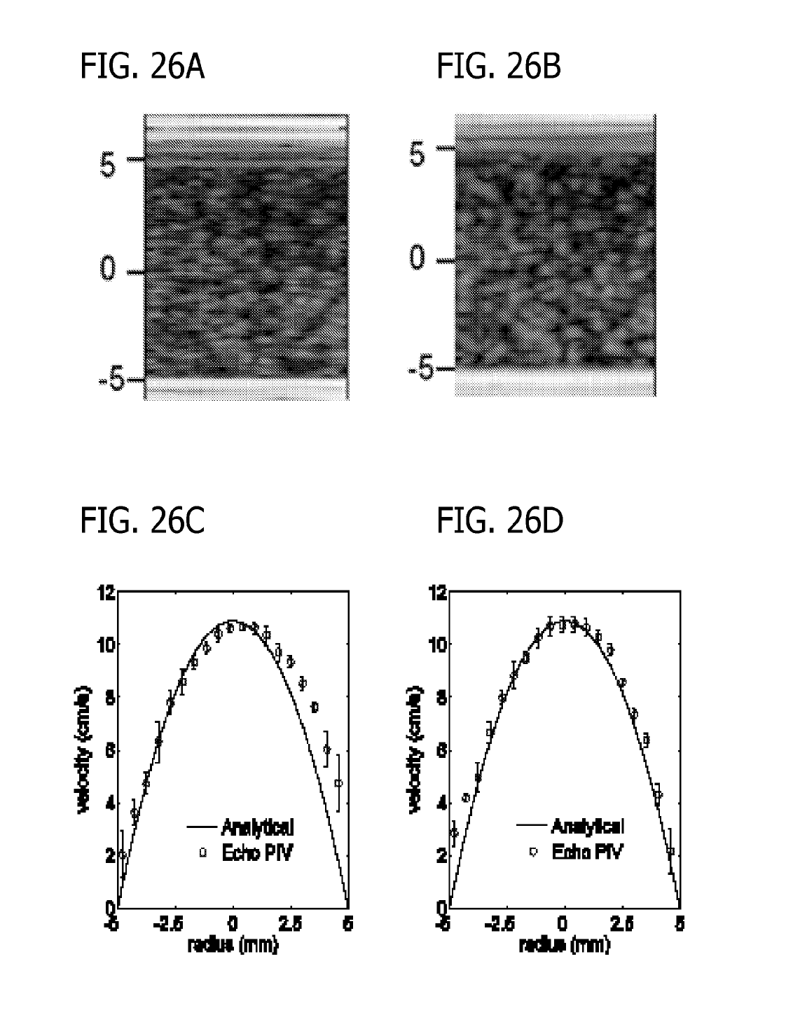

In order to quantitatively evaluate the performance of our wiener filtering method on improving the vector field, a laminar flow experiment was carried out. The tube diameter is about 10 mm with a flow peak velocity around 10 cm/s. The unprocessed and processed B-mode images are shown in FIGS. 26A and 26B, respectively. The experimental echo PIV results, obtained from 20 frames averaging, were compared with the analytical laminar flow velocities (cm/s), as shown in FIGS. 26C and 26D, of the unprocessed and processed images, respectively. Both the averaged velocity and standard derivation were improved, especially at the near-wall regions.

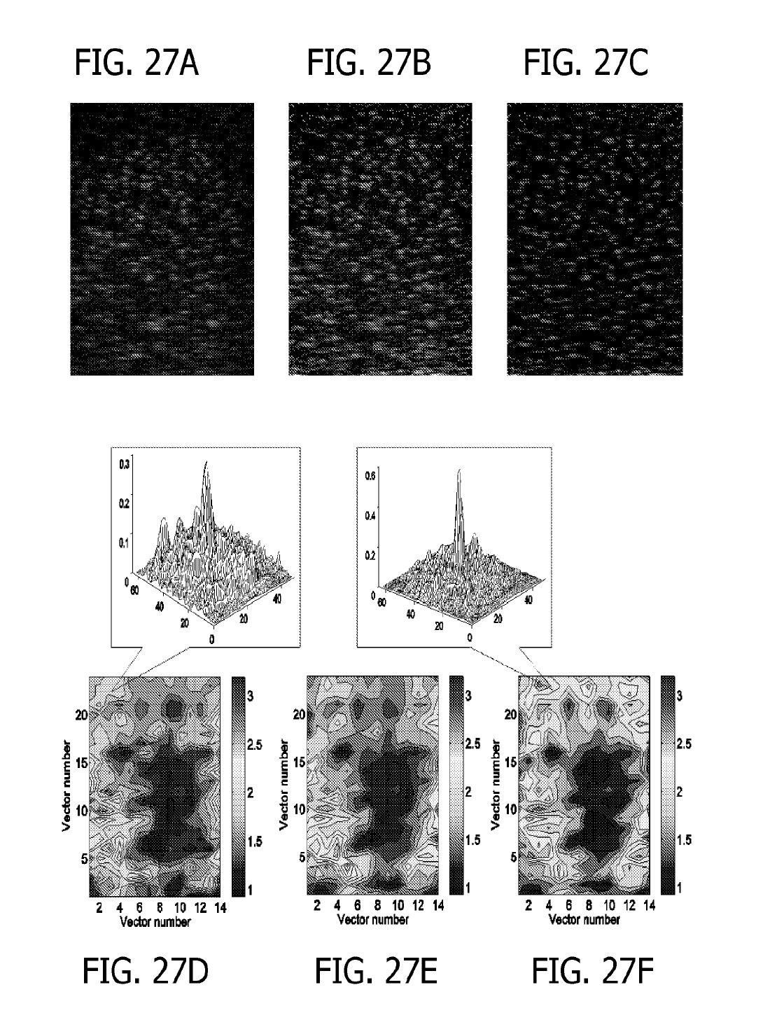

FIG. 27 illustrates B-mode images and SNR maps before and after correction of intensity non-uniformity of the invention: 27A, 27D illustrate the original image with SNR map; 27B, 27E after a max-min filter and 27C, 27F after a high-pass filter with SNR map.

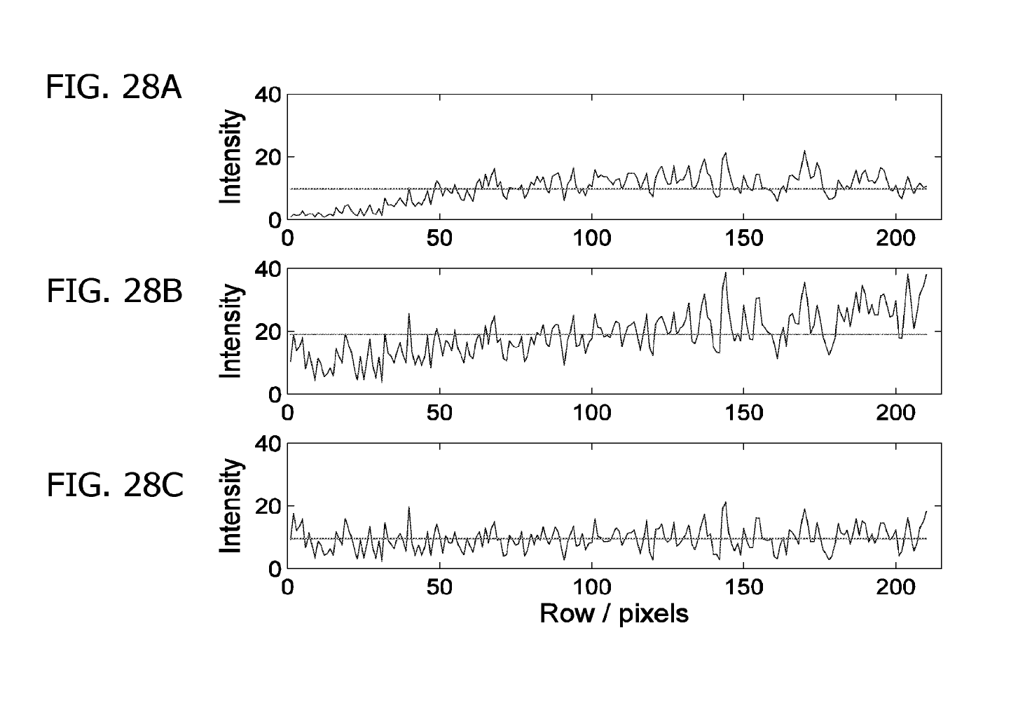

FIG. 28 illustrates the mean image intensity along columns relative to global mean intensity of the invention: 28A original; 28B after max-min filtering; 28C after man-min filtering and high pass filtering.

Echo Particle Image Reconstruction—Correlation-Based Template Matching (CBTM) Background

In Echo PIV method, the microbubbles are seeded in flows and traced by ultrasound B-mode imaging method, the successive microbubble images from which are cross-correlated to generate the velocity vectors showing the flow pattern. The initial in vitro studies showed the utility of this method in accurately measuring two-dimensional velocity vectors in a variety of opaque flows. Although this method appears promising, some issues are present. One of them is the high noise level of ultrasound images. Since Echo PIV method is based on correlation between ultrasound particle images, the signal to noise ratio (SNR) in images has great effect on the measurement accuracy of this method. Although there are a many filtering methods to improve the SNR of ultrasound images, such as the band pass filter, matched filter, inverse filter or deconvolution method, the echo particle images still remain a high noise level due to the strong interferences and nonlinear vibration of microbubbles. In one embodiment, a correlation-based template matching filter such as a filter employing correlation-based template matching (CBTM) is used in order to improve the SNR of echo particle images.

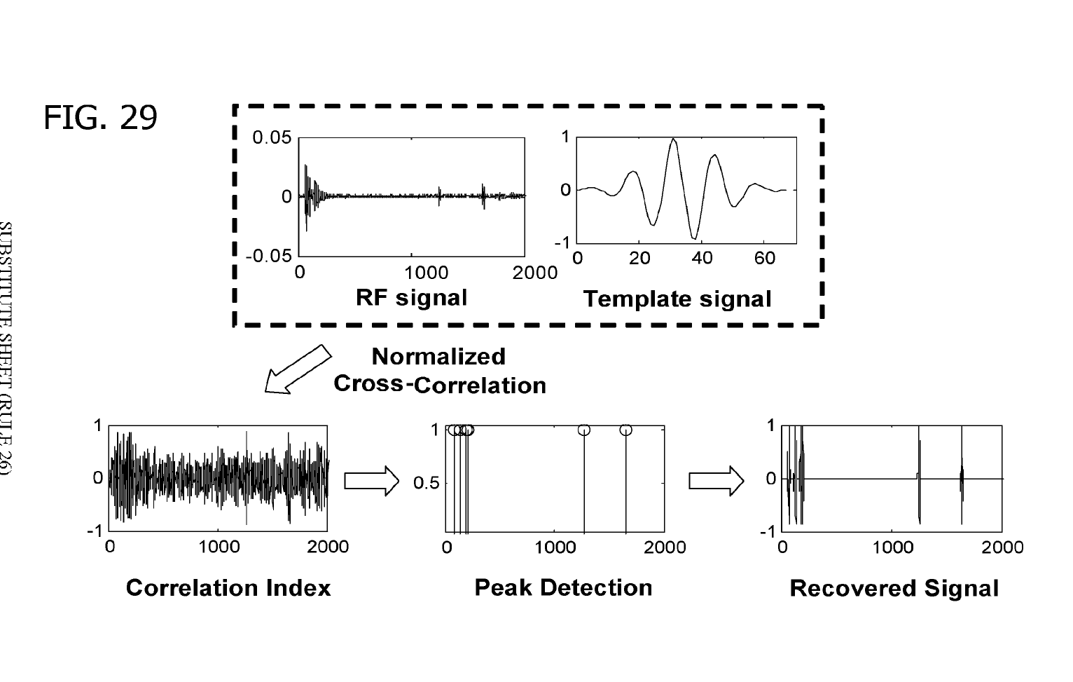

The correlation-based template matching (CBTM) method comes from the area of digital communication, in which a matched filter (convolution of a template signal with the received noisy signal) is used to detect the transmitted pulses in the noisy received signal. In the CBTM method, however, the cross-correlation between a template signal and the target signal rather than convolution is involved. The target signal is the particle echoed (RF) signal and the template signal is a standard Gaussian weighted pulse, as shown in FIG. 29. Considering the effect of signal amplitude on correlation index, a normalized cross-correlation is employed. The resulted correlation index is then peak-detected by assigning a threshold or range such that indexes over the threshold or within the range are indicative of signals corresponding to tracers. In this way, the particle position information is obtained, from which the improved pulse echo signal is generated by adding a standard single bubble echo signal to corresponding positions. During the peak detection, the threshold plays an important role in reconstructing the signals: if the threshold is too high, some particle information will be lost; with a low threshold, the “faked” particles will be incorporated into the particle images. The common range for echo particle images is between 0.8˜0.85 depending on the noise level in RF signals.

In the initial stage of one embodiment, we only take the standard Gaussian signal as a template, which does not consider any nonlinear effects of microbubbles. However, as another embodiment, in order to represent the particle echoed pulse more accurately, the measured bubble-echo signal could be employed.

Static Microbubbles Results

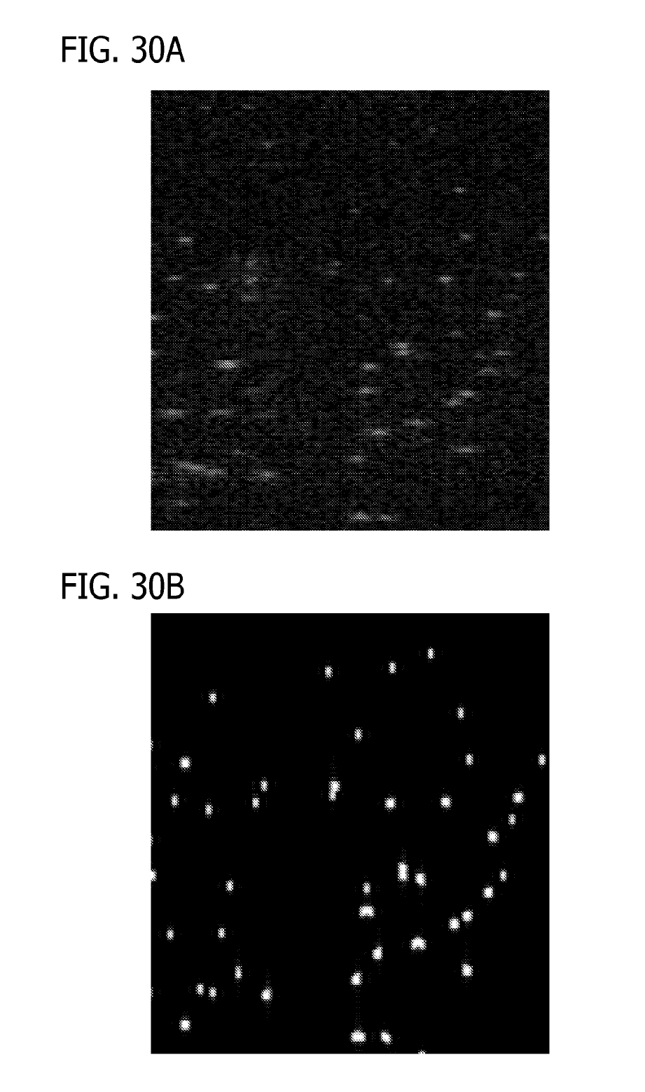

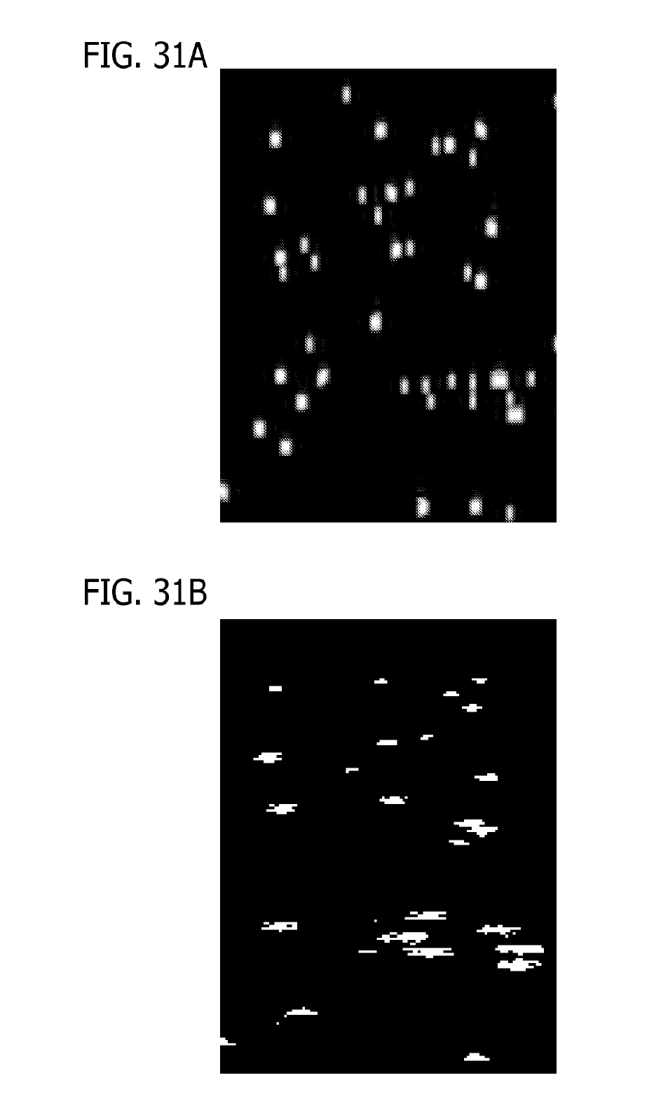

FIG. 30 shows the obtained echo particle images by using the traditional method and the CBTM method. In a traditional image (FIG. 30A) using B-mode construction, the particle images are blurred and the intensity is quite non-uniform. In contrast, the CBTM image (FIG. 30B) shows several important improvements, such as SNR enhancement, correction of non-uniform particle intensity, and regularization of particle shape and size. Also note that the CBTM method shows the ability to extract a single particle in the presence of other nearby scatterers, which will be better revealed in FIG. 31B (with CBTM) as compared to FIG. 31A (without CBTM).

Moving Microbubbles Results

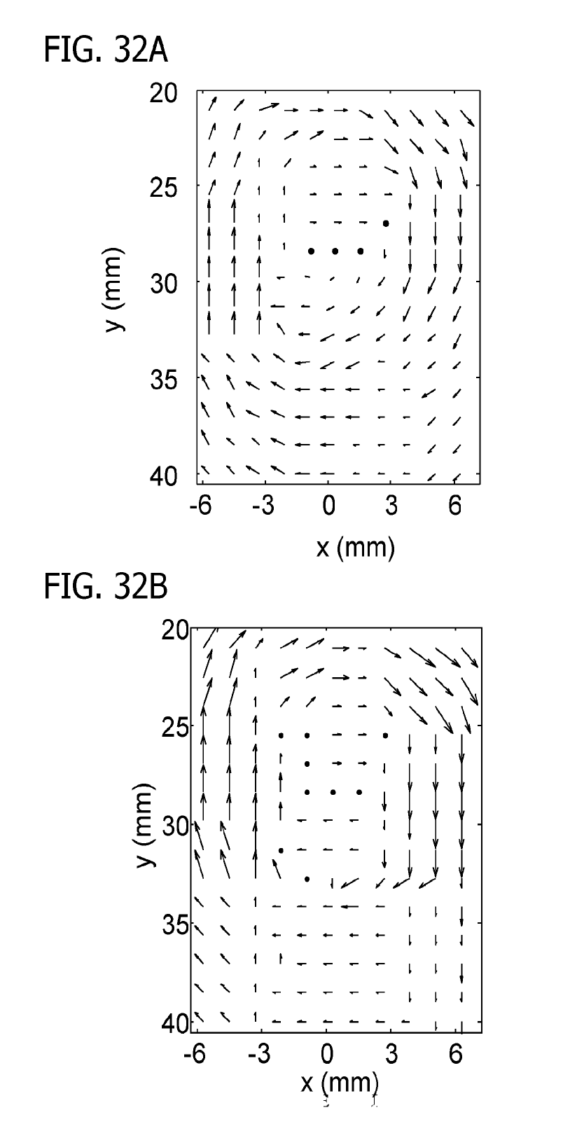

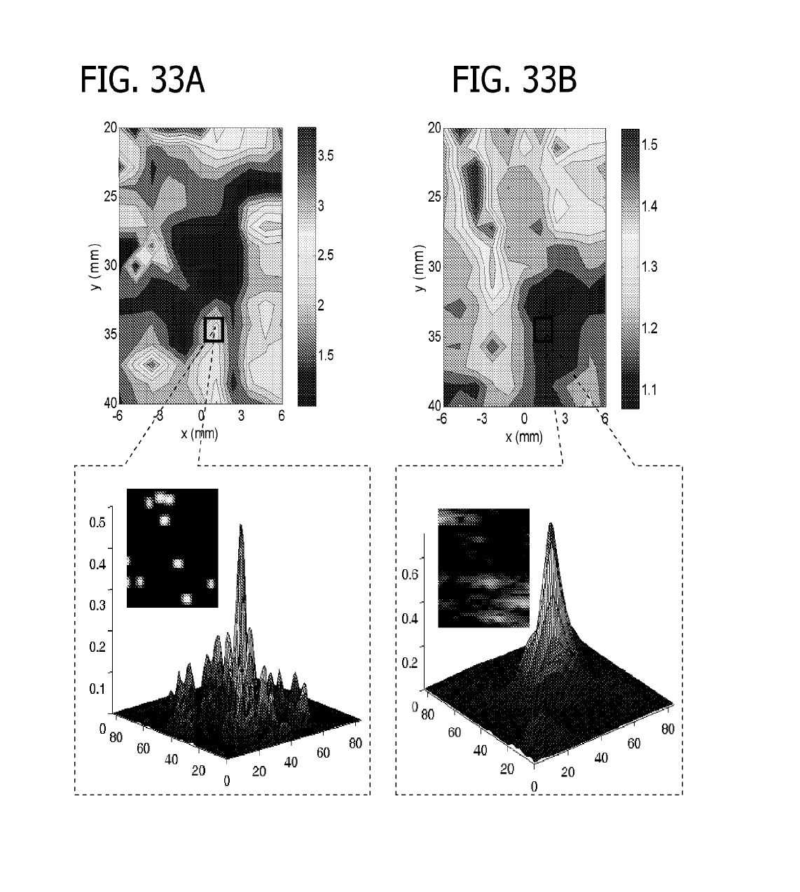

FIG. 31 shows the particle images recorded from a rotating flow experiment. The image without using the CBTM method is quite noisy and in the low-right region, the clustered bubbles could not be distinguished individually due to the limitation of transducer spatial resolution. The image is clearly improved by taking the CBTM method and more bubbles are identified from noise. This improvement brings the accuracy enhancement in velocity field measurement, as shown in FIG. 32A (with CBTM) as compared to FIG. 32B (without CBTM), especially in the low-right region where the original image is quite noisy. This can also be seen from the correlation-SNR map in FIG. 33A (with CBTM) compared to FIG. 31B (without CBTM). The low correlation-SNR region in FIG. 33B corresponds to the noisy region of particle image in FIG. 31B and a low accuracy of vector map in FIG. 32B. The improved vector field in FIG. 32A shows the effectiveness of the CBTM method in improving the echo particle images.

Not only does the CBTM method improve the SNR of particle images but it also allows identification of additional particles, which will further contribute to velocity measurement accuracy. In FIG. 33B (without CBTM), due to low particle number, the cross-correlation map is so broad that the correlation-SNR is low although the correlation index remains high. In contrast, in FIG. 33A (with CBTM), the improved particle image has more identified microbubbles, which produced a good correlation map with a narrow correlation peak and high correlation-SNR.

The accuracy of center velocity measurement in FIG. 32A (with CBTM) is not highly reliable since there are much fewer particles found in that region.

An CBTM method according to one embodiment of the invention is described to improve the echo particle images. The initial studies show its ability to enhance the SNR and spatial resolution of particle images, and the ability to reveal more particle information from noisy signals. A simple rotating flow experiment demonstrates the improvement of echo PIV measurement accuracy coming from the improved particle images.

Representative Templates Description

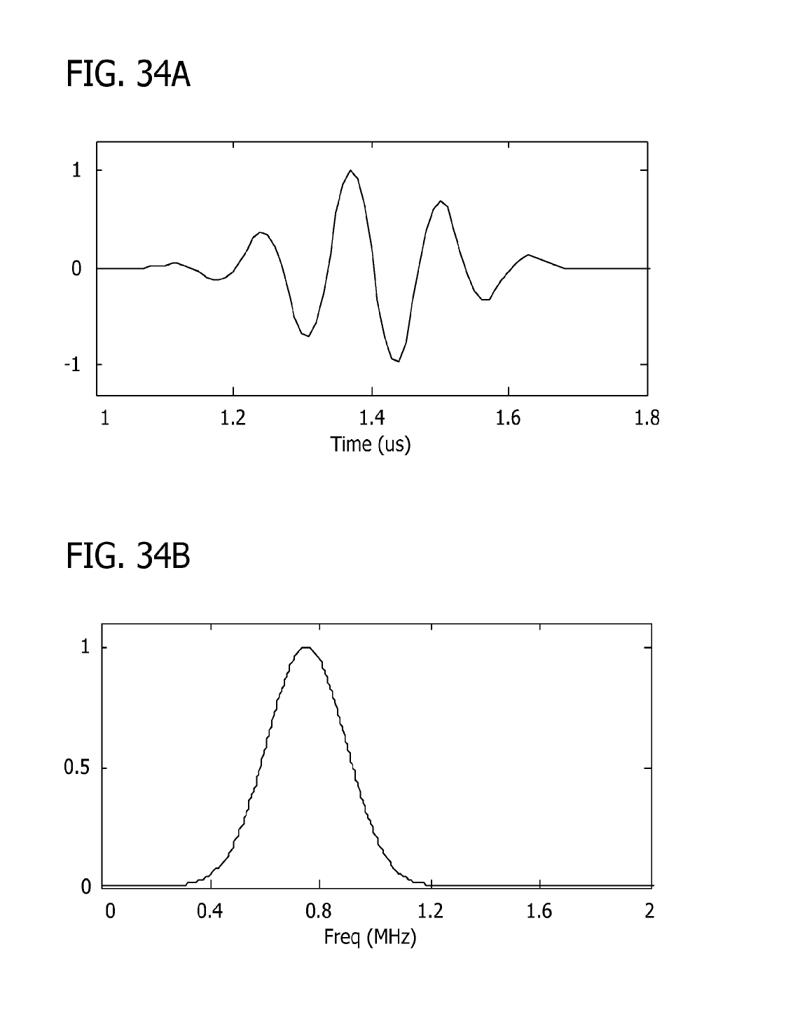

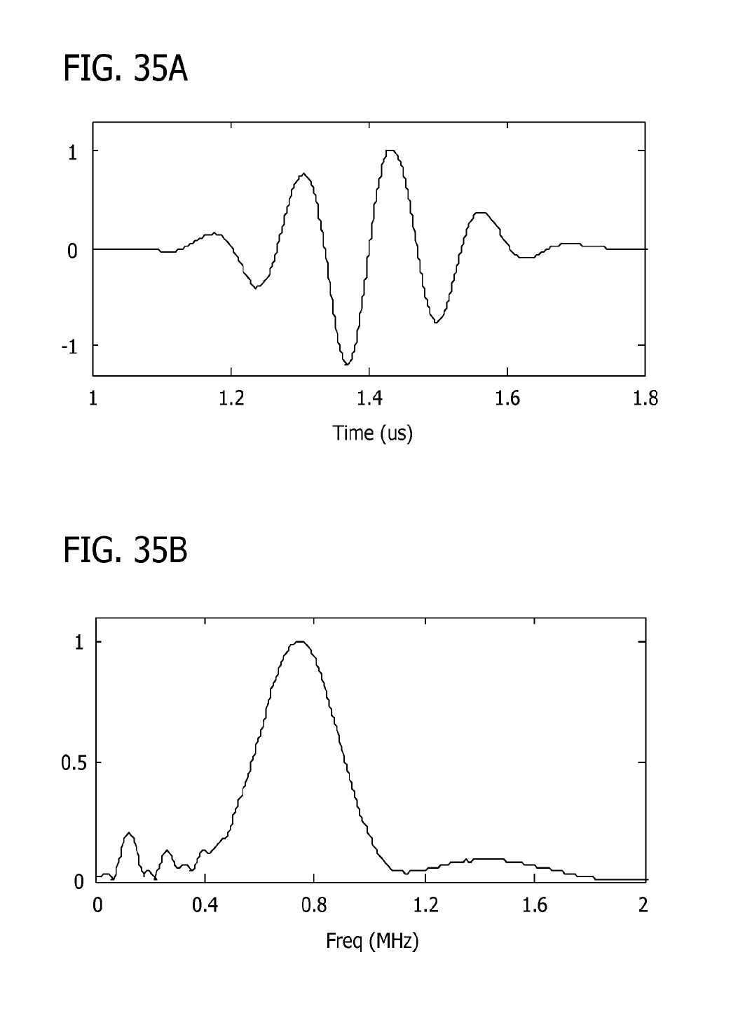

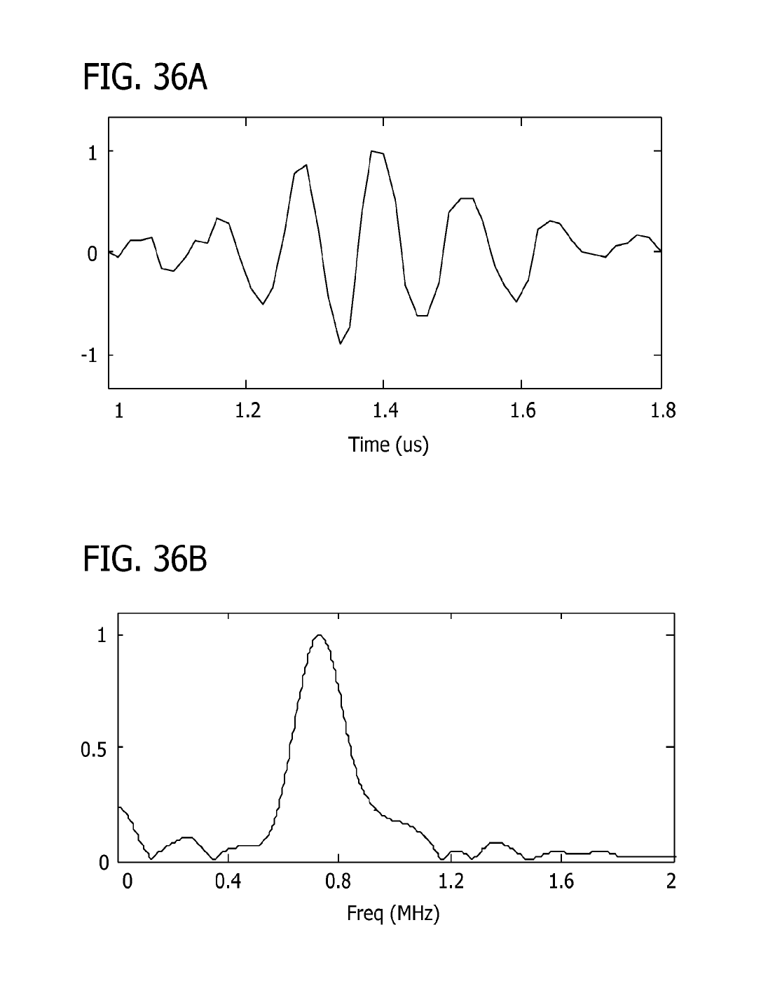

There are several types of templates that could be employed in our developed template matching filter. (The center frequency is 7.5 MHz).
a. A Standard Gaussian-weighted pulse as illustrated in FIG. 34, including: 34A time history and 34B frequency response. This template is a linear representation of bubble scatter.
b. A Simulated bubble-scattered pulse illustrated in FIG. 35, including: 35A time history and 35B frequency response. This template is a simulated bubble scatter by using the modified Rayleigh-Plesset (RP) equation, which allows consideration of bubble nonlinearity. This is shown in FIG. 35B.
c. A Measured bubble-scattered pulse illustrated in FIG. 36, including: 36A time history 36B frequency response. This template comes from the measured bubble scatter. The nonlinearity is also found from FIG. 36B.

Example of Convolution and Cross-Correlation Approach

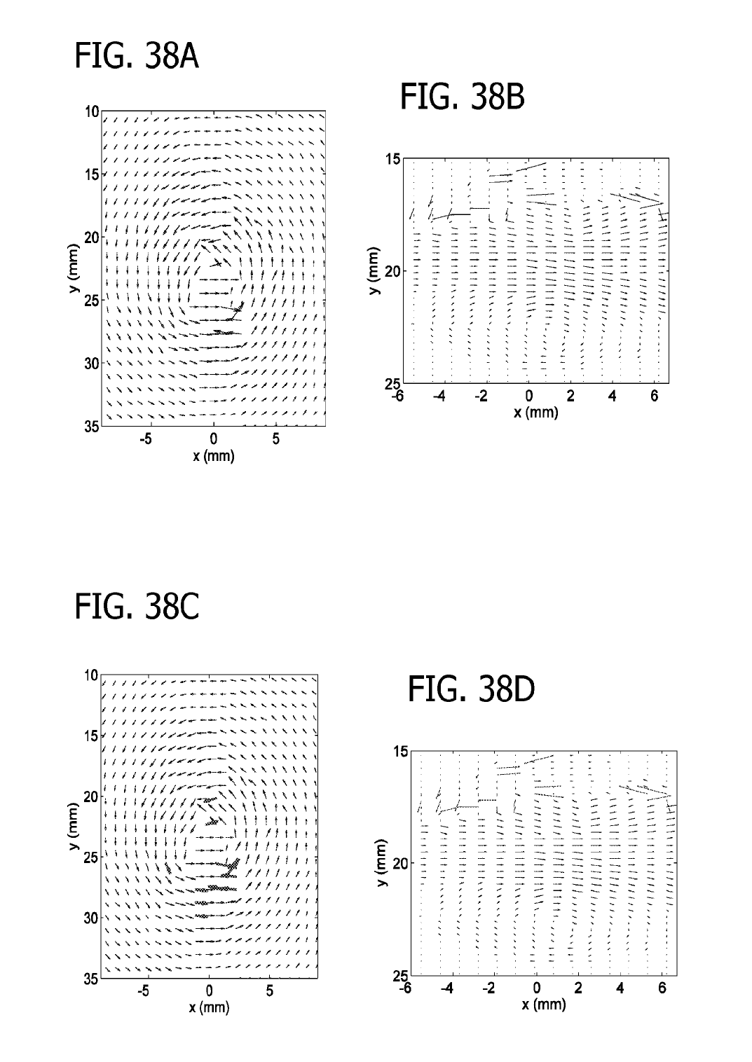

FIG. 38 illustrates vector maps from cross-correlation on B-mode image according to the invention: 38A shows rotating flow; 38B shows the aneurysm model; 38C shows the outlier identified by local filter; and 38D shows the outlier identified by global filter.

As noted above, the procedures of template matching by cross-correlation:
a. The normalized cross-correlation between the target signal and the template signal is applied, and a correlation index is obtained.
b. The correlation index is peak detected by thresholding, and the bubble positions are found from peaks.
c. The single bubble-scattered signal is accompanied with the bubble position information from peak detecting.

As noted above, a convolution operation is applied between the target signal and the template signal.

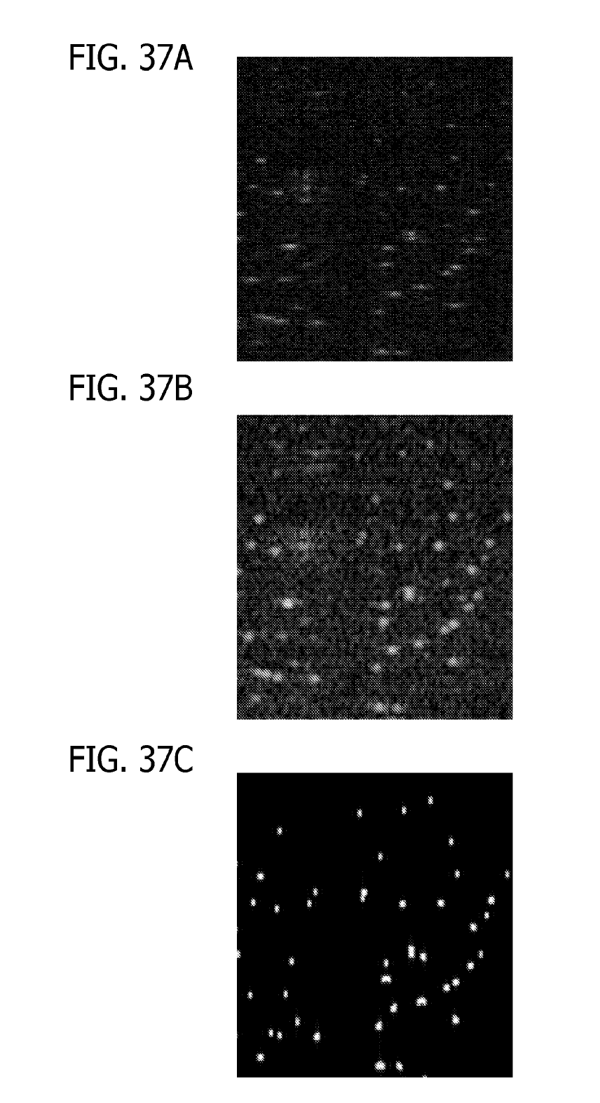

The images processed by template matching filter with convolution and cross-correlation are compared with the original image. Both convolution and cross-correlation can improve the bubble images. After the convolution method, the bubble images (FIG. 37B) themselves look good, but the background noise level is high. The correlation method can both improve the bubble images and increase the SNR of images.

Correction of Non-Uniform Intensity Distribution

Similarly to optical PIV, the ultrasound image also has a problem of non-uniform intensity distribution, which is mainly caused by the non-uniformity of the beam line. In the axial direction, the beam line is well focused at the focal region, introducing strong acoustic energy; however, at the near and far focal zones the energy is dispersed along the lateral direction. Such a B-mode particle image is shown in FIG. 27A. Typically in many situations, this is not a real problem. However, when the field of view (FOV) is large so as to cause the low intensity of microbubble images at the near and far focal regions, the low SNR ratio at those regions (since the intensity of microbubble images is low, comparable to noise level) will influence the accuracy of cross-correlation. As in optical PIV, a max-min filter was employed to solve this problem. By examining the mean image intensity along the column, however, we found the max-min filtering method could not solve this problem completely, as shown in FIG. 28; therefore, a high-pass image filtering method was used after the max-min filtering. In order to show the influence of the intensity correction on SNR, the cross-correlation was conducted and the SNR, ratio of the maximum correlation peak value to the second highest correlation peak value, was computed for each interrogation window. The SNR maps are shown in FIG. 27D-27F. It is clear that after the max-min and high pass filtering, the SNR is improved significantly, from 1˜2.2 in 27D to 1.1˜3.5 in 27F. The improvement of SNR will increase the probability of finding the accurate peak in the correlation map, so as to increase the correlation accuracy to some extent. However, it is also necessary to note that whatever methods are employed, the SNR and vector map in center region cannot be improved due to the high velocity gradient and microbubble pattern distortion in an interrogation window. The vector map could be smoothed by detecting incorrect vectors and replacing them with the interpolated values, which will be discussed in the next section.

Post-Processing: Improvement of Vector Field

The task of post processing is to increase the velocity measurement accuracy, the dynamic velocity range and the velocity map resolution. In the past twenty years, there are numerous studies in conventional or digital PIV areas, which significantly improved the quality of the resultant velocity vectors. However, these have relied on the high quality image sources seen in optical imaging. What we are interested in is how and how much the echo PIV method and the echo PIV vector field can be improved. In this section, we adapted the previous PIV methods into our echo PIV methods, in order to systematically investigate the factors influencing the improvement of echo PIV velocity map, and demonstrate the possibility of improving this method, using by way of example, several types of flow, such as rotating flows, tube flows and flows through abdominal aortic aneurysm (AAA) models.

Improvement of Velocity Vector Accuracy

As discussed in the previous section, the pre-processing on B-mode images can improve, to some extent, the measurement accuracy. However, there are still some obviously incorrect vectors remaining in the vector field, introducing errors to the measured flow velocities. To eliminate those outliers, the vector field is smoothed by numerous customized neighborhood filters, which are followed by interpolation methods.

FIGS. 38A and 38B show two vector maps from two experiments, a rotating flow model and an aneurysm model, respectively. The discontinuities in these two maps are obvious: in the rotating flow map, there are certain outliers that are quite different from their neighbors; similarly, at the upper vortex flow of the aneurysm model, some values are impossibly large. These types of outliers are respectively due to local errors and global errors. Based on the assumption that in the real flow field, the velocity difference between the neighboring velocity vectors is smaller than a certain threshold, global and local filters are designed in order to eliminate these two types of outliers (see Appendix B for details); the outlier identifications are shown in FIGS. 38C and 38D, respectively. By appropriately adjusting the thresholds, these filters will generate fewer outliers.

Although some outliers could be found by using global and local filters, it is obvious that these two filters are not enough to detect all of the inaccurate vectors. Generally, the generation of outliers is quite related with the SNR of the cross-correlation map, thereby a SNR filter is designed to identify those vectors with a noisy correlation map. The design details are expanded upon in Appendix B.

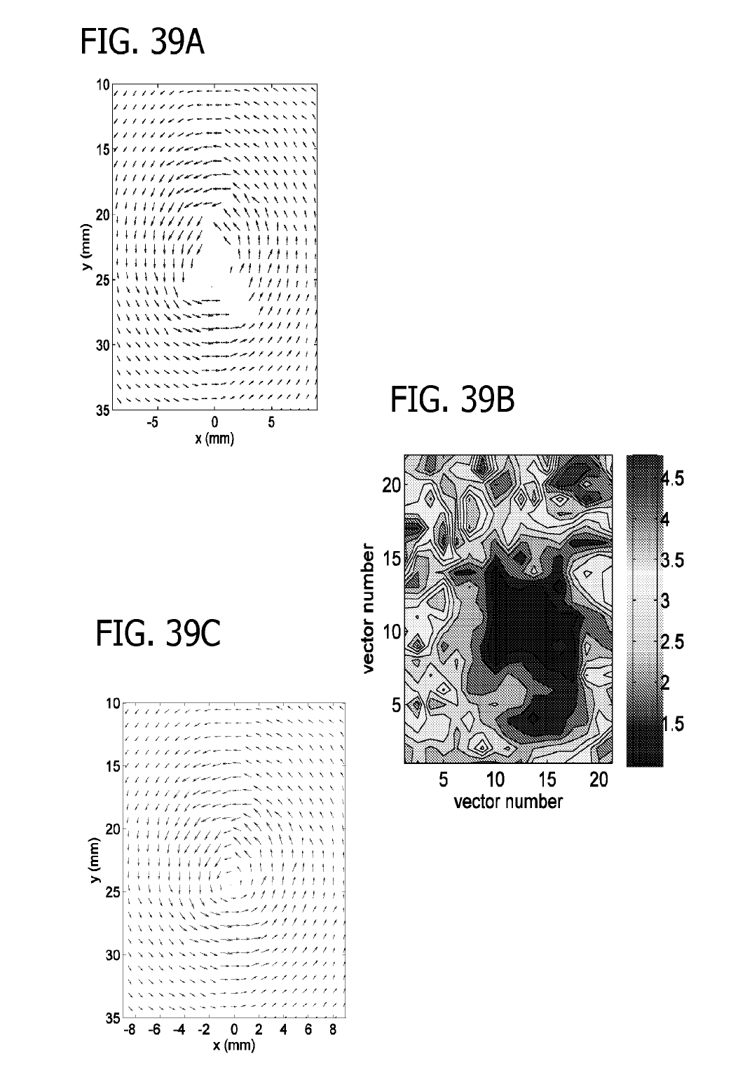

FIG. 39 shows the improvement of vector map from rotating flow: 39A shows outliers deleted by SNR filter; 39B shows SNR map from cross-correlation; 39C shows interpolated vector map from 39A.

Enhancement of Velocity Field Resolution

The accuracy of the cross-correlation of two images depends on many factors for echo PIV, including the spatial resolution of two images, the noise level, the bubble concentration, the gradient of velocity, and also the interrogation window size. Among all of the factors, the interrogation window size is quite important for the quality of cross-correlation, since the detection of either a valid or a spurious vector directly depends on the number and distribution of the particle images inside the interrogation area. For Echo PIV images, generally it is not completely possible to find 10 particle images and four matched image pairs in each interrogation window, since a low bubble concentration is required. The larger interrogation window size brings more particle image pairs, thereby the accuracy of cross-correlation is improved to some extent. However, this is not always true. In the regions with large velocity gradients, the local velocities in differing directions may appear as a small average or even no velocity with a large standard derivation. In such cases, velocity measurement accuracy generally deteriorates. On the other hand, the window size should be as small as possible in order to maximize the spatial resolution of the vector field. However, decreasing window size too far will not provide enough image pairs in each interrogation area; therefore the quality of the vector map will also deteriorate. Thus, the optimal window size arises from a tradeoff between generating sufficient vector accuracy and sufficient vector field resolution.

The effects of different interrogation window sizes on vector resolution and vector accuracy were investigated by employing the rotating flow model, in which both optical PIV and echo PIV were measured simultaneously in order to validate the results of echo PIV.

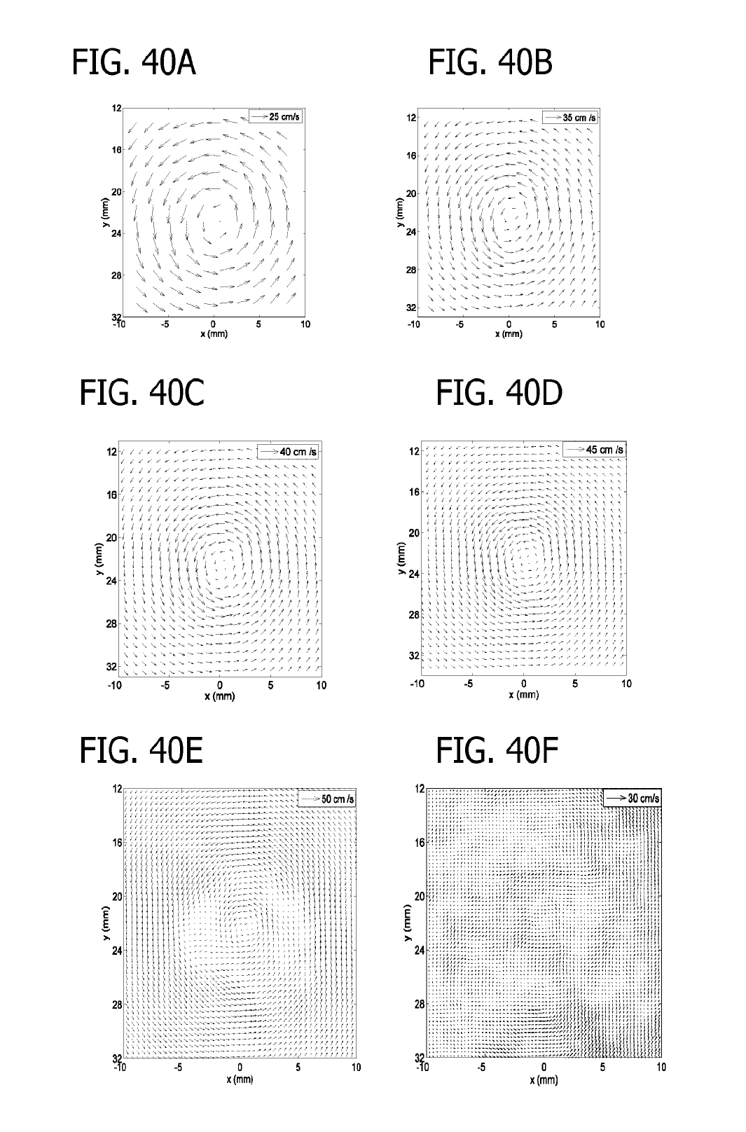

FIG. 40 illustrates a vector field maps of a rotating flow for different window sizes according to the invention, from an average of 10B-mode images: 40A shows 56×56, 40B shows 40×40, 40C shows 32×32, 40D shows 24×24, 40E shows 16×16, and 40F shows 8×8. The size of the cross-correlated B-mode particle images is about 180×160 pixels, and one pixel displacement represents about 5 cm/s in velocity. The maximum velocity in this rotating flow is around 30 cm/s. It is noted that for an interrogation window size larger than 24x24, the vector maps look quite consistent. When a 16×16 window size is used, the vector map still looks good at the outer regions, but has many incorrect vectors at the center where large velocity gradients exist. Lastly, for the 8×8 window size, the vector map deteriorates substantially. The reason is that the maximum reliable displacement from cross-correlation is approximately one fourth of the interrogation size. Therefore, when the interrogation window size is 16×16, only up to 4-pixel displacement can be accurately measured, which corresponds to a velocity of up to 20 cm/s. Similarly, only velocity up to 10 cm/s could be accurately measured for a window size 8×8.

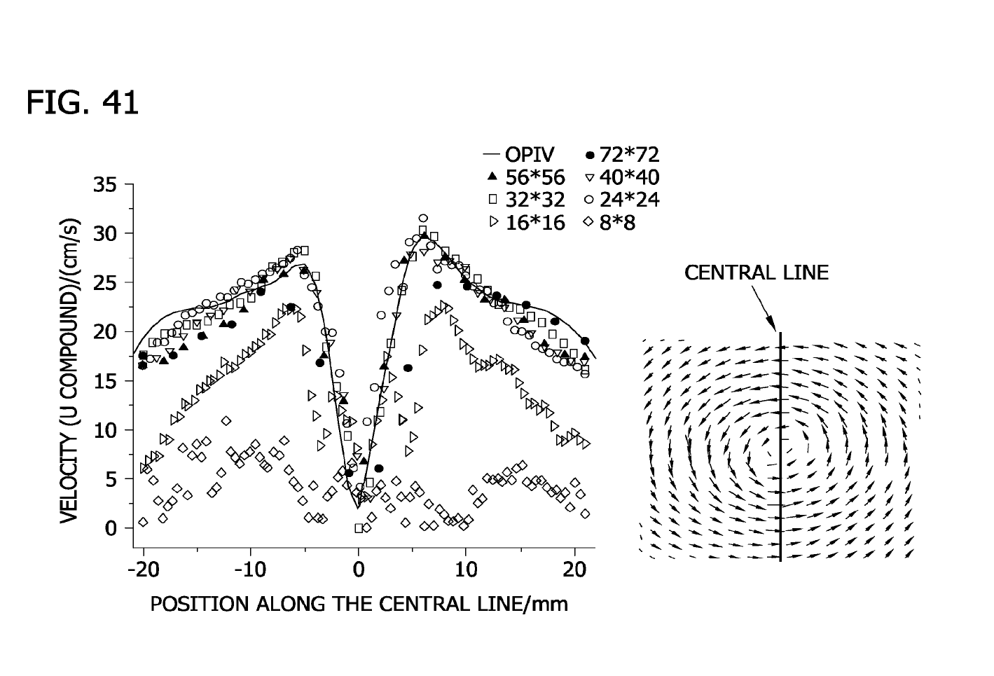

FIG. 41 compares the results of the optical PIV and echo PIV for different interrogation window sizes. The echo PIV results are the average of 14 groups of data, with stand derivations shown in FIG. 42. Optical PIV (e.g., TSI System, Shoreview, Minn.) data were obtained by seeding the rotating flow with polystyrene microspheres (diameter: 100-500 μm). Only the horizontal component of velocities along the vertical central line (shown as in lower-right corner of FIG. 41) was compared here, since the vertical component along that line is quite small. FIG. 41 further strengthened our conclusion that an optimal interrogation window size exists for the tradeoff between measurement accuracy and vector field resolution. When a larger window size is selected, such as 72×72 and 56×56, although the vector maps look good, the accuracy is degraded when compared to optical PIV results, because the velocities at large gradient regions (e.g. in the center of flow) are averaged due to the large window size; therefore, the maximum velocity is lower than that seen in Optical PIV. For the small window size, such as 16×16 and 8×8, the measurement values deviate significantly from optical PIV results due to the bad cross-correlation caused by low number of bubbles in each interrogation window.

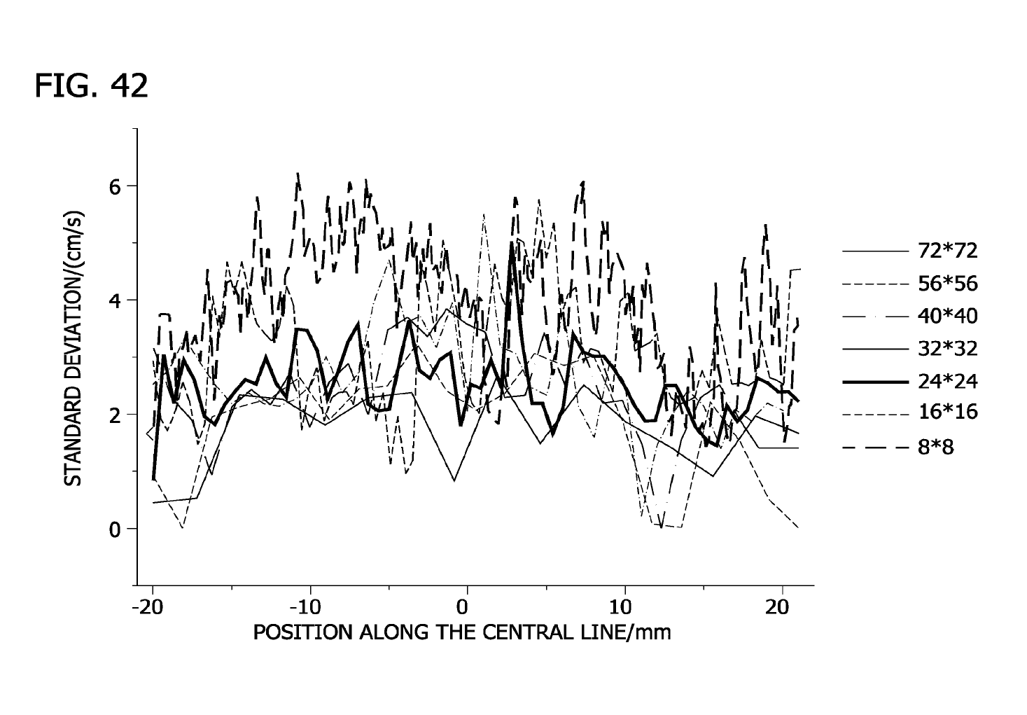

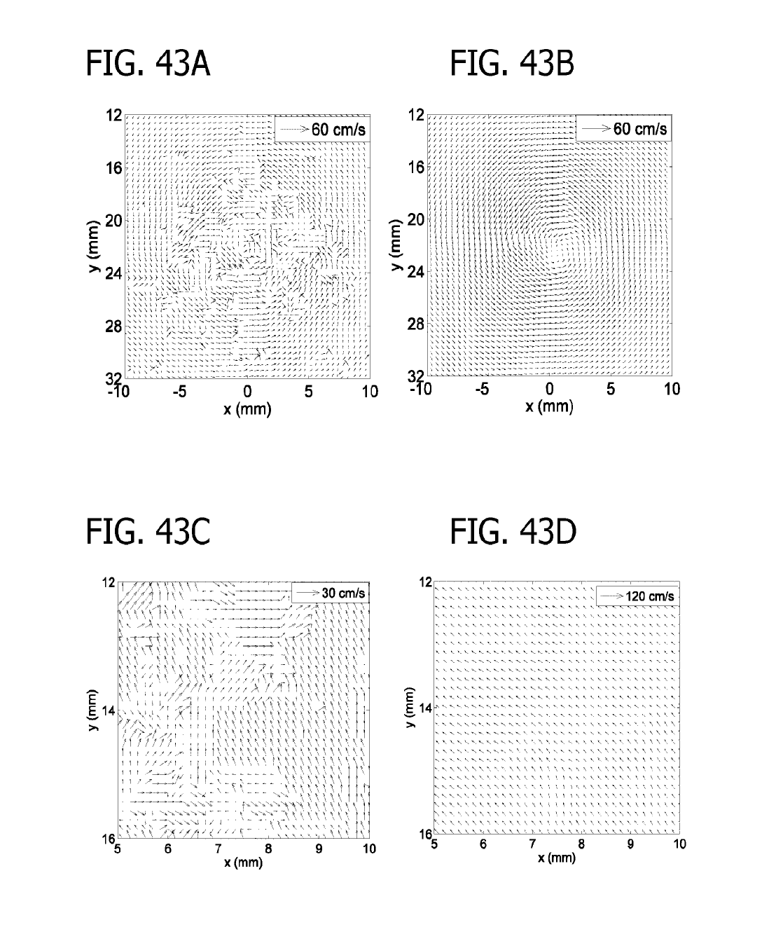

Since the conventional PIV analysis could not improve the resolution of vector field while maintaining good measurement accuracy, advanced methods are introduced. The adaptive window size and discrete window offset algorithms, commonly used in PIV techniques but adapted to account for the higher SNR and lower resolutions of ultrasound imaging, are employed to improve both the accuracy and resolution of our echo PIV method. The results are shown in FIG. 43. FIG. 43 illustrates a comparison of vector maps by using discrete window offset and conventional cross-correlation with small window sizes, according to the invention: 43A shows 16×16, DWO; 43B shows 16×16, SCC; 43C shows 8×8, DWO; and 43D shows 8×8, SCC. After using the discrete window offset (DWO), the vector maps improved significantly compared to results from standard cross-correlation (SCC) even when a window size of 16×16, or 8×8 (only a partial map is shown) is used. Therefore, we conclude that the resolution of echo PIV method can be greatly enhanced when advanced PIV analysis are employed.

Improvement of Dynamic Velocity Range

Another important criterion to evaluate a velocimetry method is the dynamic velocity range (DVR). The DVR is defined as the ratio between the maximum measurable velocity range and the minimum resolvable velocity measurement. For the conventional cross-correlation algorithm, the ratio is determined by the interrogation window size. For example, if a 32×32 pixel window size is selected, the maximal displacement that could be correctly measured is around 4˜5 pixels (about one fourth of window size), and the minimal displacement should be one pixel. So the DVR is about 4˜5, which is too low for a velocimetry method. However, if using peak-interpolation schemes in the correlation plane, sub-pixel accuracy can be obtained. Therefore, the DVR could be enhanced to a value about 40˜50. This DVR is generally sufficient for medical in vivo applications.

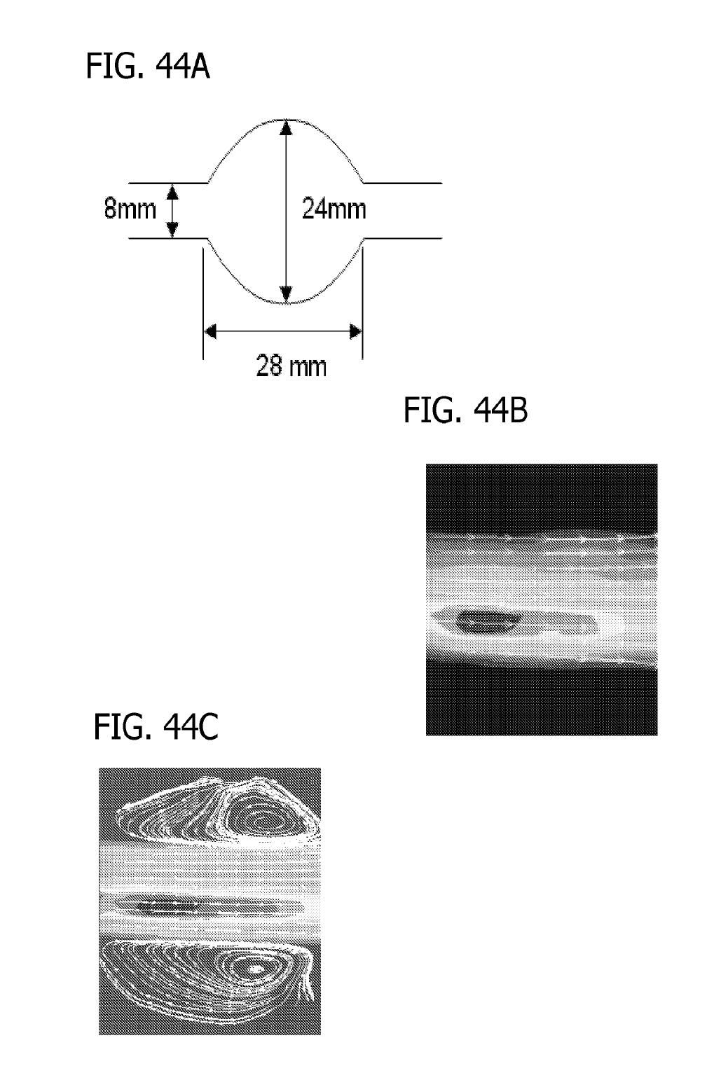

Using by way of example flow through the aneurysm model, we compared the DVRs before and after application of the sub-pixel interpolation algorithm. FIG. 44A shows the dimensions of aneurysm model and the results are shown in FIGS. 44B and 44C before and after sub-pixel interpolation, respectively. In this experiment, the peak velocity value in the center of aneurysm is around 11 cm/s, however, the velocities in the dilated parts range between only 0.1˜1 cm/s. Without the sub-pixel interpolation, the conventional cross-correlation failed to measure the low velocities since the dynamic velocity range is too low. After the sub-pixel interpolation, it is clearly shown that the velocity distributions in the dilated parts were obtained.

Example of Echo PIV Measurement on Abdominal Aortic Aneurysm Models

Abdominal aortic aneurysms (AAAs) are localized balloon-shaped expansions commonly found in the infrarenal segment of the abdominal aorta, between the renal arteries and the iliac bifurcation. Abdominal aortic aneurysm rupture has been estimated to occur in as much as 3%-9% of the population, and represents the 13th leading cause of death in the United States, producing more than 10,000 deaths annually. Thus, determining the significant factors for aneurysm growth and rupture has become an important clinical goal. From a biomechanical standpoint, AAA rupture risk is related to certain mechanical and hemodynamic factors such as localized flow fields and velocity patterns, and flow-induced stresses within the fluid and in the aneurysm structure. Disturbed flow patterns at different levels have also been found to trigger responses within medial and adventitial layers by altering intercellular communication mechanisms. Thus, localized hemodynamics proximal, within and distal to AAA formations play an important role in modulating the disease process, and non-invasive and easy-to-implement methods to characterize and quantify these complex hemodynamics would be tremendously useful. Echo PIV measurement on abdominal aortic aneurysm models.

Experimental Methods

The custom-designed Echo PIV system was applied to in vitro fusiform AAA models under steady flow conditions. A centrifugal pump was employed to circulate water through a plastic aneurysm model between two containers with a fixed head differential, with flow rate adjusted between 0.2-0.6 L/min using a shunt valve. The aneurysm model was embedded in a tissue phantom. It has non-dilated inlet and outlet tubes with diameter of 8 mm and length of 200 mm, and has a bulge with length of 28 mm and a maximum diameter of 24 mm. The ultrasonic transducer was well aligned so that the scan plane coincided with the vessel centerline. Ultrasound contrast microbubbles (Optison®, Amersham, UK) were used as flow tracers and seeded into the flows. B mode particle images were acquired by the system at a frame rate of 150 fps with a focal depth of 24 mm and FOV of 40 mm (depth) by 27 mm (width).

At the same time, a 3D computational aneurysm model with similar dimensions was constructed by using SOLIDWORKS (Solidworks Corp., MA). To maintain a well developed flow before the entrance to the aneurysm region and minimize the flow disturbance downstream, we made the vessels proximal and distal to the aneurysm as long as 150 mm with a diameter of 8 mm. The maximum diameter of the aneurysm was 24 mm and it was smoothly translated to straight vessels using fillet at conjunction areas. This solid model was then imported into ICEM-CFD (ANSYS Inc., PA) and meshed by using 35,000 hexahedral elements. Detailed mesh distribution is presented in FIG. 45. A steady state computational fluid dynamics analysis was performed so that we can compare the simulation results with in vitro measurements. Note that laminar flow was assumed due to the low flow velocity used, and constant velocity and constant pressure was used at the inlet and outlet respectively.

Results

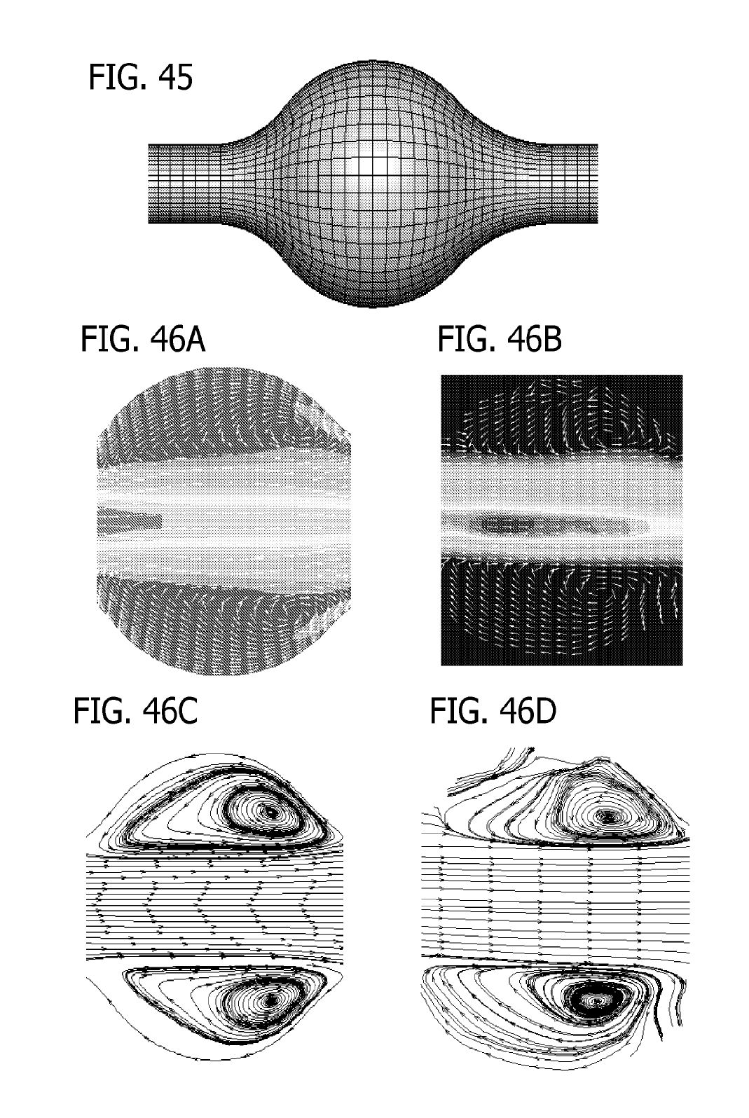

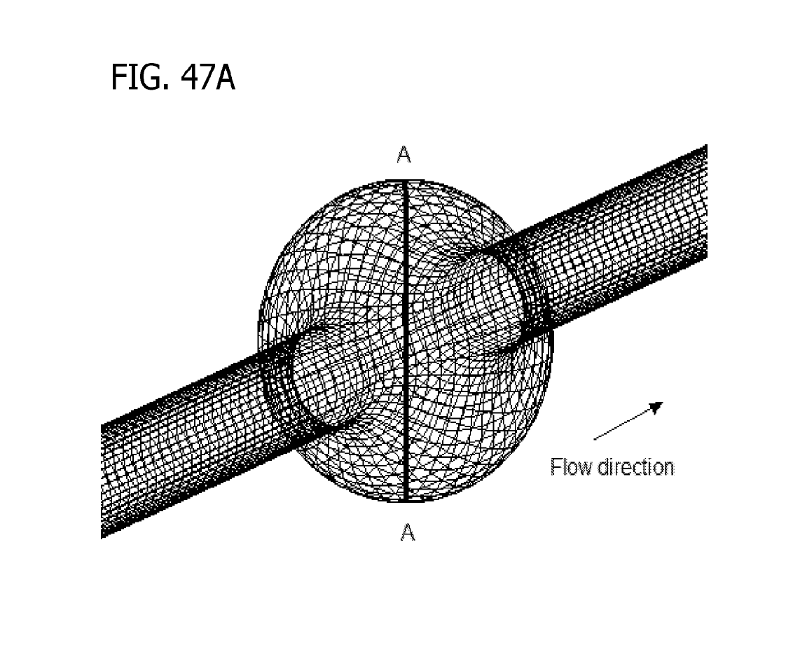

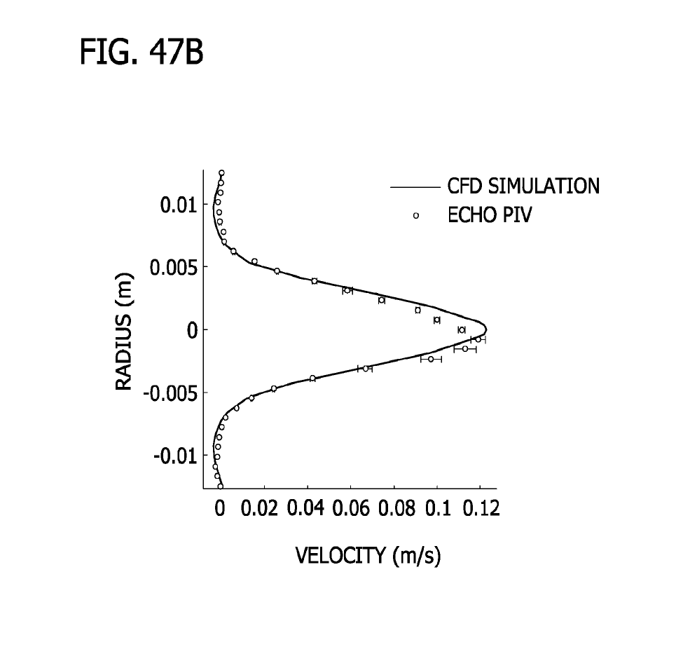

B-mode images were constructed from the acquired RF data for the flows in AAA model and a cross correlation was performed on these images to calculate the local velocities in the flow field. FIGS. 46A and 46B show the CFD simulated and Echo PIV measured velocity vectors within the AAA model under steady flow conditions. Note that in these two FIGS. 46A and 46B, the magnitude of velocity is denoted by the depth of color but not the length of velocity vector since the flow field in the AAA model has high velocity gradients. If we express the velocity magnitude by the length of velocity vector, many vectors, especially those close to the arterial wall and vortex ring, may become invisible because of their small amplitudes. Thus, in FIGS. 46A and 46B, the velocity vectors have equal lengths and reveal clearly the flow patterns inside the AAA model. FIGS. 46C and 46D show the streamlines derived from the CFD simulation and Echo PIV measurement of velocities. To verify the performance of the Echo PIV system, we also extract the velocity profile along line A-A from the CFD simulation (see FIG. 47A), which corresponds to the transducer scan line that goes through the center of the AAA bulge (where the AAA model has the maximum diameter) in experiment, and compare it with the velocity profile derived from measured results. FIG. 47B shows the comparison of the velocity profiles from CFD simulation and Echo PIV measurement, in which the Echo PIV profile is the average result calculated from seven consecutive B mode image pairs.

Transducer and Advanced Transducer Driving Methods for Echo PIV

For peripheral vascular imaging using Echo PIV, such as carotid artery imaging, the diameter of the vessel is about 0.5-1 cm, the maximum blood velocity is about 1 m/s and the imaging depth is usually less than 5 cm. To obtain clear images of blood vessel boundaries and contrast agents, high frequency transducers (center frequency around 10 MHz) are preferred to achieve good spatial resolution. Also, the transducer bandwidth should be large 70%) so that advanced imaging methods, such as 2^{nd} harmonic imaging, sub-harmonic imaging and ultra-harmonic imaging can be employed to maximize the bubble detection in tissue structure.

Tissue is relatively incompressible and responds linearly to ultrasound. The non-linear behavior of ultrasound contrast agents cause the highly compressible bubbles to act as active scatterers with significant components in the sub and higher harmonics of the incident frequency. Examination of the harmonic frequencies can thus separate the echo signal due to microbubbles from that due to tissue. Broad bandwidth transducers can be operated efficiently in a large frequency range thus allow the receiving of the sub or 2^{nd} harmonic content of the transmitted ultrasound pulses to improve the bubble detection.

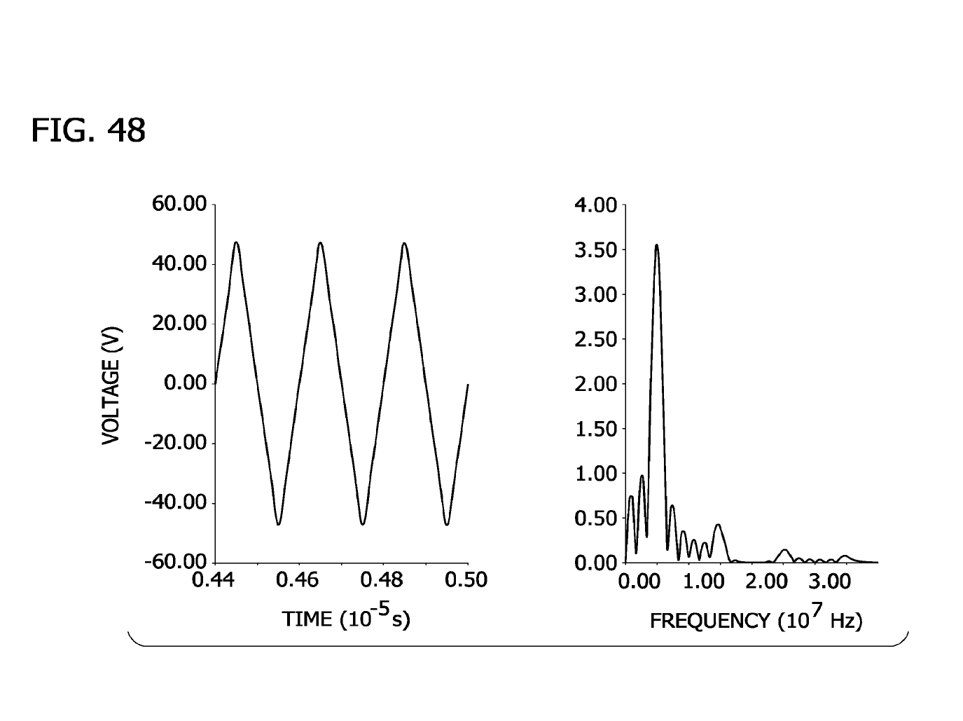

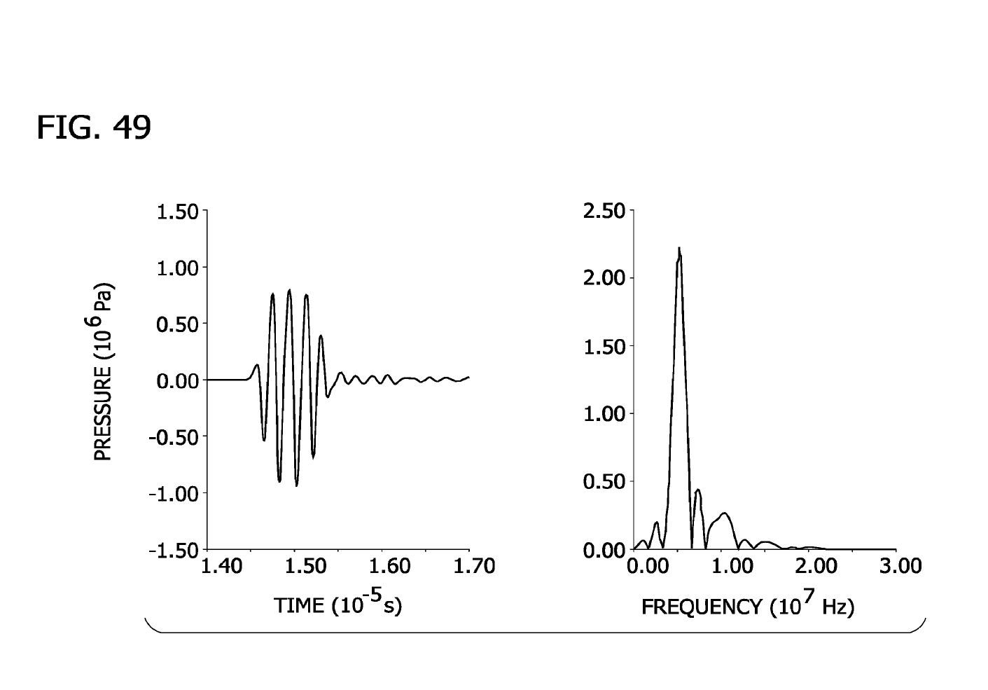

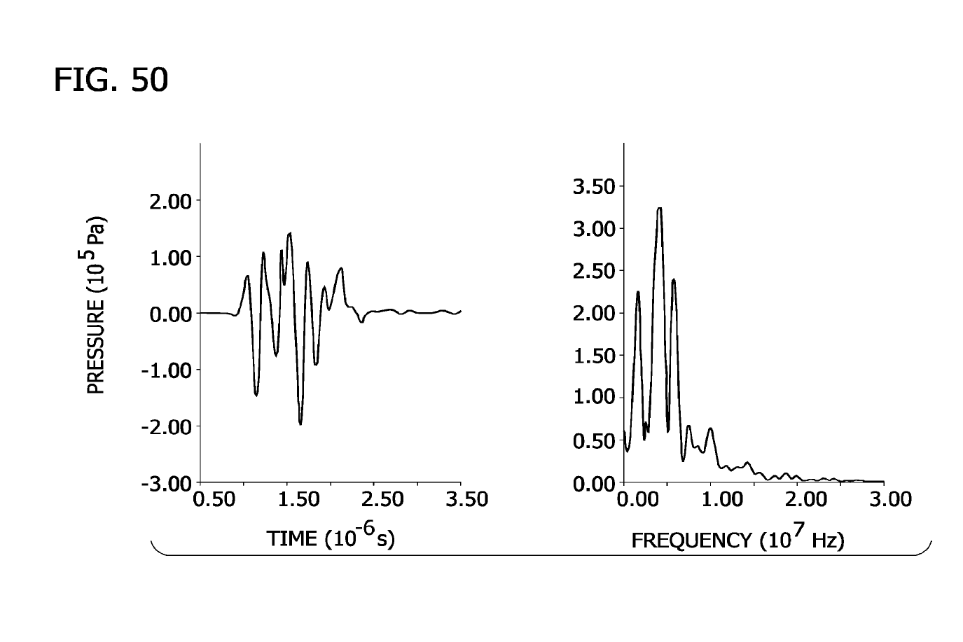

Here we introduce a driving method for Echo PIV harmonic imaging: triangular pulse driving. Rectangular pulses are commonly used as the input signals for ultrasound transducers because they can be easily implemented through hardware. A triangular pulse carries less power than rectangular pulse, which minimizes the possibility of bubble rupture and reduces ultrasound intensity especially at high frame rates. Also, a triangular pulse is very efficient in triggering strong backscatter from the contrast agents, especially the sub and higher harmonic contents. FIG. 48 shows a 3-cycle 5 MHz triangular pulse in both time and frequency domains. A linear array ultrasound transducer model was designed and its performance modeled to simulate the focused ultrasound beam propagating in water. FIG. 49 shows the simulated pressure wave form at the 2.2 cm deep focal point by using the triangular pulse as the input signal. Nonlinear acoustic emissions from microbubbles were studied based on a modified Rayleigh-Plesset (RP) equation, which describes the backscatter of a single bubble. The RP equation was extended to a formulation that allows inclusion of an encapsulating shell of arbitrary thickness, density and viscosity. FIG. 50 shows the calculated bubble backscatter by using the pressure at the focal point for excitation. Very strong subharmonic and ultraharmonic contents were generated, and the 2^{nd} harmonic was relatively weak. This shows that a backscatter method focusing on subharmonic and ultraharmonic components of the backscatter signal should be useful in detecting bubbles within blood, and especially near interfaces such as tissue interfaces.

Parallel Beams Scanning Method for Improving Echo PIV Frame Rate in a Large FOV

The commonly used method for improving ultrasound imaging frame rate is to reduce the imaging depth and the number of scan lines since the ultrasound velocity in tissue media limits the pulse repetition frequency, and a small number of scan lines require less time for backscatter data acquisition. This method works well for peripheral vascular imaging, since the location of vascular is relatively shallow and the bubble image in a small window may provide enough information for successful Echo PIV measurements. However, for cardiac imaging and deep vascular imaging, the field of view (FOV) needed is relatively large, and alternative methods must be proposed to increase the frame rate.

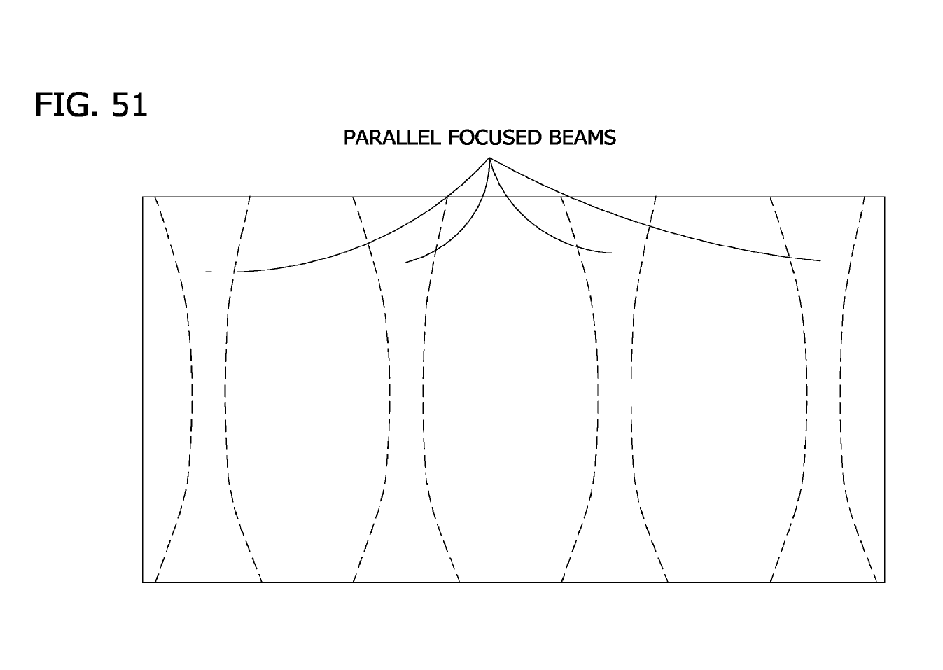

Here, we introduce the parallel beam scanning concept into Echo PIV to improve the frame rate. For conventional linear arrays, a certain number of transducer elements will be fired in sequence to form focused ultrasound beams to scan through the whole FOV In parallel beam scanning, several groups of transducer elements will be fired simultaneously, and several focused ultrasound beams will be generated at the same time to scan through a relatively small FOV, thus improving the frame rate by a factor of the number of simultaneous beams. FIG. 51 shows 4 parallel focused beams scanning a large FOV which might improve the frame rate by a factor of 4.

Echo PIV Techniques Using DICOM-Coded Ultrasound B-Mode Images

While, an echo particle image velocimetry (Echo PIV) technique has been established for non-invasive, in vivo multi-component flow visualization, it relies on the analysis of radio frequency (RF)-type data (pre-processed or raw data) from an ultrasound imaging systems. However, very few ultrasound imaging system manufacturers allow users to access RF data, thus limiting the usability the Echo PIV method. The vast majority of commercially available systems output ultrasound B-mode images use a standard called Digital Imaging and Communications in Medicine (DICOM), which has been defined for coding, handling, and storage of image data and communicating between clinical imaging systems. Under the DICOM standard, images from different origins are digitalized and created in a uniform image type that makes images from different manufacturers compatible. Further complicating the matter, a central feature of the DICOM standard is that various types of compression are applied to the ultrasound DICOM-compliant images, in order to diminish the volume of saved data. Through this automatic post-processing, important information is lost and noise is introduced into the image.

Such compression processes are usually regarded as lossy′ and lossless' for some of the algorithms like JPEG, run-length encoding, and lossless JPEG 2000; both however result in loss of image detail, which is assessed based the visual quality of the compressed images in clinical acceptability, diagnostic accuracy and human visual perception structural similarity. Note that the term lossless' here does not mean the compressed images are identical to the original image data in each pixel. Additionally, many platforms cannot provide multi-frame uncompressed or lossless compressed DICOM image outputs, either. While this is acceptable for clinical diagnostic use, it could lead to Echo PIV analysis failure. Recent studies have reported direct PIV analysis on post-processed B-mode images, but this has been limited to qualitative information such as left ventricular blood vorticity. Quantitative information is not available to determine the accuracy of the results. The lower quality of post-processed B-mode images, when compared to RF data, affects the performance of the Echo PIV algorithm affecting its accuracy.

Aside from compression issues, B-mode images obtained from RF raw data for the Echo PIV technique are different in content from DICOM images due to their inequable generation procedures. For example, specialized Echo PIV techniques such as the Wiener Filter are applied to RF data to detect the bubbles and enhance the signal-to-noise ratio (SNR) before reconstruction of B-mode images. To the contrary, DICOM images are subjected to techniques such as smoothing tools, gray level encoding algorithms, and so on to get best perceptive effectiveness and enhance clinical visualization. Such advanced post-processing techniques have caused loss of correlation information between image pairs of particles and may usually disable the Echo PIV analysis on them.

The PIV method consists of three primary steps: (1) Particle image acquisition; (2) Cross correlation; and (3) Particle displacement estimation. The velocities of particles are calculated by multiplying the particle displacements by image frame rate. Usually averaging operations, including the following, are applied to any of the steps above to improve the quality of the PIV results: (1) image averaging; (2) CC maps averaging; and (3) velocity averaging.

a. Flow Setup and Imaging Apparatus

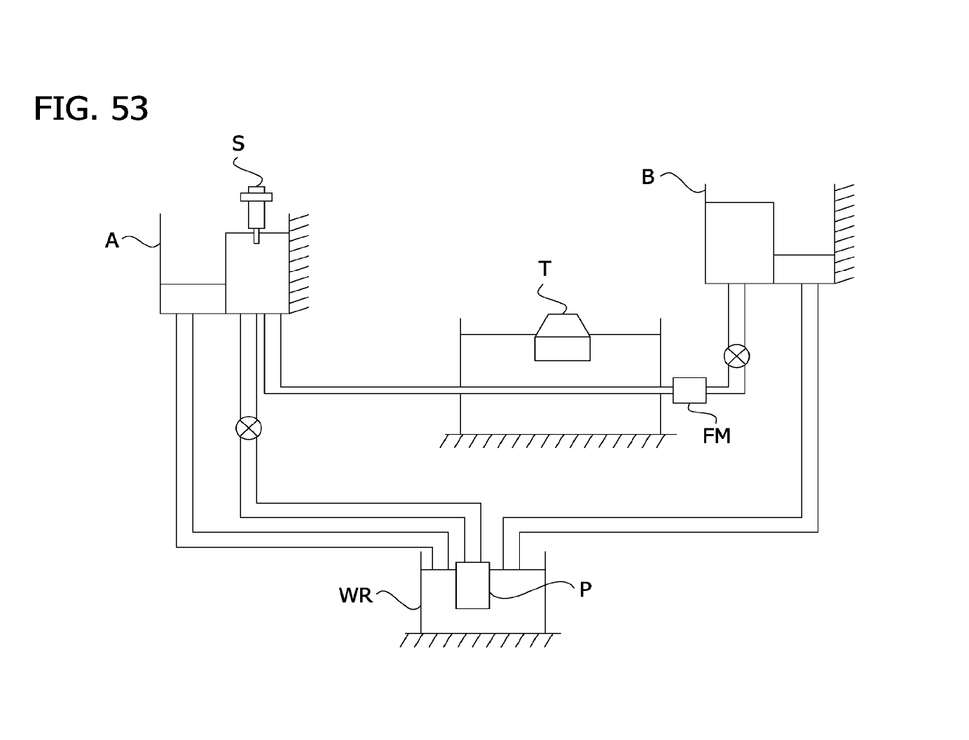

Fully developed laminar pipe flow in a long, straight, rigid, acrylic tube was first tested to validate the method. A sketch of the setup is shown in FIG. 53. In the setup, water was continuously circulated through the acrylic tube with a fixed head differential between two water tanks, generated by a mini-pump (Active AQUA Pump PU800, Hydrafarm Inc., Petaluma, Calif.) sitting in a water reservoir. The flow rate could be adjusted by a shunt valve and measured by a measuring cup and a timer. The acrylic tube was 1.8 m long with an inner diameter of D=9.5 mm; the tube entry length was set to 1.2 m to guarantee a fully developed laminar flow in the region of imaging. Bolus injections of approximately 0.1 ml of Optison® microbubble agents (GE Healthcare) were introduced as needed into water tank A and then well mixed by stirring the water before entering into the tube for imaging. Meanwhile, an US-friendly bifurcating silicon tube with patient-specific carotid artery junction geometry was also used for pulsatile flow studies, and the setup is shown in FIG. 53. A pulsatile blood pump (model 5F3305, Harvard Apparatus, Mass.) was used to supply the water flow. Optison® microbubbles (0.2 ml) were directly injected into the reservoir and well mixed by stirring the water for each test.

A SonixRP system (Ultrasonix Inc., Rich-mond, BC, Canada), using a 1-D, 128-element linear array transducer (L14-5/38), which is working at a center frequency of 10 MHz, was used to acquire both RF and lossy compressed DICOM data from the imaging of two flow models. The system is capable of both raw (RF) and DICOM image data outputs, thus allowing same image cycle record loops to be saved in both formats. The Echo PIV analysis results from these two data types were then compared to theory to validate the method for laminar flow results. Finally, the pulsatile flow model has also been studied to see the improvements these methods provide on the flow velocity vector mapping in more complex flow fields. For the studies of pulsatile flow, there is no simple theoretical calculation for the velocity distribution, but since the RF-based PIV results have been previously validated by Optical PIV measurements they are used herein as a reference to measure the performance of the new DICOM-based Echo PIV analysis algorithms.

b. Testing Environments on the Ultrasound Imaging Platforms

An important issue when analyzing DICOM-coded B-mode images using the Echo PIV method is reduction of the effects of noise introduced by the processing of images performed by the ultrasound systems. The post-processing of DICOM B-mode images is automatically conducted by ultrasound systems and results in a loss of information. Selection of the most appropriate values for the parameters for imaging may be helpful in reducing the noise introduced by such processing. For example, it has been found beneficial to set the ‘Clarify’ level to ‘OFF’ to deactivate the edge detection and smoothing post-processing filter. Choosing the frame rate as high as possible also achieves better velocity vectors.

c. Theoretical Velocity for Laminar Pipe Flow

The new methods are validated by matching their results to laminar flow theory in an interrogation plane parallel to the centerline plane of the tube. The theoretical expression of the flow velocity profile in such plane is given as follows at any point (−d, y) on the plane,

v(−d,y)=(1−(d²+y²)/a²)v₀ (Eqn. 7)

where d is the distance between the scanning and centerline planes, v₀ is the maximum velocity along the centerline of the tube, and a=D/2. The maximum and mean velocities of the imaging plane are given by

vₘ=v₀(1−d²/a²),vₐ=2v₀(1−d²/a²)/3 (Eqn. 8)

respectively. We note that the mean velocity vₐ is two-third of the maximum velocity vₘ in the imaging plane.

d. Modified Processing of Cross-Correlation Maps

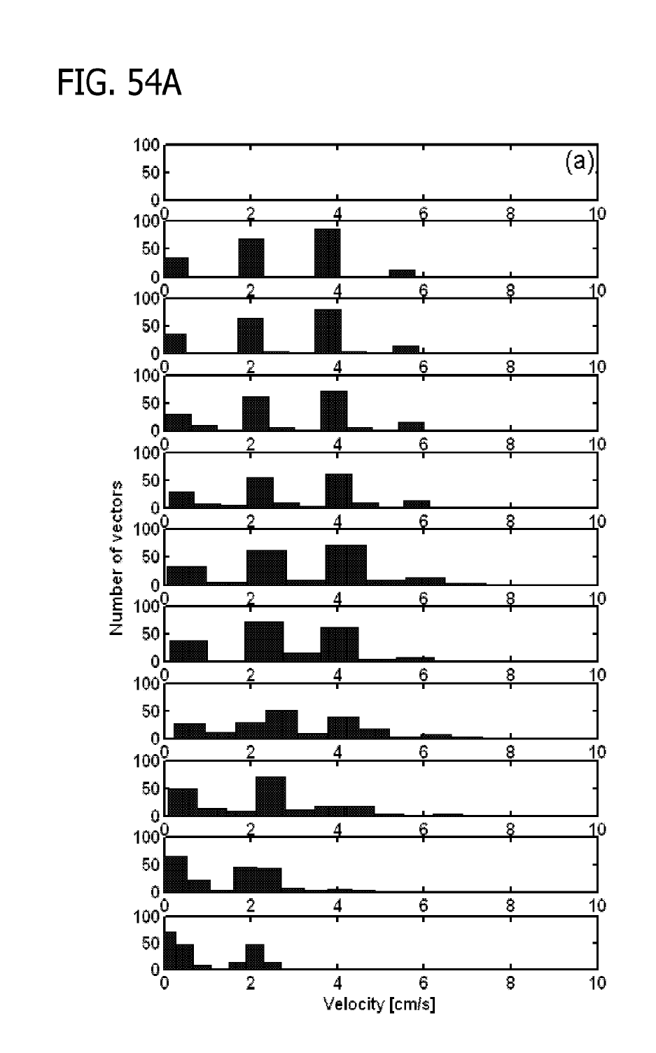

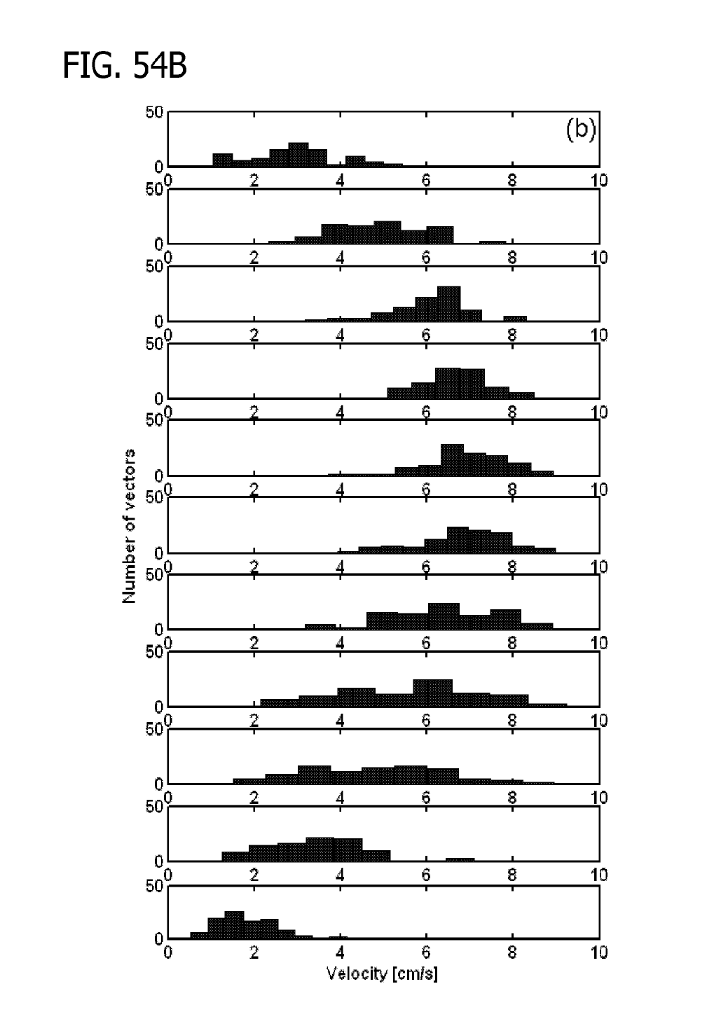

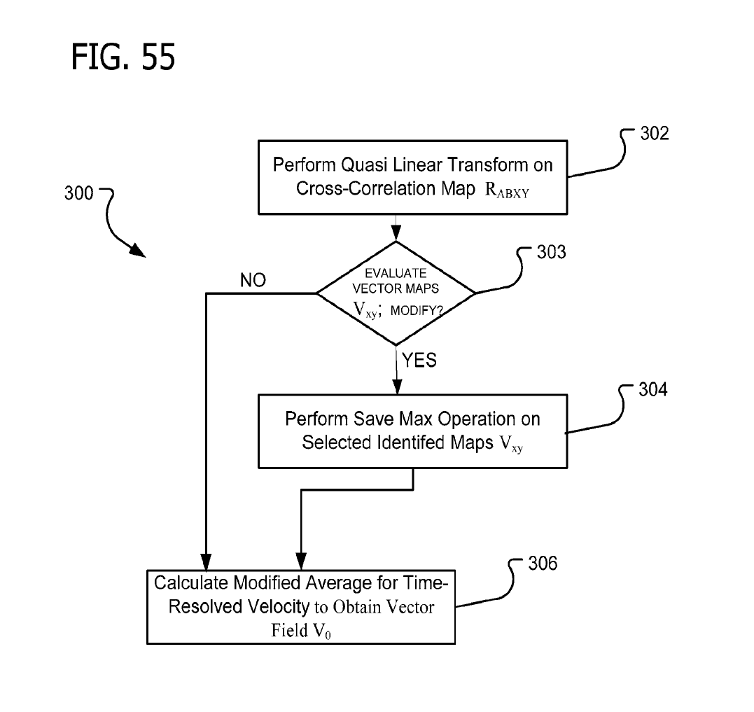

The cross-correlation (CC) map generated by the Echo PIV analysis of RF data has a high signal-to-noise ratio (SNR) as shown in FIG. 54A. In contrast, the CC map generated by applying the Echo PIV analysis to DICOM B-mode images has a low SNR as see in FIG. 54B. A low SNR makes the quantification of microbubbles displacement difficult affecting in turn the accuracy of the results. In order to overcome the low SNR in DICOM B-mode images, one embodiment of a methodology 300 as presented in FIG. 55 may be employed. First, a quasi-linear transformation is performed on each cross-correlation map R_{ABXY} (CCQLT Transformation) as indicated in operation 302. After transformation, the corresponding vector maps V_{xy} are evaluated at 303 and any vector maps having a missing vector or having a vector which is substantially different than an adjacent vector are identified for modification. Second, a “save maximum value” operation is performed on selected multiple image pairs as indicated in operation 304 for the identified vector maps having missing or different vectors which need to be modified. Third, a modified average method, which combines both average velocity and average correlation methods, is performed for the time-resolved velocity field as indicated in operation 306 to obtain the final mean velocity vector field V₀ of all the interrogated image pairs. Each of these steps is described in greater detail below.

1. New Echo PIV Methods: Quasi-Linear Transformation on Cross-Correlation Map (CCQLT Transformation)

The first DICOM-specific modification to the standard Echo PIV methodology involves the cross-correlation (CC) maps between image pairs. In one embodiment, the new method combines a high-pass filter and a quasi-linear transformation given by

$${R}^{′} ( i , j ) = \{ \begin{matrix}\frac{R ( i , j ) - k \cdot {R}_{m}}{( 1 - k ) {R}_{m}} , R ( i , j ) > k \cdot {R}_{m} \\ 0 , R ( i , j ) \leq k \cdot {R}_{m} ,\end{matrix} \qquad ( Eqn . 9 )$$

where R(i, j) and R′(i, j) are CC coefficients before and after transformation, Rₘ is the peak value of R(i, j) and k is a defined reduction ratio that can be chosen between 0 and 0.95. Under this transform “noise” components, defined as R(i, j)≦kRₘ, are set to zero (i.e. removed). This transformation is referred to as quasi-linear because it includes a linear component:

$$\frac{R ( i , j ) - k \cdot {R}_{m}}{( 1 - k ) {R}_{m}} ,$$

and a non-linear component: R(i, j)≦k·Rₘ. The retained peaks then mainly consist of the most significant CC peak(s), which are further enhanced by normalization with base (1−kRₘ). The high-pass filter retains the first several peaks in each CC map; and the normalization stretches such peaks to the amplitudes close to 1 as shown in FIG. 54C.

This modified Echo PIV processing technique has two advantages when analyzing DICOM images over the original Echo PIV method. First, it removes noise content from the CC map while preserving useful information. Second, it increases the amplitude of the main peak in the CC map, extends the first peak values with their neighbors in each map to a higher level, and, through an average method in the next step, each CC peak will be counted as a maximum for a displacement calculation. The extension of the weak CC peaks here also helps detect velocity vectors close to the wall, especially under the circumstances of high flow rates but low imaging frame rates.

2. New Echo PIV Methods: ‘Save Max’ (SM) Operation on Multiple Image Pairs

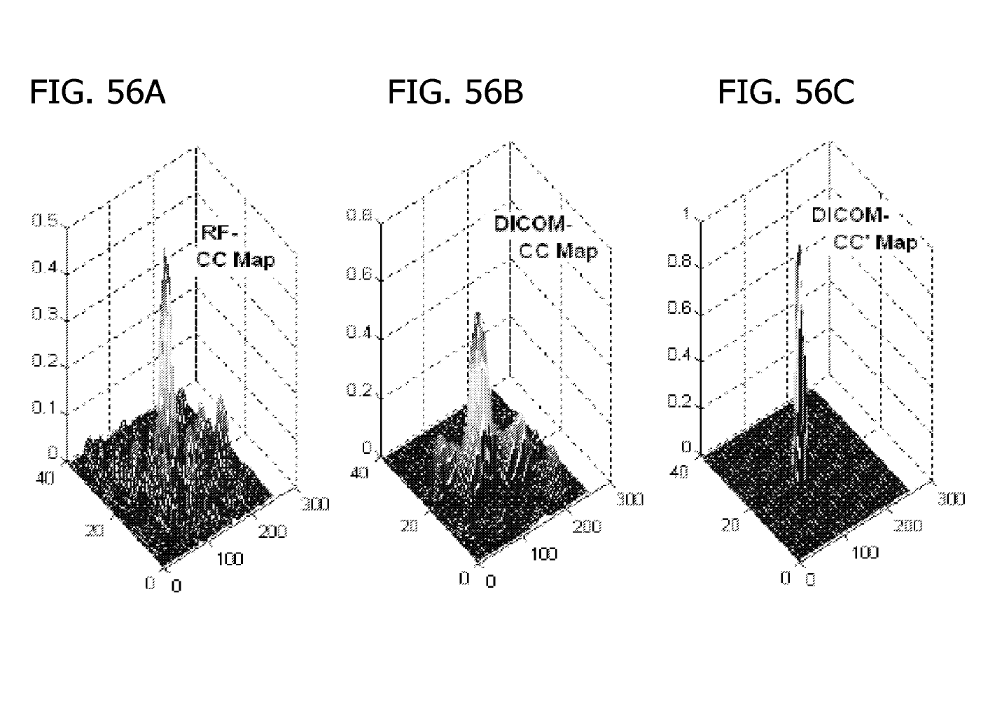

The Echo PIV analysis on single DICOM image pairs produces flow field maps with very sparse vectors even after CCQLT. No vector has been detected at many of the positions, whereas the RF-based method can typically provide reliable vectors throughout. Additionally, the detected vectors from DICOM could be either erroneous or valid. FIG. 56B shows histograms of the velocity vector distributions computed from 200 single DICOM image pairs at discrete increments across the diameter of the laminar-flow tube model, before applying filters. FIG. 56C illustrates histograms of velocity vector distributions computed from 200 single DICOM image pairs at the same radia locations of FIG. 56B but after filtering. Clearly FIG. 56B lacks the expected parabolic profile associated with laminar flow. This profile is clearly seen in FIG. 56A, showing the vector histograms at the same radial locations but after filtering from RF-based PIV estimation, which has been demonstrated accurate in previous work and is used here as a control standard. Similar features are seen in the vector distributions obtained for DICOM image analysis from other flow types, which suggests that the maximum detected velocities from DICOM data are very close to those with largest detection probability from RF data. Thus, a ‘save max (SM)’ operation is performed on the vector maps between the PIV results from single image pairs as follows:

vₙ₊₁(m,l)←max{vₙ(m,l),vₙ₊₁(m,l)} (Eqn. 10)

where vₙ(m,l) is calculated velocity at (m,l) for nth image pair.

3. New Echo PIV Methods: Modified Average Method for Time-Resolved Velocity Field

The Echo PIV technique we developed for analyzing RF data is based on the averaging of velocity information. This method works well when analyzing RF data however the lower quality of DICOM images affects the performance of the algorithm. Only a few instantaneous vectors can be visualized from single DICOM image pairs. Thus, a direct average operation on such vector maps will lead to prediction of a mean field with velocity magnitudes far below their actual values. Additionally, the average correlation method combined with the SM operation appears to produce vector maps that are less smooth than expected. A modified average method, which combines both average velocity and average correlation methods, is used to improve the time-averaged flow field.

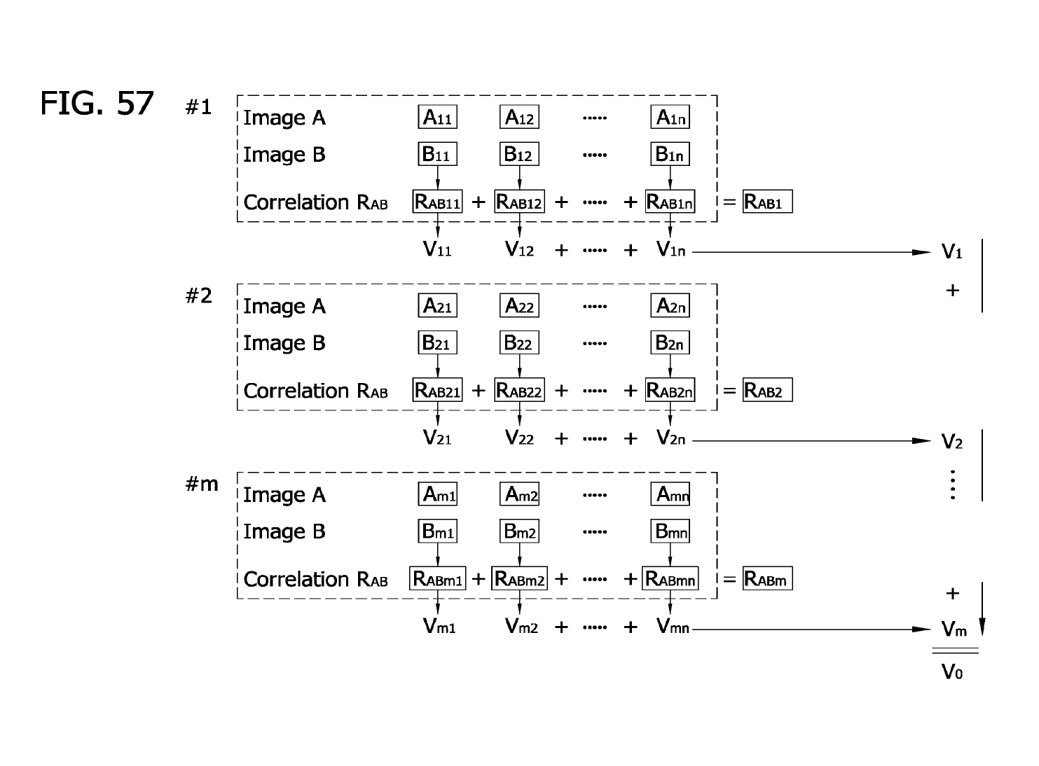

A schematic diagram graphically outlining this method is shown in FIG. 57. Sequential image pairs (A₁₁, B₁₁; in general, A_{xy}, B_{xy}) are separated into m groups, each of which has n pairs. Within these m groups, the average correlation method is applied to sequential image pairs (A_{xy}, B_{xy}) to generate a correlation map R_{ABXY} for each image pair (A_{xy}, B_{xy}). Each correlation map R_{ABXY} corresponds to a vector map V₁₁, V_{xy}, to which the ‘Save Max’ operation is applied. The maximized vector maps V_{xy} are collected into m temporary spatial vector maps V₁, V₂, . . . . Vₘ. These temporary vector maps are then averaged directly by the standard velocity average method to obtain the final mean velocity vector field V₀ of all the interrogated image pairs.

For the laminar flow model, one hundred image pairs (101 successive frames A, B) are adopted for ensemble averaging using the new algorithms with different group sizes for DICOM images, and using the direct velocity average method (original algorithms) for RF-reconstructed images for comparison. For the instantaneous velocity field of the pulsatile flow, only the correlation average method is applied to the every two successive image pairs and results from both RF and DICOM data are compared.

Thus, in one form, the invention comprises a method for processing Digital Imaging and Communications in Medicine (DICOM) encoded ultrasound B-mode images representing a fluid flow of a plurality of particles. A plurality of DICOM encoded ultrasound B-mode sequential images representing sequential image pairs (A_{xy}, B_{xy}) of a plurality of particles is received, such as by a computer 19A. The plurality of DICOM sequential image pairs are grouped into a plurality of M groups of images, wherein each M group comprises a plurality of N sequential image pairs. Within each group, the sequential image pairs (A_{xy}, B_{xy}) are correlated to create N cross correlation maps (R_{ABXY}). Within each group, an average cross-correlation transformation is applied to each cross correlation map (R_{ABXY}) to create an image pair vector map (V_{xy}) for each image pair. A maximizing operation is applied to one or more of the N adjacent image pair vector maps (V_{a,b}, V_{a+1,b+1}) to create a modified image pair vector map (V′_{xy}) for the one or more of the N image pairs. For each group, the image pair vector maps and the modified image pair vector maps are combined to create a corresponding temporary vector map (Vₘ). The temporary vector maps are averaged to obtain a mean velocity vector field (V₀) of the sequential image pairs representing a fluid flow of a plurality of particles.

Thus, in one form, the invention comprises an apparatus for processing Digital Imaging and Communications in Medicine (DICOM) encoded ultrasound B-mode images representing a fluid flow of a plurality of particles. A tangible computer readable storage medium stores DICOM encoded ultrasound B-mode images, said medium storing processor executable instructions comprising:
instructions for receiving a plurality of DICOM encoded ultrasound B-mode sequential images representing sequential image pairs (A_{xy}, B_{xy}) of a plurality of particles;
instructions for grouping the plurality of DICOM sequential image pairs (A_{xy}, B_{xy}) into a plurality of M groups of images, wherein each M group comprises a plurality of N sequential image pairs;
instructions for correlating, within each group, the sequential image pairs (A_{xy}, B_{xy}) to create N cross correlation maps (R_{ABXY});
instructions for applying, within each group, an average cross-correlation transformation to each cross correlation map (R_{ABXY}) to create an image pair vector map (V_{xy}) for each image pair (A_{xy}, B_{xy});
instructions for performing a maximizing operation to at least one or more of the N adjacent image pair vector maps (V_{xy}) to create a modified image pair vector map (V′_{xy}) for each N image pair (A_{xy}, B_{xy});
instructions for combining, for each group, the image pair vector maps (V_{xy}) and the at least one or more modified image pair vector maps (V′_{xy}) to create a corresponding temporary vector map (Vₘ) for each group; and
instructions for averaging the temporary vector maps (Vₘ) to obtain a mean velocity vector field (V₀) of the sequential image pairs (A_{xy}, B_{xy}) representing a fluid flow of a plurality of particles; and

a processor 19A for accessing the DICOM encoded ultrasound B-mode images stored on the tangible computer readable storage medium and for executing the executable instructions stored on the tangible computer readable storage medium to process the accessed DICOM images.

Validation for Fully Developed Laminar Pipe Flow Model

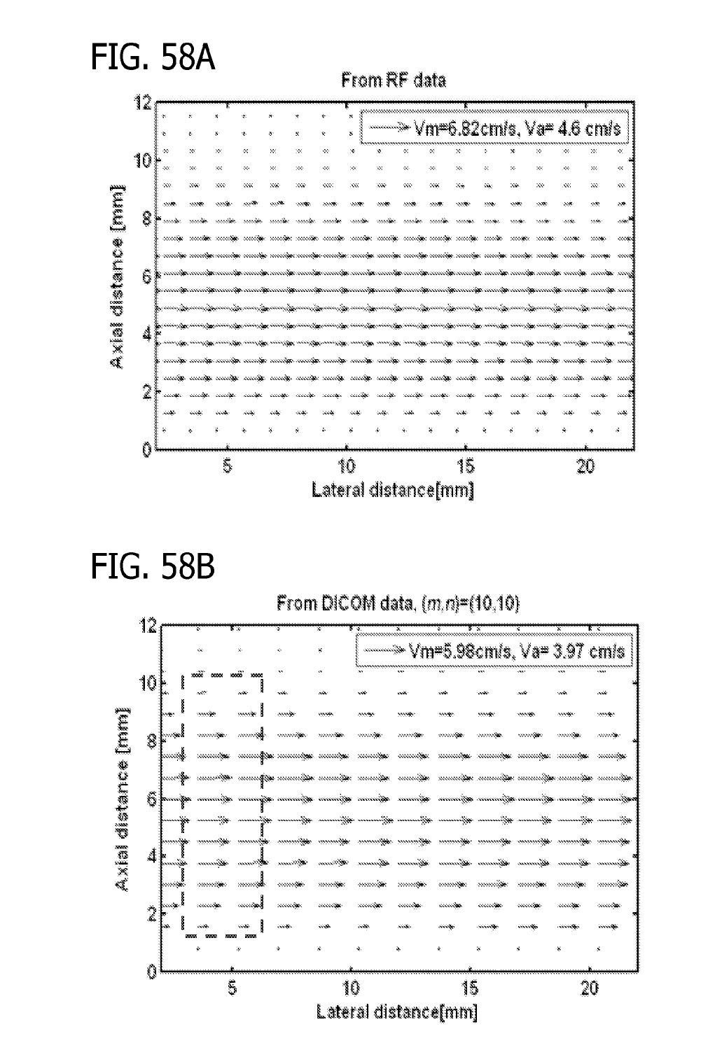

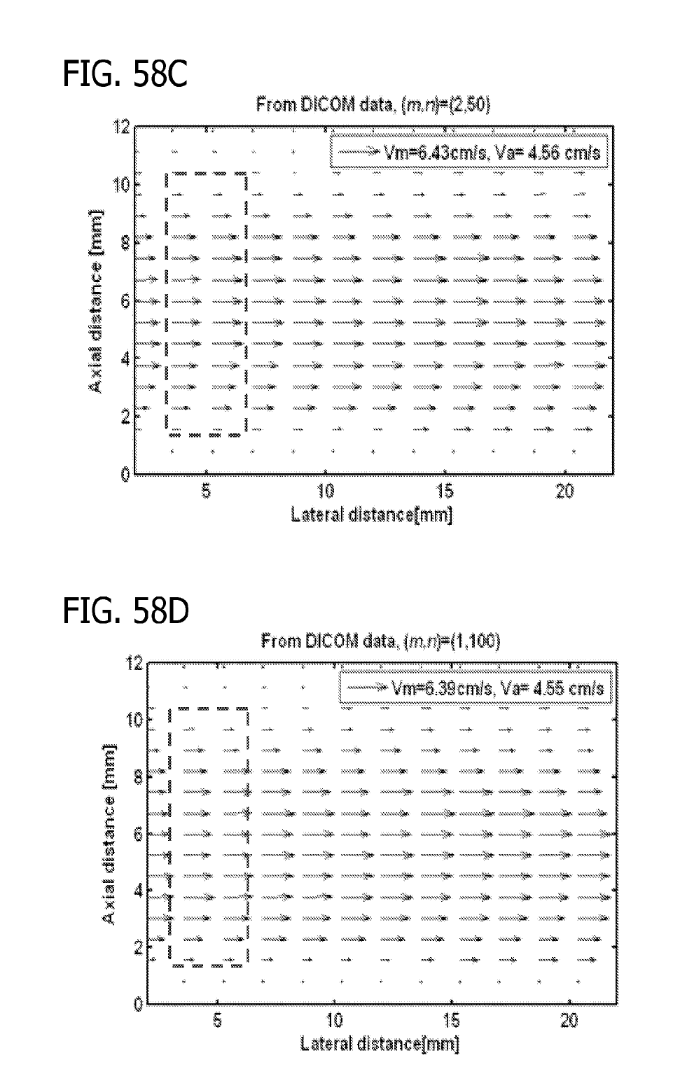

The echo PIV vector maps for fully developed laminar flow model are shown in FIGS. 58A-D. The map from RF data is provided in FIG. 58A as a reference. Maps from DICOM images are calculated with different group sizes n=10, 50 and 100 pairs per group, and are exhibited in FIGS. 58B, 58C, and 58D, respectively. Some imaging parameters are listed as follows: image sector size 60% (image width=23.04 mm), gain 60%, dynamic range 70%, beamline density 0.5, focal depth 3.0 cm, and image frame rate 183 FPS. A reduction ratio k of 0.3 was selected for best performance. The flow rate is adjusted to 200 ml/min, for which the predicted maximum and mean velocities are 6.62 cm/s and 4.41 cm/s for the imaging plane with inner tube diameter 8.7 mm. The measured maximum velocities are 6.82 cm/s (overestimated by +3.02%) from RF data; and 5.98 cm/s (−9.67%), 6.43 cm/s (−2.87%) and 6.39 cm/s (−3.47%) for DICOM data; and the mean velocities are 4.6 cm/s (+4.31%), 3.97 cm/s (−9.98%), 4.56 cm/s (+3.40%) and 4.55 cm/s (+3.17%) for the four maps, respectively. It is demonstrated that the maps in FIGS. 58A, 58C, and 58D are measurements with errors less than ±5%. However, for the image pairs separated into 10 groups with 10 pairs each, the velocities are underestimated with errors near 10%. Notably, the velocity vectors look more smooth for n=10 and 50 than that for n=100, especially the areas enclosed by red dashed lines. This suggests a tradeoff exists, in that large groups with more image pairs have less missing velocity vectors and thus guarantee more accurate measurements, but more image groups will make the vector smoother.

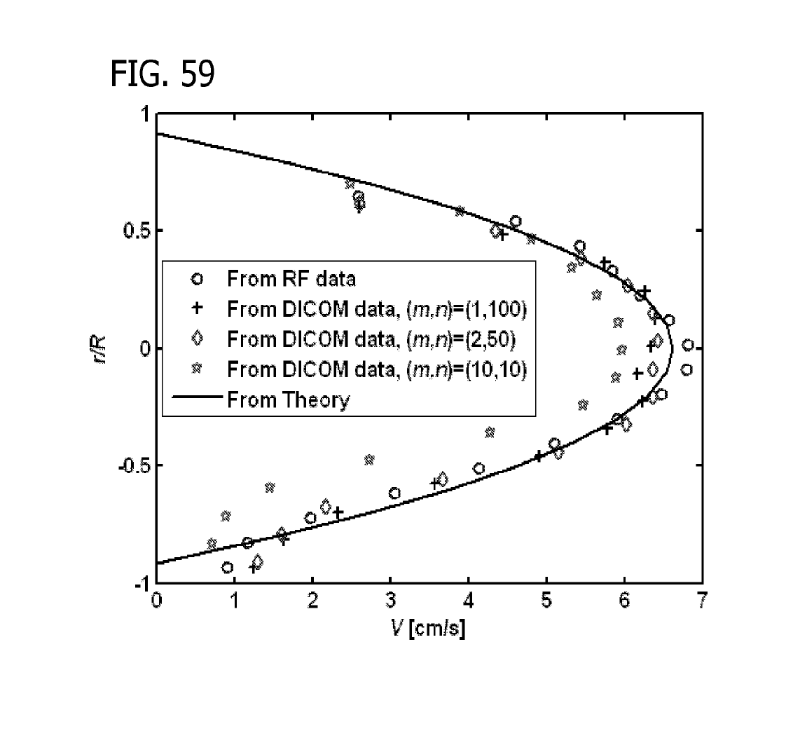

The results are further compared in flow velocity profiles, as shown in FIG. 59. The DICOM data leads to a good estimation under the Echo PIV analysis with the new algorithms and large groups of n=50 and 100 but leads to underestimation using smaller image groups n=10. Except for the missing information in the near wall region, the measurements agree well with the profile theory with acceptable errors.

Improvement of the Vector Map for Pulsatile Flow Model

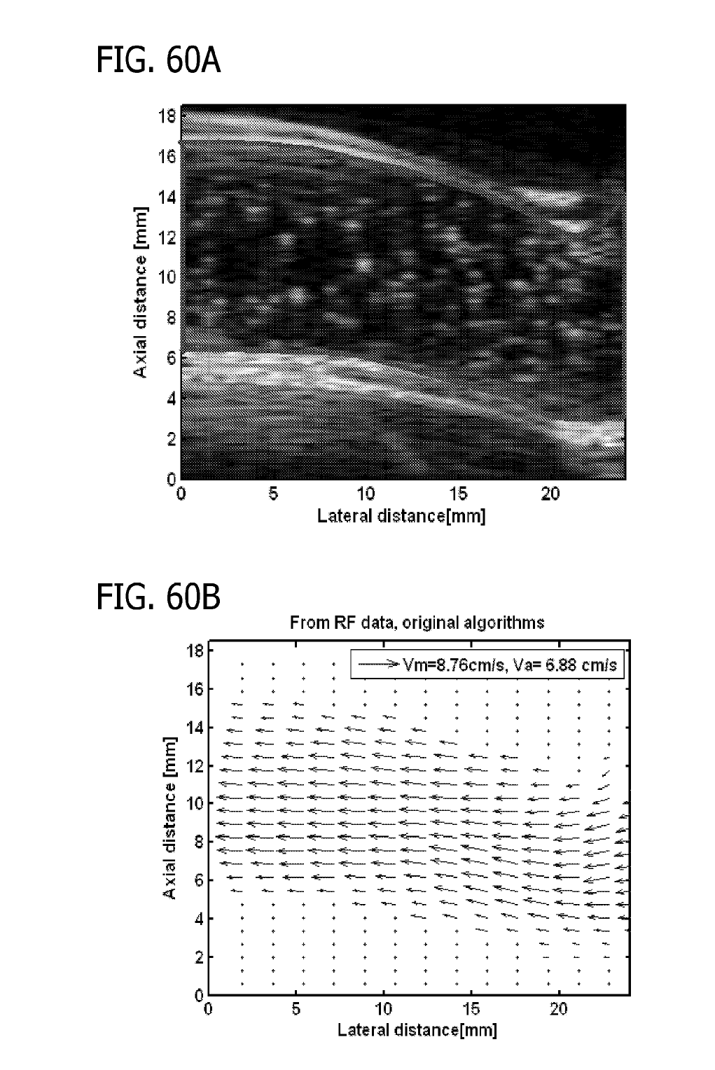

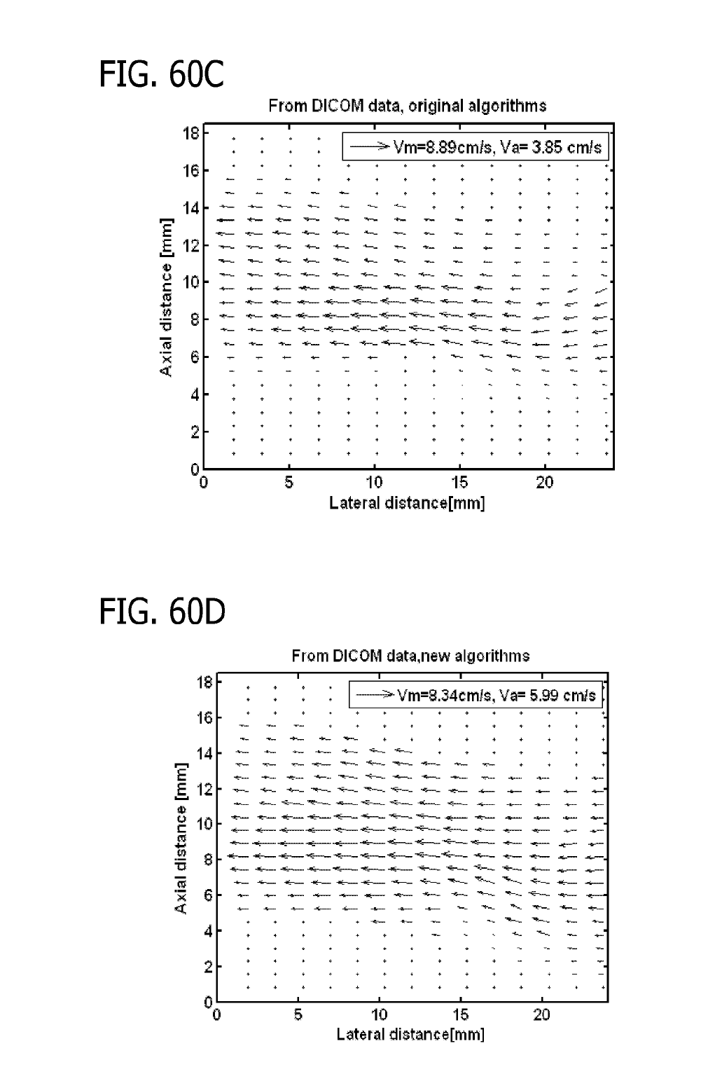

The pulsatile flow settings are given as follows: pump stroke 15, rate 20, diastole 45/55, and flow rates 0.18˜0.32 L/min. Both the stroke and rate of the tested flows are much lower than physiological conditions and, as a result, forward and backward flows may be visually and clearly seen on the US machine screen from particle movements prior to any analysis. The interrogation area for Echo PIV analysis is shown the region enclosed by white lines in the B-mode image of FIG. 60A. FIG. 60B indicates that original Echo PIV algorithms on RF data have very good performance on vector mapping, while the same algorithms collects approximately less than half vectors of smaller values for DICOM data, as exhibited in FIG. 60C. While still not as accurate as the results from RF data in some vectors, the vector map in FIG. 60D has been greatly improved by regaining almost all the missing velocity vectors as compared to FIG. 60B using the new Echo PIV algorithms.

Observations Regarding Echo PIV Analysis on DICOM B-Mode Images

Echo PIV analysis on DICOM-coded US images is improved by using the new algorithm developed herein. This work has been validated against theory for fully-developed laminar pipe flow. The feasibility study also makes Echo PIV technique potentially applicable in numerous US imaging platforms where RF image data are not available. The availability of an Echo PIV algorithm for the analysis of DICOM B-mode images may have a tremendous impact for it allows the method to be applied to ultrasound images independent of the manufacturer, making it accessible to any researcher and clinician. The modified Echo PIV algorithm can accurately generate hemodynamic vascular profiling information through the analysis of DICOM ultrasound B-mode images.

### APPENDIX A

Design of the Wiener Filter for Improving B-Mode Microbubble Images

A typical B-mode microbubble image is shown in FIGS. 5A and 9A. The bubble images are reconstructed from RF signals, which are a series of echo pulse from many microbubbles. Unfortunately, those echo pulses are corrupted by the noise effect and the interferences between neighboring bubbles.

The goal of our custom-designed wiener filter is to filter out the noise that has corrupted a signal. Typically the wiener filter is designed in frequency domain, where it has the traditional form,

$$G ( w ) = \frac{{H}^{*} ( w ) {P}_{s} ( w )}{{\vert H ( w ) \vert}^{2} {P}_{s} ( w ) + {P}_{n} ( w )} \qquad ( Eqn . A1 )$$

where, H(w) is the Fourier transform of the point-spread function (PSF), Pₛ(w) and Pₙ(w) are the power spectrums of the signal and the noise process, respectively, obtained by taking the Fourier transform of the signal and noise autocorrelation.

Therefore, it is generally assumed that the spectral properties of the original signal and the noise are known. However, in the obtained RF echo signals, it is difficult to exactly estimate the spectral properties of signal and noise. So we design the wiener filter in such a form,

$$G ( w ) = \frac{{H}^{*} ( w )}{{\vert H ( w ) \vert}^{2} + σ} \qquad ( Eqn . A2 )$$

where σ is signal-noise factor, not dependant on the frequency.

Although the wiener filtering is generally not used in the reconstruction of B-mode images with many reflection objects, the advantage in echo PIV images is that the microbubble image comprises many bubbles with similar properties, that is, the echoed pulses from the bubbles are similar.

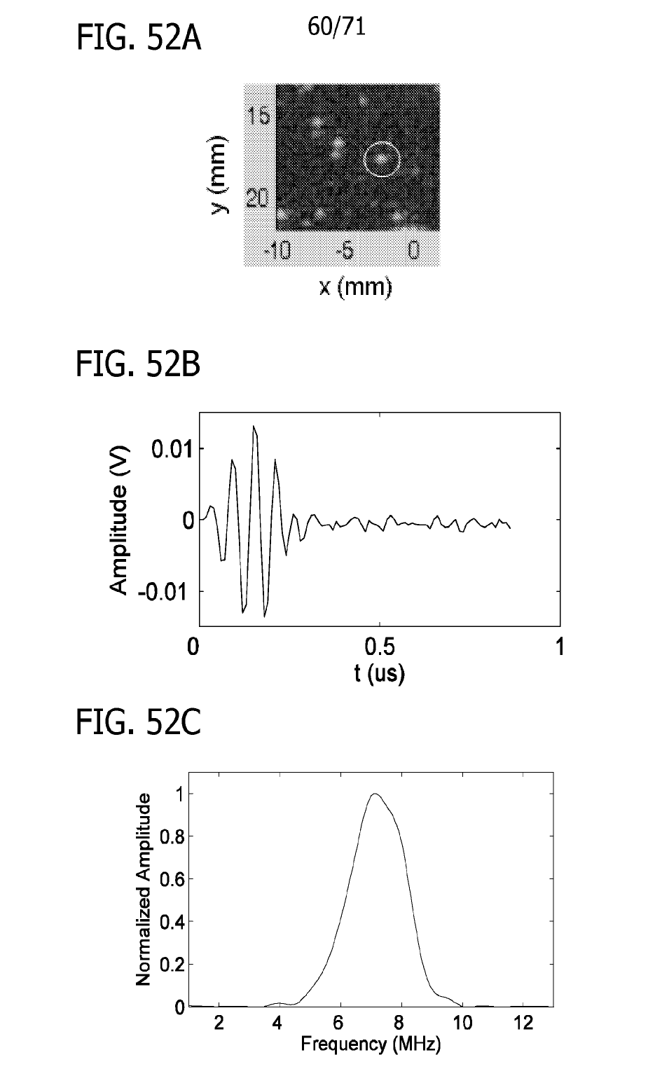

We approximate the PSF by estimating the echoed pulse from a steady fluid with very low concentration of microbubbles, in the uttermost extent to reduce the noise effect and the interferences between bubbles. The echo pulse and its spectrum are shown in FIG. 52. FIG. 52 illustrates an estimation of PSF from B-mode microbubble image: 52A shows microbubble images; 52B shows echo pulse from circled bubble in 52A; and 52C shows spectrum of echo pulse in 52B, according to embodiments of the invention.

In this way, we obtain the approximated wiener filter from Eq. A2, which is dependant on signal-noise factor σ (typically between 1˜100 for the echo PIV images).

### APPENDIX B

Design of Filters for Improving Vector Field

Global Filter

For the real flow field, generally it can be assumed that the velocity difference between the neighboring velocity vectors is smaller than a certain threshold εₜₕᵣₑₛₕ,

|U(i,j)−U_{avg}|>εₜₕᵣₑₛₕ (Eqn. B1)

where U(i,j) is the velocity at each position (i,j), U_{avg} is the average value of total vectors in the flow field, and εₜₕᵣₑₛₕ=C_{g}U_{std}, in which, C_{g} is a constant (assigned by the user) and U_{std} is the standard deviation of vector values within the flow field.

The vector is identified as an outlier if its value U(i,j) meets the requirement in Eq. B1. Therefore, the global filter applies physical limitations to remove all the impossible data, located beyond the range [U_{avg}−C·U_{std}, U_{avg}+C·U_{std}]. The constant C_{g} depends on the velocity distribution and the quality of cross-correlation, with the recommended values listing in Table B1.

TABLE B1
Threshold values for different types of filters

| Filter Type | Threshold | General Value Range* |
|---|---|---|
| Global Filter | C_{g} | 2~3 |
| Local Filter | Cₗ | 1.5~3 |
| SNF | ε_{SNF} | 1.1~1.3 |

Note*:
For global and local filters, the smaller the threshold value, the more vectors will be identified as outliers; for, SNF, the greater the value, the more vectors will be detected.

In addition to assigning the constant C value, the upper and lower limit could also be determined manually by choosing four points (limits for u and v component of velocity) on the u-v velocity map. This method works better when some flow information is known a-priori, for example when u or/and v component has only positive or negative values in some regions.

Local Filter

The local filter aims to detect those erroneous vectors that could not be detected by the global filter (i.e., their values are in the possible range), but are surrounded by some correct vectors. In this way, these vectors could be detected by comparing their values with the surrounding values. Typically, a 3×3 pixel neighborhood with eight nearest vectors or 5×5 with 24 neighbors is selected. The velocity vector is deemed erroneous and rejected if the following condition is satisfied:

|U(i,j)−Uₙ|>ε_{n,thresh} (Eqn. B2)

where n represents the n^{th} unit in the vector field, U(i,j) is the value of detected vector at position (i,j) in the unit, Uₙ is the mean or median value of the surrounding vectors of vector (i,j), and εn,thresh is the threshold for the n^{th} unit, defined as ε_{n,thresh}=C₁·U_{n,std}, in which, C₁ is a constant selected by the user (see Table B1 for recommended value range) and U_{n,std} is the standard derivation of the neighbor vectors. Typically, depending on the definition Uₙ, the local filter has two types: local mean filter and local median filter.

Signal to Noise Filter

The global and local filter, in most cases, cannot detect all of the erroneous vectors in the vector field, for example, when there is a region with some outliers being together. For this reason, a signal to noise filter (SNF) is designed to especially detect those groups of erroneous vectors from bad cross-correlation due to the noise or particle mismatching. The vector will be re-classified as an outlier if the following criterion is satisfied:

Cₚₑₐₖ/C′ₚₑₐₖ<ε_{SNF} (Eqn. B3)

where Cₚₑₐₖ is the peak value in the cross-correlation map, from which a displacement of particle movement is determined, and C′ₚₑₐₖ is the peak value of the regions excluding the highest peak region (a small region near Cₚₑₐₖ, typically a 4×4˜6×6 pixels depending on the interrogation window size), ε_{SNF} is the threshold with a general value range listed in Table B1.

Actually, considering the fact that the cross-correlation quality is related to both the peak value of cross-correlation map and the shape of the peak profile, we could also define the SNF as,

Cₚₑₐₖ/(C′ₚₑₐₖ·Rₐᵣₑₐ)<ε_{SNF} (Eqn. B4)

where Cₚₑₐₖ and C′ₚₑₐₖ are the same with those in Eq. (B3), and Rₐᵣₑₐ is the ratio between the pixel areas with cross-correlation values greater than the half of the peak value and the whole pixel areas in cross-correlation map.

The above specification, examples and data provide a complete description of the structure and use of exemplary embodiments of the invention. Although various embodiments of the invention have been described above with a certain degree of particularity, or with reference to one or more individual embodiments, those skilled in the art could make numerous alterations to the disclosed embodiments without departing from the spirit or scope of this invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A method for processing Digital Imaging and Communications in Medicine (DICOM) encoded ultrasound B-mode images representing a fluid flow of a plurality of particles, comprising:
receiving a plurality of DICOM encoded ultrasound B-mode sequential images representing sequential image pairs (A_{xy}, B_{xy}) of a plurality of particles;
grouping the plurality of DICOM sequential image pairs (A_{xy}, B_{xy}) into a plurality of M groups of images, wherein each M group comprises a plurality of N sequential image pairs;
within each group, correlating the sequential image pairs (A_{xy}, B_{xy}) to create N cross correlation maps (R_{ABXY});
within each group, applying an average cross-correlation transformation to each cross correlation map (R_{ABXY}) to create an image pair vector map (V_{xy}) for each image pair (A_{xy}, B_{xy});
performing a maximizing operation to at least one or more of the N adjacent image pair vector maps (V_{xy}) to create a modified image pair vector map (V′_{xy}) for each N image pair (A_{xy}, B_{xy});
for each group, combining the image pair vector maps (V_{xy}) and the at least one or more modified image pair vector maps (V′_{xy}) to create a corresponding temporary vector map (Vₘ) for each group; and
averaging the temporary vector maps (Vₘ) of the groups to obtain a mean velocity vector field (V₀) of the sequential image pairs (A_{xy}, B_{xy}) representing a fluid flow of a plurality of particles.

2. The method of claim 1, wherein applying the average cross-correlation transformation comprises utilizing a transformation comprising:
$${R}^{′} ( i , j ) = \{ \begin{matrix}\frac{R ( i , j ) - k \cdot {R}_{m}}{( 1 - k ) {R}_{m}} , R ( i , j ) > k \cdot {R}_{m} \\ 0 , R ( i , j ) \leq k \cdot {R}_{m} ,\end{matrix}$$
where R(i, j) is a cross-correlation coefficient before the transformation, R′(i, j) is a cross-correlation coefficient after the transformation, Rₘ is a peak value of R(i, j), and k is a defined reduction ratio having a value between about 0 and about 0.95.

3. The method of claim 2, wherein utilizing the transformation comprises applying a removing operation such that noise components defined as R(i, j)≦kRₘ are set to zero.

4. The method of claim 1, wherein applying the average cross-correlation transformation comprises applying a high-pass filter and a linear transformation.

5. The method of claim 1, wherein applying the average cross-correlation transformation further comprises removing noise components.

6. The method of claim 1, wherein applying the average cross-correlation transformation comprises applying a transformation having a linear component and having a non-linear component, whereby a signal-to-noise ratio (SNR) of the DICOM encoded ultrasound B-mode images is increased.

7. The method of claim 1, wherein the received DICOM images comprise DICOM images without edge detection and without smoothing post-processing filtering.

8. The method of claim 1, wherein the maximizing operation comprises determining a maximum velocity between adjacent image pair vector maps as follows:
vₙ₊₁(m,l)←max{vₙ(m,l),vₙ₊₁(m,l)},
where vₙ(m,l) is calculated velocity at (m,l) for nth image pair.

9. An apparatus for processing Digital Imaging and Communications in Medicine (DICOM) encoded ultrasound B-mode images representing a fluid flow of a plurality of particles, said apparatus comprising:
a tangible computer readable storage medium for storing DICOM encoded ultrasound B-mode images, said medium storing processor executable instructions comprising:
instructions for receiving a plurality of DICOM encoded ultrasound B-mode sequential images representing sequential image pairs (A_{xy}, B_{xy}) of a plurality of particles;
instructions for grouping the plurality of DICOM sequential image pairs (A_{xy}, B_{xy}) into a plurality of M groups of images, wherein each M group comprises a plurality of N sequential image pairs;
instructions for correlating, within each group, the sequential image pairs (A_{xy}, B_{xy}) to create N cross correlation maps (R_{ABXY});
instructions for applying, within each group, an average cross-correlation transformation to each cross correlation map (R_{ABXY}) to create an image pair vector map (V_{xy}) for each image pair (A_{xy}, B_{xy});
instructions for performing a maximizing operation to at least one or more of the N adjacent image pair vector maps (V_{xy}) to create a modified image pair vector map (V′_{xy}) for each N image pair (A_{xy}, B_{xy});
instructions for combining, for each group, the image pair vector maps (V_{xy}) and the at least one or more modified image pair vector maps (V′_{xy}) to create a corresponding temporary vector map (Vₘ) for each group; and
instructions for averaging the temporary vector maps (Vₘ) to obtain a mean velocity vector field (V₀) of the sequential image pairs (A_{xy}, B_{xy}) representing a fluid flow of a plurality of particles; and
a processor for accessing the DICOM encoded ultrasound B-mode images stored on the tangible computer readable storage medium and for executing the executable instructions stored on the tangible computer readable storage medium to process the accessed DICOM images.

10. The Apparatus of claim 9, wherein the instructions for applying the average cross-correlation transformation comprises instructions for utilizing a transformation comprising:
$${R}^{′} ( i , j ) = \{ \begin{matrix}\frac{R ( i , j ) - k \cdot {R}_{m}}{( 1 - k ) {R}_{m}} , R ( i , j ) > k \cdot {R}_{m} \\ 0 , R ( i , j ) \leq k \cdot {R}_{m} ,\end{matrix}$$
where R(i, j) is a cross-correlation coefficient before the transformation, R′(i, j) is a cross-correlation coefficient after the transformation, Rₘ is a peak value of R(i, j), and k is a defined reduction ratio having a value between about 0 and about 0.95.

11. The Apparatus of claim 10, wherein instructions for utilizing the transformation comprise instructions for applying a removing operation such that noise components defined as R(i, j)≦kRₘ are set to zero.

12. The Apparatus of claim 9, wherein instructions for applying the average cross-correlation transformation comprise instructions for applying a high-pass filter and a linear transformation.

13. The Apparatus of claim 9, wherein instructions for applying the average cross-correlation transformation further comprise instructions for removing noise components.

14. The Apparatus of claim 9, wherein instructions for applying the average cross-correlation transformation comprise instructions for applying a transformation having a linear component and having a non-linear component, whereby a signal-to-noise ratio (SNR) of the DICOM encoded ultrasound B-mode images is increased.

15. The Apparatus of claim 9, wherein the stored DICOM images comprise DICOM images without edge detection and without smoothing post-processing filtering.

16. The Apparatus of claim 9, wherein instructions for performing the maximizing operation comprise instructions for determining a maximum velocity between adjacent image pair vector maps as follows:
vₙ₊₁(m,l)←max{vₙ(m,l),vₙ₊₁(m,l)},
where vₙ(m,l) is calculated velocity at (m,l) for nth image pair.
